(19) United States
(12) Patent Application Publication
(10) Pub. No.: US 2025/0263523 A1
(43) Pub. Date: Aug. 21, 2025

(54) NOVEL PLASTICIZERS, PROCESS FOR THE PREPARATION THEREOF, AND USE THEREOF

(71) Applicants: ORPIA INNOVATION, PARIS (FR); CENTRE NATIONAL DE LA RECHERCHE SCIENTIFIQUE, PARIS (FR); UNIVERSITE DE MONTPELLIER, Montpellier (FR); ECOLE NATIONALE SUPERIEURE DE CHIMIE DE MONTPELLIER, MONTPELLIER CEDEX 5 (FR)

(72) Inventors: Sylvain CAILLOL, MONTPELLIER (FR); Lucas JEGO, MONTPELLIER (FR); Vincent LAPINTE, CASTRIE (FR); Adélaïde GARTILI, MONTPELLIER (FR); Benoît BRIOU, MONTPELLIER (FR)

(73) Assignees: ORPIA INNOVATION, PARIS (FR); CENTRE NATIONAL DE LA RECHERCHE SCIENTIFIQUE, PARIS (FR); UNIVERSITE DE MONTPELLIER, Montpellier (FR); ECOLE NATIONALE SUPERIEURE DE CHIMIE DE MONTPELLIER, MONTPELLIER CEDEX 5 (FR)

(21) Appl. No.: 18/852,573

(22) PCT Filed: Mar. 31, 2023
(86) PCT No.: PCT/FR2023/050471

§ 371 Date: Sep. 30, 2024

(30) Foreign Application Priority Data
Mar. 31, 2022 (FR) 2202962

(51) Int. Cl. C08J 3/18; C07C 67/08; C07D 303/42; C08J 5/18; C08K 5/134; C08K 5/1515

(52) U.S. Cl. CPC C08J 3/18; C07C 67/08; C07D 303/42; C08J 5/18; C08K 5/1345; C08K 5/1515; C08J 2301/12

(57) ABSTRACT

The use of a composition including at least one compound derived from cashew nut shell oil (CNSL), which has at least one ester function and/or at least one epoxide function, as a plasticizing agent for cellulose acetate, for the preparation of a plastic material including or solely composed of cellulose acetate.

The present invention concerns new compounds in the field of plastic materials, as plasticizing agents, in particular cellulose acetate. The invention also concerns a process for preparing such plastic materials.

### BACKGROUND TO THE INVENTION

Plastic materials are mostly of petroleum origin. In most cases, they are non-biodegradable, leading to plastic pollution in the oceans and problematic management in terms of recycling.

Today's consumers are increasingly concerned about ecological and health issues, and therefore expect environmentally-friendly materials that are not harmful to health.

Against this backdrop, plastics with plant-based components are now on the market, such as biosourced plastics, most of which come from wheat, corn, sugar beet, sugar cane or potatoes, i.e. from intensive crops whose production competes with human food supplies.

There is therefore a need to provide consumers with alternative plastic materials that are sustainably biosourced, environmentally friendly and biodegradable, with thermoplastic properties equivalent to those of plastics derived from the petroleum industry.

Cellulose is one of the most abundant biopolymers. Substitution of cellulose hydroxyl groups by other functional groups is essential to obtain thermoplastic properties. Cellulose derivatives are characterized by their degree of substitution (DS), i.e. the average number of substituent groups present per anhydroglucopyranose unit (AGU). The degree of substitution can vary from 0 (no hydroxyl groups are replaced) to 3 (all hydroxyl groups are replaced).

Cellulose acetate corresponds to substitution by acetyl groups (—COCH₃). Due to its multiple properties, cellulose acetate is one of the most common cellulose derivatives used in industry. It is a transparent, glossy, non-flammable and tough polymer, with good dimensional stability and high resistance to heat and chemicals. The formula for cellulose acetate with a DS of 2.0 is as follows, where n represents the number of monomers, i.e. the degree of polymerization:

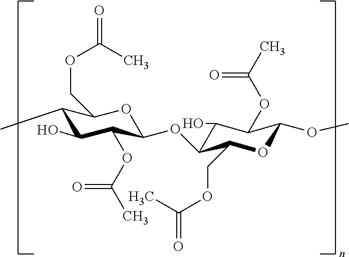

To meet the desired technical properties of plastics, additives are often added to cellulose acetate. Among additives, plasticizers are small organic molecules that reduce brittleness and improve flexibility by reducing crystallinity and by lowering glass transition and melting temperatures.

The ideal plasticizer must be able to significantly reduce the glass transition temperature (Tg), be biodegradable, non-volatile and non-toxic, and migrate only slightly over time.

To achieve effective plasticization, the compatibility between plasticizer and polymer is a major factor. Indeed, if the plasticizer and polymer are highly compatible, this can lead to the establishment of bonds that would stiffen the material. However, they must be sufficiently compatible to prevent the plasticizer from migrating prematurely.

In biopolymer-based films, incompatibility generally arises, as evidenced by biopolymer/plasticizer phase separation, presented in the form of droplets exuded from the film surface.

Cashew Nut Shell Oil or CNSL is a natural oil derived from cashew nut shells. The main components of crude CNSL are phenolic compounds: anacardic acid, cardol and cardanol. Methyl cardol is also present, but in trace amounts. Each of these compounds is itself a mixture of products, substituted by an alkyl or alkenyl chain, said alkenyl chain possessing 1, 2 or 3 double bonds (Scheme 1):

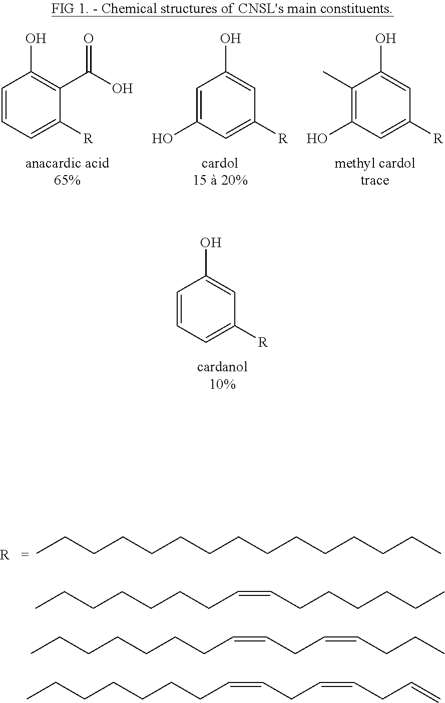

CNSL can be classified into 2 types, depending on the extraction method used:
CNSL extract, obtained by solvent extraction,
Technical CNSL, obtained by hot processes, in particular high-temperature roasting, e.g. >250° C.

To date, CNSL oil has yet to be valorized, given the potential of its constituent components.

One of the aims of the invention is to use compounds derived from natural products as plasticizing agents for biopolymers.

Another aim of the invention is to use compounds derived from plant sources as plasticizing agents, enabling compatibility with biopolymers, in order to obtain biodegradable plastic materials.

Another aim of the invention is the use of compounds obtained from natural compounds contained in agricultural production waste for their valorization.

Yet another aim of the present invention is to provide a plastic material from biobased materials that does not come from intensive crops whose production competes with human food.

Yet another aim of the invention is a process for preparing such plastic materials.

A first object of the present invention is the use of a composition comprising at least one CNSL-derived compound which has at least one ester function and/or at least one epoxide function, as a plasticizing agent for cellulose acetate, for the preparation of a plastic material comprising or consisting of cellulose acetate.

The Inventors have surprisingly found that compounds derived from cashew nut shells, which are currently little or not valorized, as well as products inspired by these compounds, with similar structures, can be transformed into derivatives with interesting properties as plasticizing agents.

The phenolic compounds present in Cashew Nut Shell Oil (CNSL) can be esterified and/or peroxidized to produce compounds that can be used as plasticizers for cellulose acetate to form plastic materials.

Technical CNSL contains a reduced amount of anacardic acid, due to partial decarboxylation during roasting. In some cases, decarboxylation is even total, and CNSL no longer contains anacardic acid.

By “raw, non-decarboxylated CNSL” it is meant a CNSL that has not undergone, or has only partially undergone, decarboxylation of anacardic acid. Raw, non-decarboxylated CNSL is therefore characterized by the presence of anacardic acid in the mixture.

The CNSL, a cashew nut shell oil, used in the present invention is preferably a “crude CNSL”, having undergone no decarboxylation, or a CNSL having undergone only partial decarboxylation of anacardic acid.

In other words, the CNSL used in the present invention is preferably a “CNSL” comprising at least 5% by weight of anacardic acid, or in which the sum of the percentage of anacardic acid, cardol, and methyl cardol is at least 15% by weight.

By “at least 5% by weight of anacardic acid” it is also meant: at least 10% by weight, at least 20% by weight, at least 30% by weight, at least 40% by weight, at least 50% by weight, preferably at least 60% by weight.

By “the sum of the percentage of anacardic acid, cardol and methyl cardol is at least 15% by mass” it is also meant: at least 25% by mass, at least 35% by mass, at least 45% by mass, at least 55% by mass, at least 65% by mass, preferably at least 75% by mass.

By “CNSL derivative” it is meant a compound, or compounds, obtained from CNSL.

Indeed, it is possible to isolate anacardic acid or cardol, or methyl cardol, or cardanol in order to provide “pure” CNSL-derived compounds.

In this context, CNSL can be used as a mixture which is converted into derivative compounds of interest, possibly followed by separation and purification.

By “CNSL derivative containing at least one ester function”, it is meant a CNSL derivative compound containing an ester group, i.e. a group of formula —CO—OR formed by a carbon atom linked simultaneously to an oxygen atom by a double bond and to an alkoxy group, i.e. an oxygen linked to a carbon chain.

By “CNSL derivative with at least one epoxide function” it is meant a CNSL-derived compound with an epoxide group along a carbon chain, i.e. a group of a bridged oxygen on a carbon-carbon bond.

By “plasticizing agent” it is meant an additive in the form of chemical compounds capable of creating interactions with cellulose acetate and modifying the properties of the biopolymer.

There are two main categories of plasticizing agents:
external plasticizers, which are incorporated as an additive into a plastic compound, without the existence of covalent bonds between the polymer and the plasticizing agent; they can therefore be lost through evaporation, migration or extraction;
internal plasticizers, more advantageous, are chemical groups incorporated by chemical reaction or grafting into a polymer with the aim of plasticizing it; internal plasticizers generally have bulky structures providing bulkier structural materials sufficient to prevent polymer chains from drawing together.

The material obtained after plasticization is generally amorphous and can be processed in conventional material shaping processes over a temperature range from glass transition to degradation.

Advantageously in the present invention, the cellulose acetate used is preferably a cellulose acetate with a degree of substitution DS varying from 1.5 to 3.0, preferably 2.0 to 2.8, in particular 2.5.

The “1.5 to 3.0” range includes the ranges: from 1.5 to 1.6; from 1.6 to 1.7; from 1.7 to 1.8; from 1.8 to 1.9; from 1.9 to 2.0; from 2.0 to 2.1; from 2.1 to 2.2; from 2.2 to 2.3; from 2.3 to 2.4; from 2.4 to 2.5; from 2.5 to 2.6; from 2.6 to 2.7; from 2.7 to 2.8; from 2.8 to 2.9; from 2.9 to 3.0.

By “plastic material” it is meant a material consisting of a polymer matrix mixed with additives, colorants or fillers, with thermoplastic properties that enable it to be shaped, and with properties of strength, transparency and lightness.

Plastic materials can take the form of films, fabrics or objects of defined shape.

By “plastic material comprising cellulose acetate” it is meant that in said plastic material, part of the polymer matrix is based on cellulose acetate.

By “plastic material consisting of cellulose acetate” it is meant that the polymer matrix is made from cellulose acetate without any other plastic material, and also includes additives, colorants or fillers.

According to another particular embodiment, the present invention relates to the use as defined above, of a composition in which the CNSL-derived compound comprises at least one ester function and does not comprise an epoxide function, as plasticizing agent for cellulose acetate, for the preparation of a plastic material comprising or consisting of cellulose acetate.

According to another particular embodiment, the present invention relates to the use as defined above, of a composition in which the CNSL-derived compound comprises an epoxide function and does not comprise an ester function for the preparation of a plastic material comprising or consisting of cellulose acetate.

According to another particular embodiment, the present invention relates to the use as defined above, of a composition in which the CNSL-derived compound comprises at least one ester function and comprises at least one epoxide function, as plasticizing agent for cellulose acetate, for the preparation of a plastic material comprising or consisting of cellulose acetate.

According to another particular embodiment, the present invention relates to the use as defined above of a composition comprising at least one CNSL-derived compound which has at least one ester function and/or at least one epoxide function, as a primary plasticizing agent for cellulose acetate, for the preparation of a plastic material comprising or consisting of cellulose acetate.

The term “primary plasticize” designates a molecule that lowers the glass transition temperature and modulus of elasticity of a polymer when added without other plasticizers. It is chemically compatible with the polymer it plasticizes, enabling it to be solubilized up to 30-60% by weight of the total material weight.

A primary plasticizer is defined as opposed to a secondary plasticizer, which on its own cannot sufficiently reduce glass transition temperature and modulus of elasticity. Its properties depend on the application for which the material is being developed. A secondary plasticizer requires the use of a primary plasticizer to form a plastic material. The compatibility of the secondary plasticizer with the polymer is significantly lower than that of the primary plasticizer. In particular, this incompatibility can lead to exudation of the secondary plasticizer, and this phenomenon can be avoided by adding the primary plasticizer.

According to another particular embodiment, the present invention relates to the use as defined above of a composition comprising at least one compound derived from CNSL which has at least one ester function and/or at least one epoxide function and does not have linear ether functions, as a primary plasticizing agent for cellulose acetate, for the preparation of a plastic material comprising or consisting of cellulose acetate.

According to another particular embodiment, the present invention relates to the use as defined above of a composition comprising cardol, as a primary plasticizing agent for cellulose acetate, for the preparation of a plastic material comprising or consisting of cellulose acetate.

According to another particular embodiment, the present invention relates to the use as defined above of a composition, comprising at least one compound of formula (1) as a primary plasticizing agent of cellulose acetate for the preparation of a plastic material comprising or consisting of cellulose acetate,

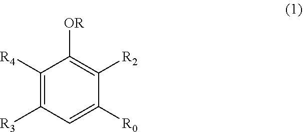

formula (1) in which
R is selected from
   a hydrogen atom or
   a CO—R₁ group,
R₀ is selected from
   a linear alkyl group of 15 carbon atoms, optionally comprising from 1 to 3 epoxide functions or,
   a linear alkenyl group of 15 carbon atoms, comprising x alkenyl functions, x varying from 1 to 3, comprising y epoxide functions, y varying from 0 to 2, x+y being less than or equal to 3,
R₂ is selected from
   a hydrogen atom,
   a COOH carboxylic acid function,
   CO—O—R₅ of formula

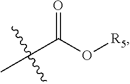

R₃ is selected from
   a hydrogen atom,
   an alcohol function —OH
   —OC—O—R₆ with formula

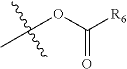

R₄ is selected from
   a hydrogen atom or
   a methyl group CH₃;
R₁, R₅ and R₆ are identical or different and are selected from
   a linear alkyl group of 1 to 30 carbon atoms, optionally containing 1 to 6 epoxide functions, or
   a linear alkenyl group of 2 to 24 carbon atoms, optionally comprising 1 to 6 epoxide functions, said alkenyl group being optionally substituted by an alkyl group of 1 to 5 carbon atoms,
      R₀, R₁, R₅ and R₆ optionally comprising one to six substituent(s) selected from —OH, —OR₇ or OCOR₇, wherein R₇ is an alkyl group of 1 to 3 carbon atoms,
      with the proviso that at least one of the OR, R₂ or R₃ groups is an ester function and/or at least one of R₀, R₁, R₅ or R₆ comprises at least one epoxide function.
      or at least one of the compounds of the following formulae:

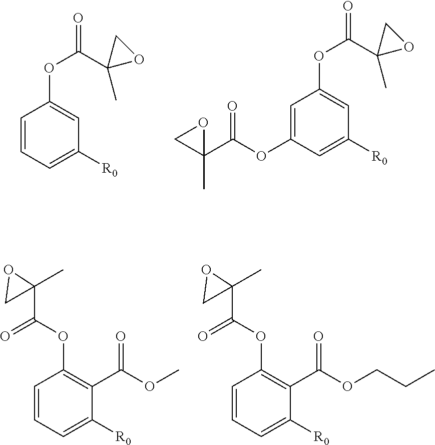

By “epoxide function” it is meant a group of an oxygen bridged on a carbon-carbon bond.

By “alkenyl function” it is meant an unsaturated C═C group, i.e. at least one carbon-carbon double bond.

It is understood that x and y are positive integers and that x indicates the number of epoxide functions present in the R₀ group and y indicates the number of epoxide functions present in the R₀ group. The number of epoxide or alkenyl functions present in the R₀ group cannot be greater than 3.

When y is 0, there is no epoxide group on the R₀ group.

By “linear alkyl group of 1 to 30 carbon atoms” it is meant a carbon chain with 1 to 30 carbon atoms.

By “optionally comprising from 1 to 6 epoxide functions”, it is meant that the carbon chain comprises from 0 to 6 epoxide functions. In other words, the carbon chain either comprises no epoxide functions or comprises from 1 to 6 oxygen atom(s) bridged on the carbon atoms of the carbon chain.

By “linear alkenylgroup of 2 to 24 carbon atoms” it is meant a linear carbon chain with 2 to 24 carbon atoms comprising at least one alkenyl function, i.e. at least one carbon-carbon double bond(s).

By “alkyl group of 1 to 5 carbon atom(s)” it is meant a linear or branched alkyl group of 1 to 5 carbon atom(s). These groups include methyl, ethyl, propyl, butyl and pentyl. The definition of propyl, butyl, pentyl, hexyl, heptyl, octyl, nonyl and decyl includes all possible isomers. For example, the term butyl includes n-butyl, iso-butyl, sec-butyl and ter-butyl.

By “alkylgroup of 1 to 3 carbon atom(s)” it is meant a linear or branched alkyl group comprising 1 to 3 carbon atom(s). These include methyl, ethyl, propyl and isopropyl groups.

According to another particular embodiment, the present invention relates to the use as defined above of a composition comprising at least one compound of formula (1) as a primary plasticizing agent of cellulose acetate for the preparation of a plastic material comprising or consisting of cellulose acetate,

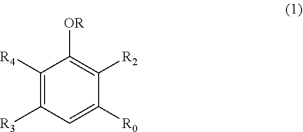

formula (1) in which
R is selected from
   a hydrogen atom or
   a CO—R₁ group,
R₀ is selected from
   a linear alkyl group of 15 carbon atoms, optionally comprising from 1 to 3 epoxide functions or,
   a linear alkenyl group of 15 carbon atoms, comprising x alkenyl functions, x varying from 1 to 3, comprising y epoxide functions, y varying from 0 to 2, x+y being less than or equal to 3,
R₂ is selected from
   a hydrogen atom,
   a COOH carboxylic acid function,
   —CO—O—R₅ with formula

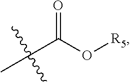

R₃ is selected from
   a hydrogen atom,
   an alcohol function —OH
   —OC—O—R₆ with formula

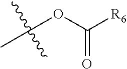

R₄ is selected from
   a hydrogen atom or
   a methyl group CH₃;
R₁, R₅ and R₆ are identical or different and are selected from
   a linear alkyl group of 1 to 30 carbon atoms, optionally containing 1 to 6 epoxide functions, or
   a linear alkenyl group of 2 to 24 carbon atoms, optionally comprising 1 to 6 epoxide functions, said alkenyl group being optionally substituted by an alkyl group of 1 to 5 carbon atoms,
      R₀ optionally comprising one to six substituent(s) selected from —OH, —OR₇ or OCOR₇, wherein R₇ is an alkyl group of 1 to 3 carbon atoms,
      R₁, R₅ and R₆ optionally comprising one or two substituents selected from —OH, —OR₇ or OCOR₇,
      with the proviso that at least one of the OR, R₂ or R₃ groups is an ester function and/or at least one of R₀, R₁, R₅ or R₆ comprises at least one epoxide function.
      or at least one of the compounds of the following formulae:

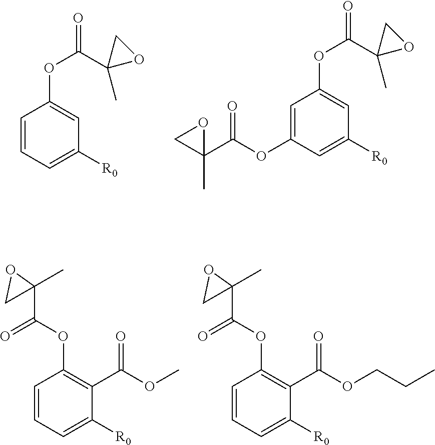

According to another particular embodiment, the present invention relates to the use as defined above, of a composition comprising at least one compound of formula (1), as cellulose acetate plasticizing agent for the preparation of a plastic material comprising or consisting of cellulose acetate,

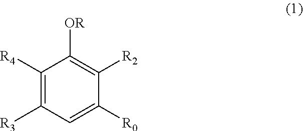

formula (1) in which
R is selected from
   a hydrogen atom or
   a CO—R₁ group,
R₀ is selected from
   a linear alkyl group of 15 carbon atoms, optionally comprising from 1 to 3 epoxide functions or,
   a linear alkenyl group of 15 carbon atoms, comprising x alkenyl functions, x varying from 1 to 3, comprising y epoxide functions, y varying from 0 to 2, x+y being less than or equal to 3,
R₂ is selected from
   a hydrogen atom,
   —CO—O—R₅ with formula

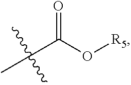

a COOH carboxylic acid function,
R₃ is selected from
   a hydrogen atom,
   an alcohol function —OH,
   —OC—O—R₆ formula,

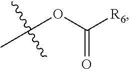

R₄ is selected from
   a hydrogen atom or
   a methyl group CH₃;
R₁, R₅ and R₆ are identical or different and are selected from
   a linear alkyl group of 1 to 30 carbon atoms, optionally containing 1 to 6 epoxide functions, or
   a linear alkenyl group of 2 to 24 carbon atoms, optionally comprising 1 to 6 epoxide functions, said alkenyl group being optionally substituted by an alkyl group of 1 to 5 carbon atoms,
      R₁, R₅ and R₆ optionally comprising one or two substituents selected from —OH, —OR₇ or OCOR₇, wherein R₇ is an alkyl group of 1 to 3 carbon atoms,
      with the proviso that at least one of the OR, R₂ or R₃ groups is an ester function and/or at least one of the R₀, R₁, R₅ or R₆ groups comprises at least one epoxide function.

According to another particular embodiment, the present invention relates to the use as defined above, of a composition comprising at least one compound of formula (1), as a primary plasticizing agent of cellulose acetate for the preparation of a plastic material comprising or consisting of cellulose acetate,

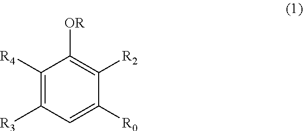

formula (1) in which
R is selected from
   a hydrogen atom or
   a CO—R₁ group,
R₀ is selected from
   a linear alkyl group of 15 carbon atoms, optionally comprising from 1 to 3 epoxide functions or,
   a linear alkenyl group of 15 carbon atoms, comprising x alkenyl functions, x varying from 1 to 3, comprising y epoxide functions, y varying from 0 to 2, x+y being less than or equal to 3,
R₂ is selected from
   a hydrogen atom,
   —CO—O—R₅ with formula

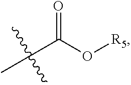

a COOH carboxylic acid function,
R₃ is selected from
   a hydrogen atom,
   an alcohol function —OH,
   —OC—O—R₆ formula,

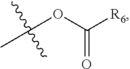

R₄ is selected from
   a hydrogen atom or
   a methyl group CH₃;
R₁, R₅ and R₆ are identical or different and are selected from
   a linear alkyl group of 1 to 30 carbon atoms, optionally containing 1 to 6 epoxide functions, or
   a linear alkenyl group of 2 to 24 carbon atoms, optionally comprising 1 to 6 epoxide functions, said alkenyl group being optionally substituted by an alkyl group of 1 to 5 carbon atoms,
      R₁, R₅ and R₆ optionally comprising one or two substituents selected from —OH, —OR₇ or OCOR₇, wherein R₇ is an alkyl group of 1 to 3 carbon atoms,
      with the proviso that at least one of the OR, R₂ or R₃ groups is an ester function and/or at least one of the R₀, R₁, R₅ or R₆ groups comprises at least one epoxide function.

According to another particular embodiment, the present invention relates to the use as defined above, of a composition comprising at least one compound of formula (1b), as a primary plasticizing agent of cellulose acetate for the preparation of a plastic material comprising or consisting of cellulose acetate,

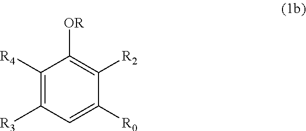

formula (1b) in which
R is selected from
   a hydrogen atom,
   a CO—R₁ group,
R₀ is selected from
   a linear alkyl group of 15 carbon atoms optionally containing 1 to 3 epoxide functions, or
   a linear alkenyl group of 15 carbon atoms, comprising x alkenyl functions, x varying from 1 to 3, comprising y epoxide functions, y varying from 0 to 2, x+y being less than or equal to 3,
R₂ is selected from
   a hydrogen atom,
   —CO—O—R₅ with formula

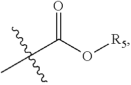

a COOH carboxylic acid function,
R₃ is selected from
   a hydrogen atom,
   —OC—O—R₆ with formula

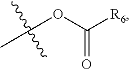

an alcohol function —OH,
R₄ is selected from
   a hydrogen atom,
   a methyl group CH₃;
R₁, R₅ and R₆ are identical or different and are selected from
   a linear alkyl group of 1 to 30 carbon atom(s), or
   a linear alkenyl group of 2 to 24 carbon atoms, comprising 1 to 6 alkenyl function(s), said alkenyl group being optionally substituted by a linear alkyl group of 1 to 5 carbon atom(s),
      R₀ optionally comprising one to six substituent(s) selected from —OH, —OR₇ or OCOR₇, wherein R₇ is an alkyl group of 1 to 3 carbon atoms,
      R₁, R₅ and R₆ optionally comprising one to six substituent(s), in particular one or two, selected from —OH, —OR₇ or OCOR₇,
      with the proviso that at least one of the OR, R₂ or R₃ groups is an ester function.

According to another particular embodiment, the present invention relates to the use, as defined above, of a composition comprising at least one compound of formula (1b), in which the group R₀ does not comprise an epoxide function.

According to another particular embodiment, the present invention relates to the use as defined above, of a composition comprising at least one compound of formula (1d), as a primary plasticizing agent of cellulose acetate for the preparation of a plastic material comprising or consisting of cellulose acetate,

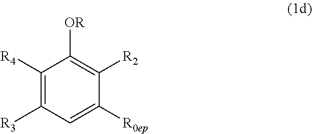

formula (1d) in which:
R is selected from
   a hydrogen atom
   a CO—R₁ group,
R₀ₑₚ is selected from
   a linear alkyl group of 15 carbon atoms optionally containing 1 to 3 epoxide functions, or
   a linear alkenyl group of 15 carbon atoms, comprising x alkenyl functions, x varying from 1 to 3, optionally comprising y epoxide functions, y varying from 0 to 2, x+y being less than or equal to 3,
R₀ₑₚ optionally comprising one to six substituent(s) selected from —OH, OR₇ or OCOR₇, wherein R₇ is an alkyl group of 1 to 3 carbon atoms
R₂ is selected from
   a hydrogen atom,
   a COOH carboxylic acid function, or
   —CO—O—R₅ with formula

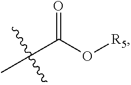

R₃ is selected from
   a hydrogen atom,
   an OH alcohol function, or
   —OC—O—R₆ with formula

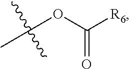

R₄ is selected from
   a hydrogen atom,
   a methyl group CH₃,
R₁, R₅ and R₆ are identical or different and are selected from
   a linear alkyl group of 1 to 30 carbon atom(s), optionally containing 1 to 6 epoxide function(s), or
   a linear alkenyl group of 2 to 24 carbon atoms, optionally comprising 1 to 6 epoxide function(s), said alkenyl group being optionally substituted by an alkyl group of 1 to 5 carbon atom(s),
      R₁, R₅ and R₆ optionally comprising one or two substituents selected from —OH, —OR₇ or OCOR₇, wherein R₇ is an alkyl group of 1 to 3 carbon atoms,
      with the proviso that at least one of the OR, R₂ or R₃ groups is an ester function and at least one of the R₀ₑₚ, R₁, R₅ or R₆ groups comprises at least one epoxide function.

According to another particular embodiment, the present invention relates to the use as defined above, of a composition comprising at least one compound of formula (1d), as a primary plasticizing agent of cellulose acetate for the preparation of a plastic material comprising or consisting of cellulose acetate,

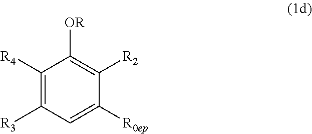

formula (1d) in which
R is selected from
   a hydrogen atom
   a CO—R₁ group,
R₀ₑₚ is selected from
   a linear alkyl group of 15 carbon atoms optionally containing 1 to 3 epoxide functions, or
   a linear alkenyl group of 15 carbon atoms, comprising x alkenyl functions, x varying from 1 to 3, optionally comprising y epoxide functions, y varying from 0 to 2, x+y being less than or equal to 3
R₂ is selected from
   a hydrogen atom,
   a COOH carboxylic acid function, or
   —CO—O—R₅ with formula

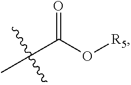

R₃ is selected from
   a hydrogen atom,
   an OH alcohol function, or
   —OC—O—R₆ with formula

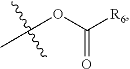

R₄ is selected from
   a hydrogen atom,
   a methyl group CH₃,
R₁, R₅ and R₆ are identical or different and are selected from:
   a linear alkyl group of 1 to 30 carbon atom(s), optionally containing 1 to 6 epoxide function(s), or
   a linear alkenyl group of 2 to 24 carbon atoms, optionally comprising 1 to 6 epoxide function(s), said alkenyl group being optionally substituted by an alkyl group of 1 to 5 carbon atom(s),
      R₀ₑₚ optionally comprising one to six substituent(s) selected from —OH, —OR₇ or OCOR₇, wherein R₇ is an alkyl group of 1 to 3 carbon atoms,
      R₁, R₅ and R₆ optionally comprising one to six substituent(s), in particular one or two, selected from —OH, —OR₇ or OCOR₇,
      with the proviso that at least one of the OR, R₂ or R₃ groups is an ester function and at least one of the R₀ₑₚ, R₁, R₅ or R₆ groups comprises at least one epoxide function.

According to another particular embodiment, the present invention relates to the use as defined above, of a composition in which the compound of formula (1b) is selected from compounds of the following formulae (2), (3), (4), (5) and (6),

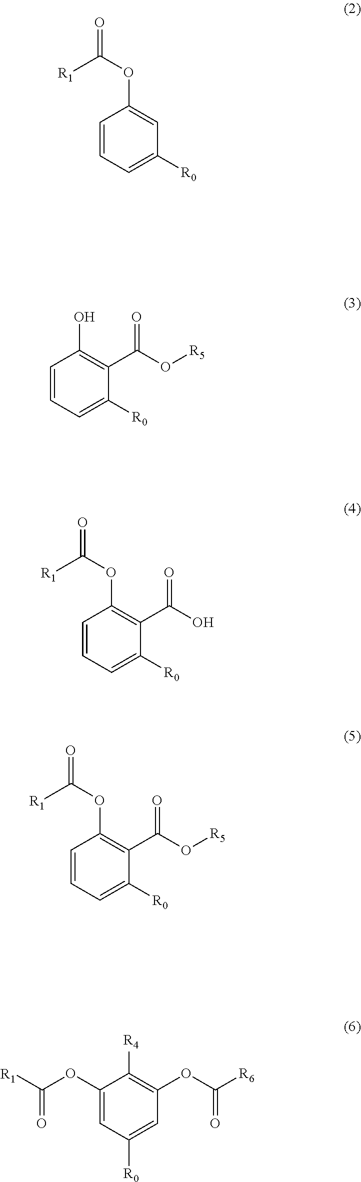

in which R₀ is selected from:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3.
   R₄ is selected from:
a hydrogen atom, or
a methyl group CH₃
   R₁, R₅ and R₆ are selected from
a linear alkyl group of 1 to 30 carbon atom(s), or
a linear alkenyl group of 2 to 24 carbon atoms, said alkenyl group being optionally substituted by an alkyl group of 1 to 5 carbon atoms,
said R₀ group optionally comprising one to six substituent(s) selected from —OH, —OR₇ or OCOR₇, wherein R₇ is an alkyl group of 1 to 3 carbon atom(s)
said R₁, R₅ and R₆ groups optionally comprising one to six substituent(s), in particular one or two, selected from —OH, —OR₇ or OCOR₇.

According to another particular embodiment, the present invention relates to the use as defined above, of a composition in which the compound of formula (1c) is selected from compounds of the following formulae (7), (8), and (9),

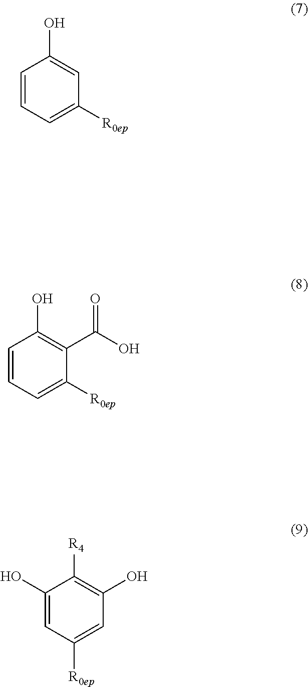

in which R₀ₑₚ is selected from:
a linear alkyl group of 15 carbon atoms containing from 1 to 3 epoxide functions, or
a linear alkenyl group of 15 carbon atoms, comprising x alkenyl functions, x varying from 1 to 2, comprising y epoxide functions, y varying from 1 to 2, x+y being less than or equal to 3
R₀ₑₚ optionally comprising one to six substituent(s) selected from —OH, OR₇ or OCOR₇, wherein R₇ is an alkyl group of 1 to 3 carbon atoms

According to another particular embodiment, the present invention relates to the use as defined above, of a composition in which the compound of formula (1d) is selected from compounds of the following formulae (10), (11), (12), (13) and (14),

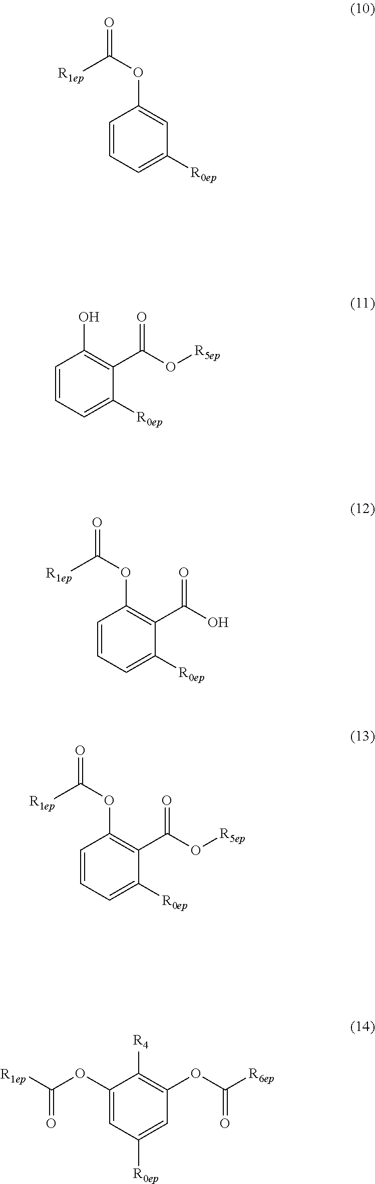

in which R₀ₑₚ is selected from:
   a linear alkyl group of 15 carbon atoms optionally containing 1 to 3 epoxide functions, or
   a linear alkenyl group of 15 carbon atoms, comprising x alkenyl functions, x varying from 1 to 3, comprising y epoxide functions, y varying from 0 to 2, x+y being less than or equal to 3
R₄ is selected from
   a hydrogen atom,
   a methyl group CH₃
R₁ₑₚ, R₅ₑₚ and R₆ₑₚ are selected from
   a linear alkyl group of 1 to 30 carbon atom(s), optionally containing 1 to 6 epoxide function(s), or
   a linear alkenyl group of 2 to 24 carbon atoms, optionally comprising 1 to 6 epoxide function(s), said alkenyl group being optionally substituted by an alkyl group of 1 to 5 carbon atom(s),
said R₀ₑₚ group optionally comprising one to six substituent(s) selected from —OH, —OR₇ or OCOR₇, wherein R₇ is an alkyl group of 1 to 3 carbon atom(s), said R₁ₑₚ, R₅ₑₚ and R₆ₑₚ groups optionally comprising one to six substituent(s), in particular one or two, selected from —OH, —OR₇ or OCOR₇,
   with the proviso that at least one of the R₀ₑₚ, R₁ₑₚ, R₅ₑₚ or R₆ₑₚ groups contains at least one epoxide function.

It is understood that:
in formula (10), at least one of R₀ₑₚ or R₁ₑₚ comprises at least one epoxide function,
in formula (11), at least one of R₀ₑₚ or R₅ₑₚ comprises at least one epoxide function,
in formula (12), at least one of R₀ₑₚ or R₁ₑₚ comprises at least one epoxide function,
in formula (13), at least one of R₀ₑₚ, R₁ₑₚ or R₅ₑₚ comprises at least one epoxide function,
in formula (14), at least one of R₀ₑₚ, R₁ₑₚ or R₆ₑₚ comprises at least one epoxide function.

According to another particular embodiment, the present invention relates to the use as defined above, of a composition in which the compound of formula (1b) is chosen from compounds of formulae (2), (3), (4), (5) and (6) in which:
R₀ is selected from

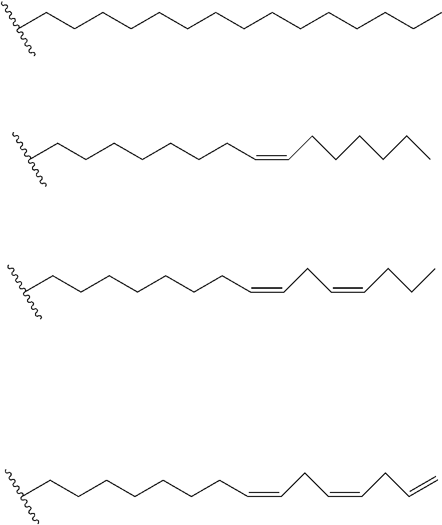 [SMILES: *CCCCCCCCCCCCCCC ; *CCCCCCC/C=C\CCCCCC ; *CCCCCCC/C=C\C/C=C\CCC ; *CCCCCCC/C=C\C/C=C\CC=C]

R₁, R₅ and R₆ are independently selected from:
   an alkyl group of 1 to 30 carbon atoms,
   a linear alkenyl group of 2 to 24 carbon atoms, said alkenyl group being optionally substituted by an alkyl chain with 1 to 5 carbon atoms,
      said R₀ group optionally comprising one to six substituent(s) selected from —OH, —OR₇ or OCOR₇, wherein R₇ is an alkyl group of 1 to 3 carbon atom(s)
      said R₁, R₅ and R₆ groups optionally comprising one to six substituent(s), in particular one or two, selected from —OH, —OR₇ or OCOR₇,
      with the proviso that at least one of the OR, R₂ or R₃ groups is an ester function.

It is understood that the left end of the R₀ group is linked to the phenolic group and the right end of the R₀ group is free.

According to another particular embodiment, the present invention relates to the use as defined above, of a composition in which the compound of formula (1c) is chosen from compounds of formula (7), (8) and (9), in which:
R₀ₑₚ is selected from

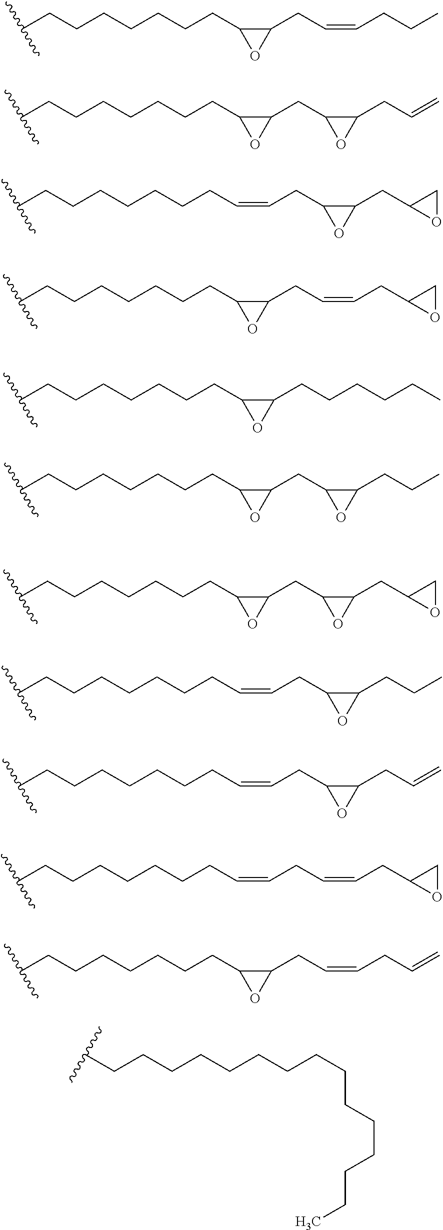

said R₀ₑₚ group optionally comprising 1 to 6 substituent(s) selected from —OH, —OR₇ and OCOR₇

It is understood that the left end of the R₀ₑₚ group is linked to the phenolic group and the right end of the R₀ₑₚ group is free.

According to another particular embodiment, the present invention relates to the use as defined above, of a composition in which the compound of formula (1d) is chosen from compounds of formulae (10), (11), (12), (13) or (14) in which:
R₀ₑₚ is selected from:

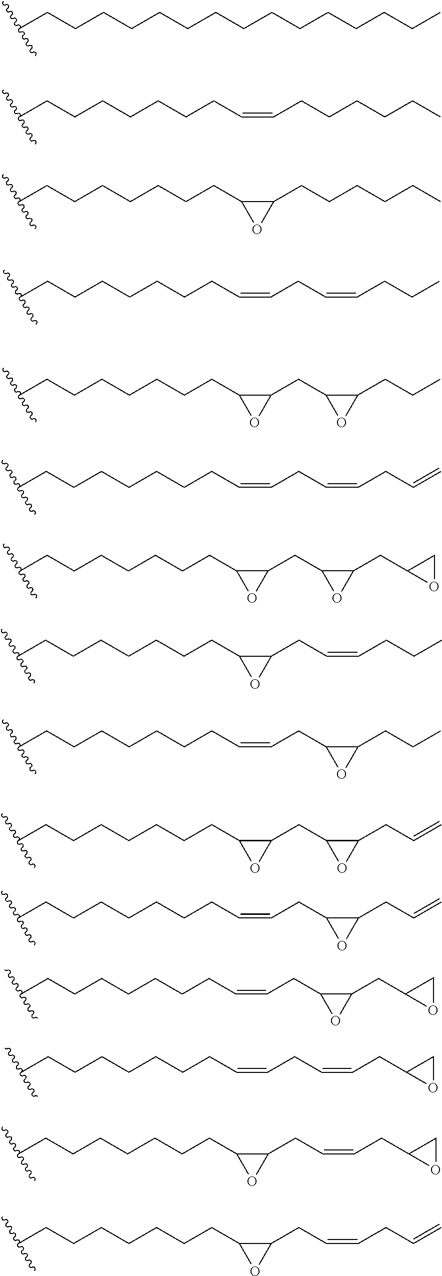

R₁ₑₚ, R₅ₑₚ and R₆ₑₚ are independently selected from:
   an alkyl group of 1 to 30 carbon atoms, optionally comprising 1 to 6 epoxide functions,
   a linear alkenyl group of 2 to 24 carbon atoms, in particular comprising 1 to 6 epoxide functions, said alkenyl group being optionally substituted by an alkyl chain with 1 to 5 carbon atoms,
      said R₀ₑₚ group optionally comprising one to six substituent(s) selected from —OH, —OR₇ or OCOR₇, wherein R₇ is an alkyl group of 1 to 3 carbon atom(s),
      said R₁ₑₚ, R₅ₑₚ and R₆ₑₚ groups optionally comprising one to six substituent(s), in particular one or two, selected from —OH, —OR₇ or OCOR₇,
      with the proviso that at least one of the R₀ₑₚ, R₁ₑₚ, R₅ₑₚ or R₆ₑₚ groups contains at least one epoxide function.

According to another particular embodiment, the present invention relates to the use as defined above, of a composition in which the compound of formula (1) is selected from compounds of the following formulae:

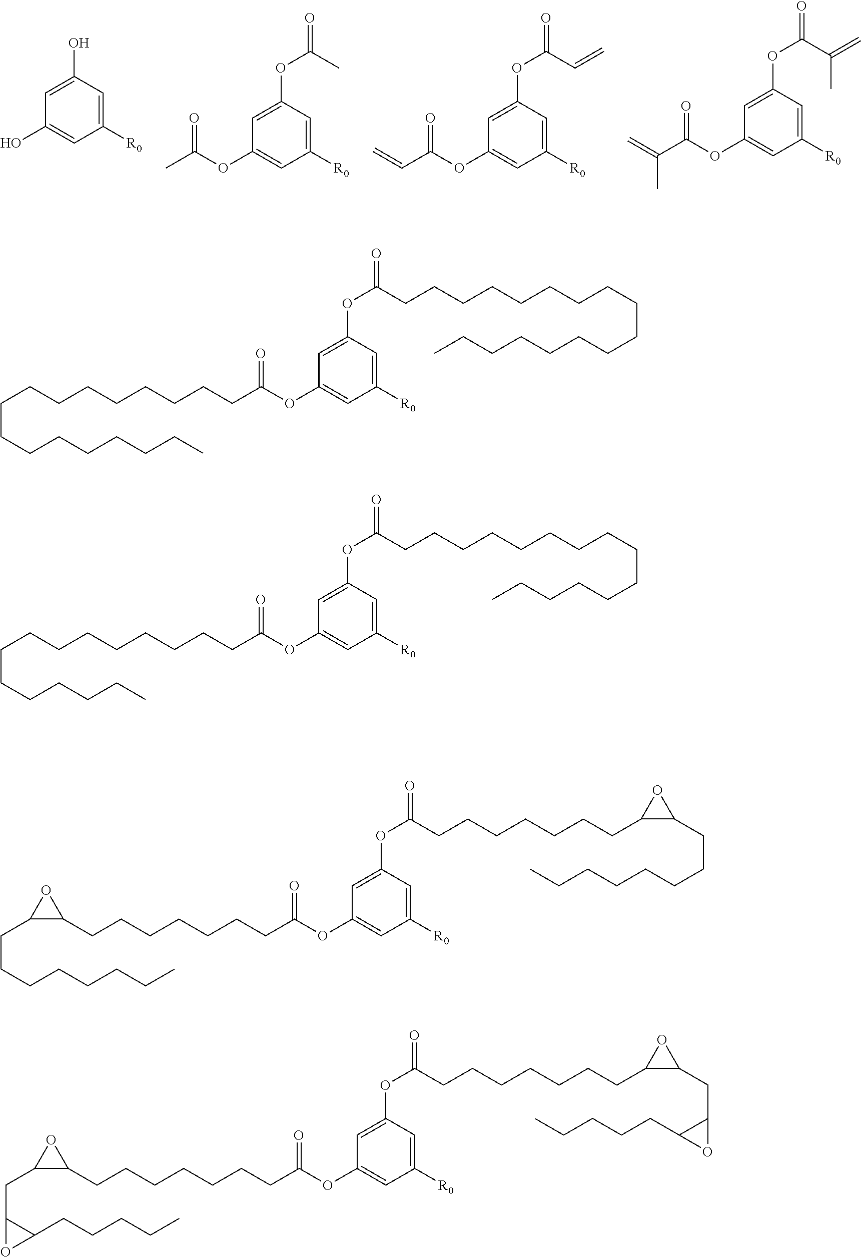

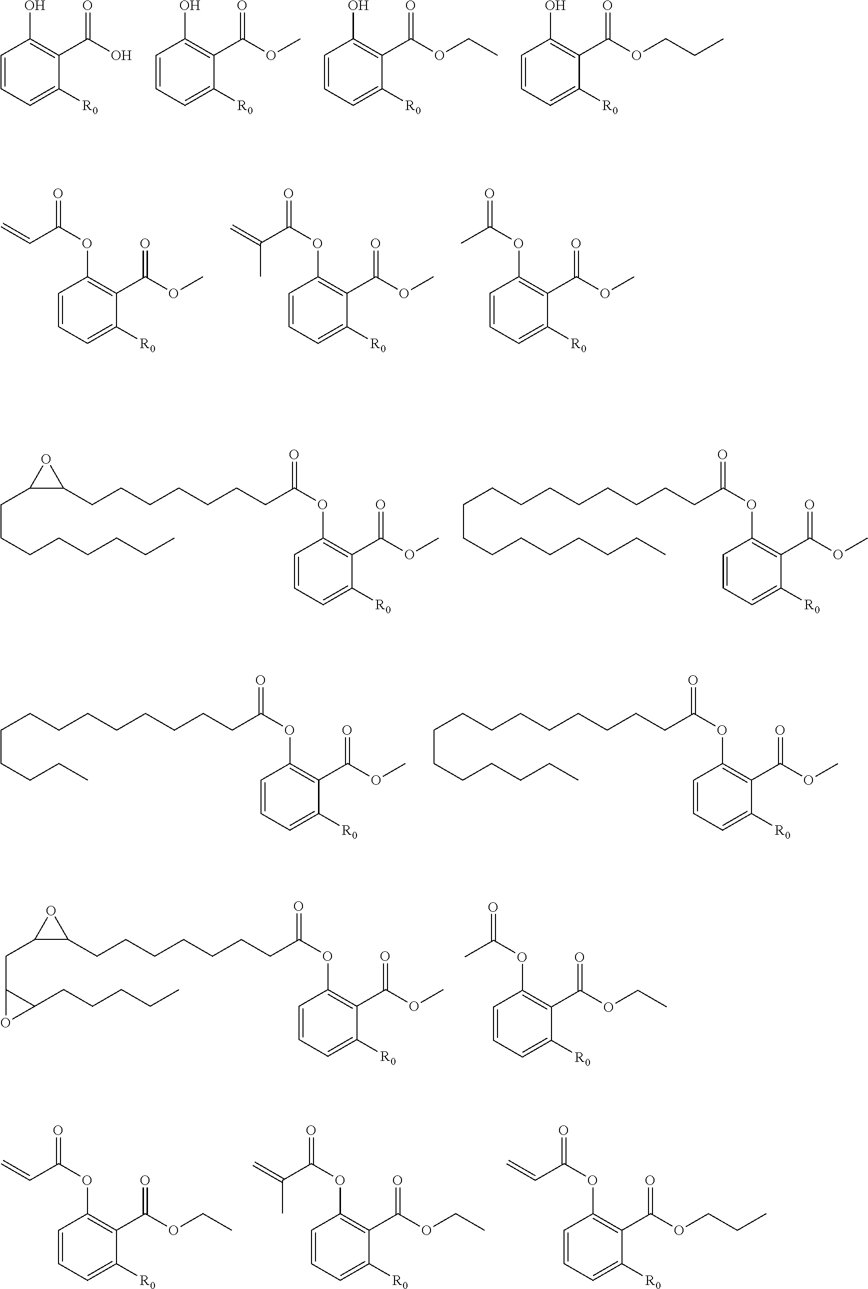

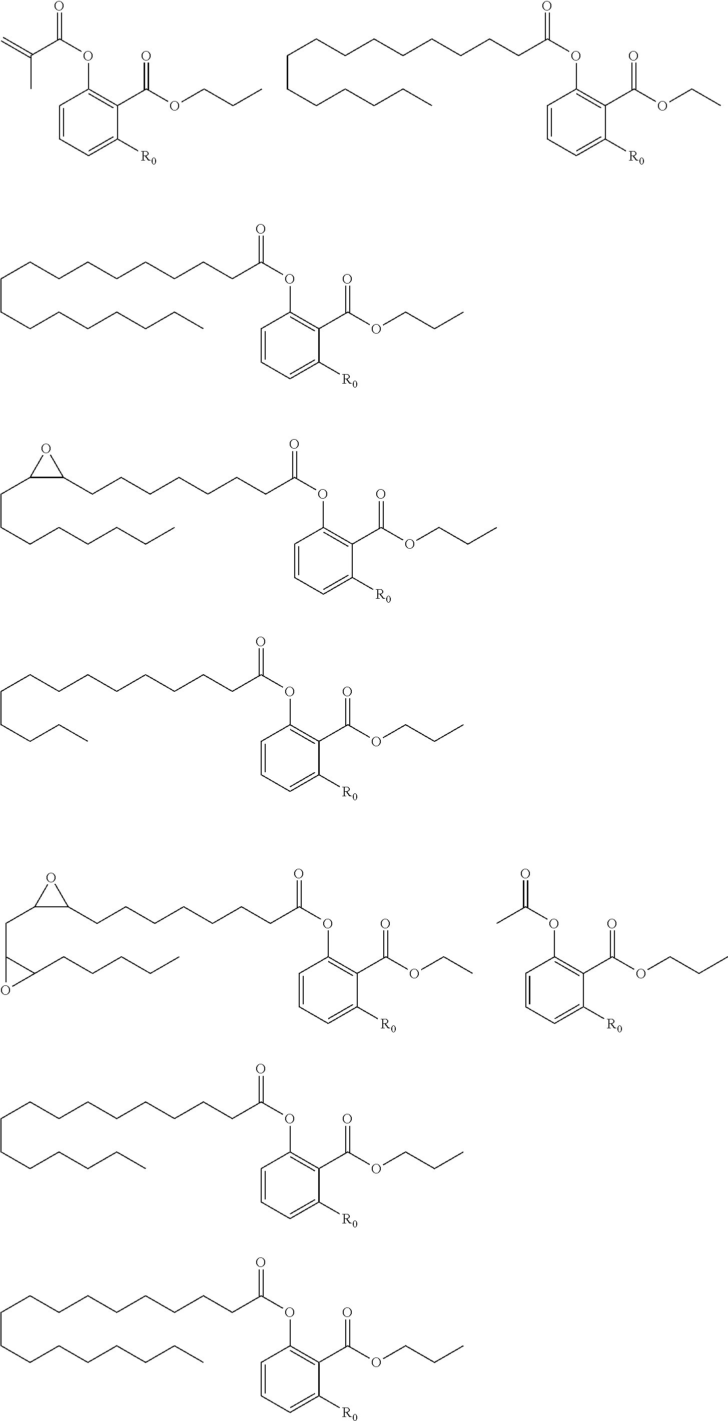

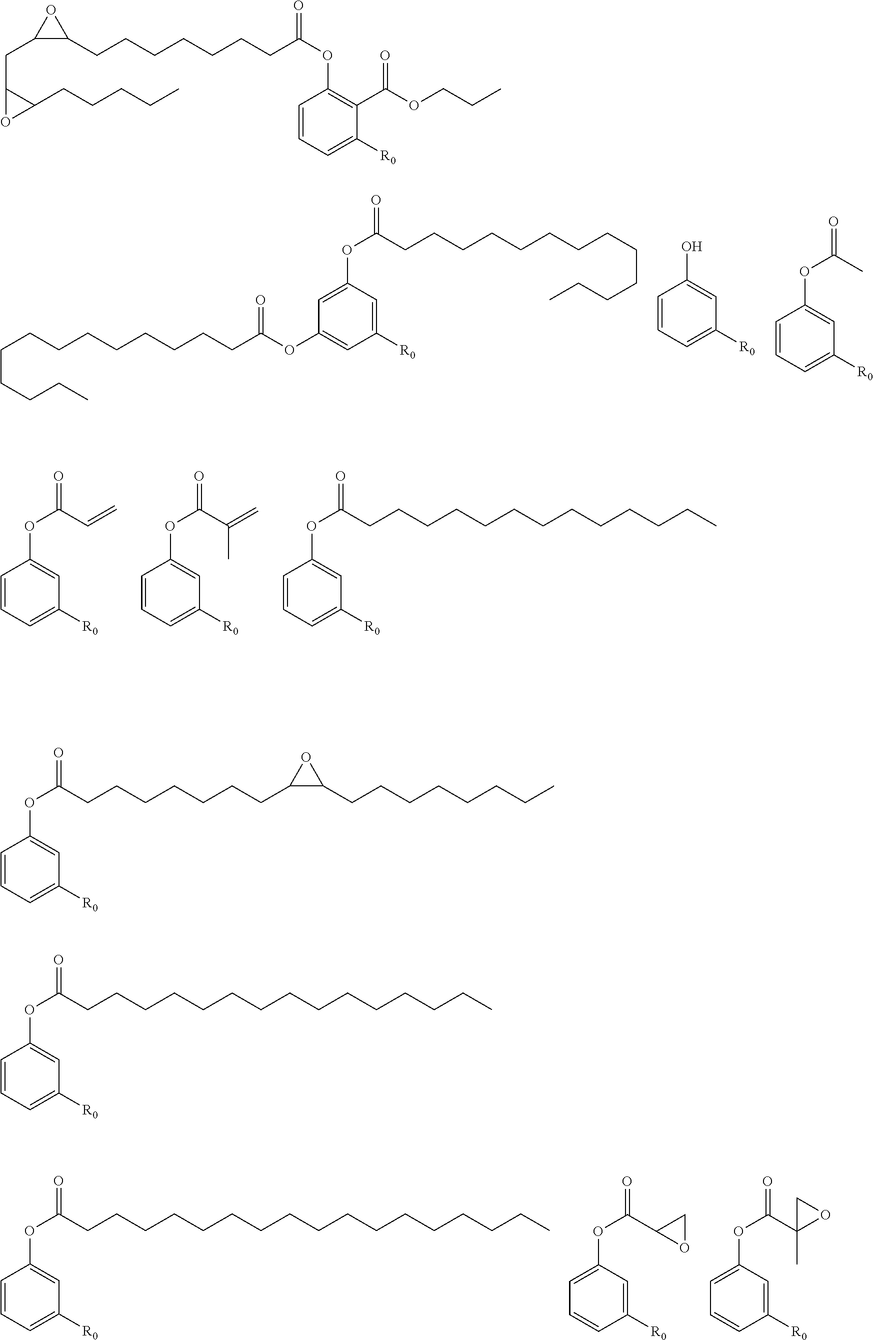

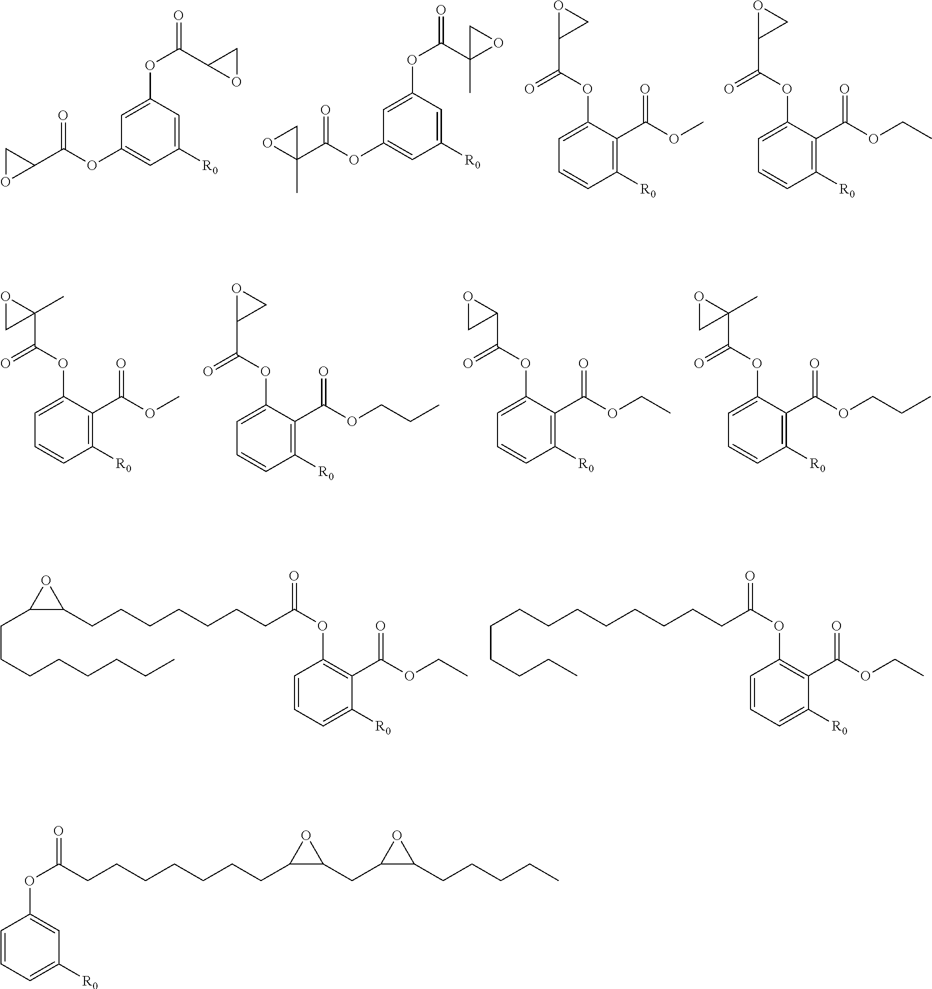

R₀ is selected from

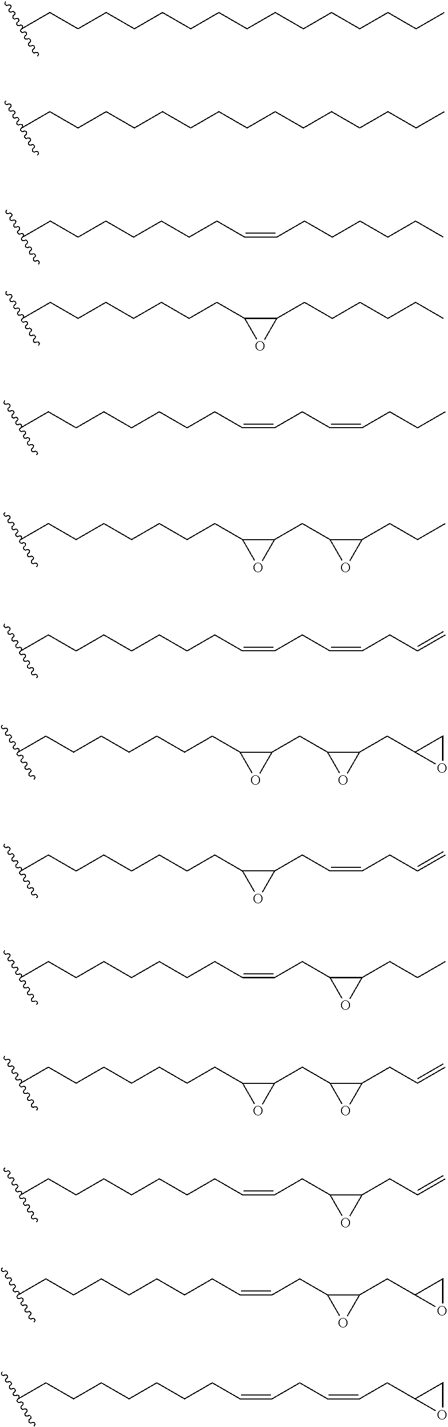

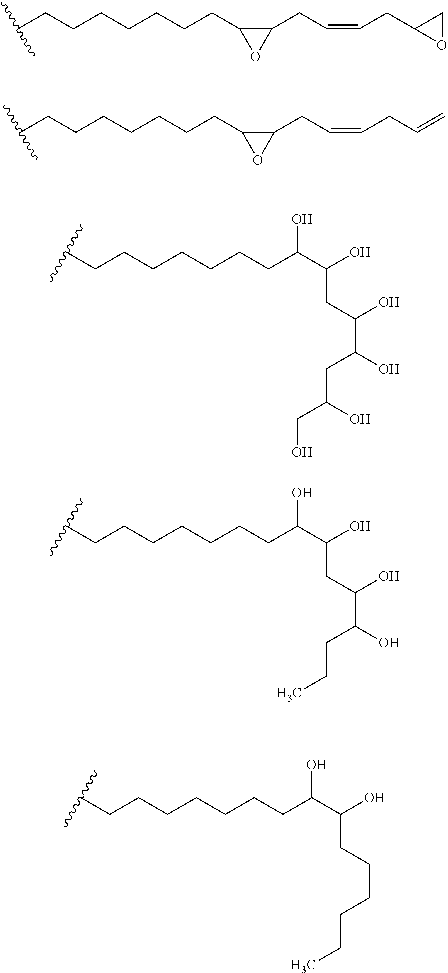

According to another particular embodiment, the present invention relates to the use as defined above, in which at least one compound of formula (2), (3), (4), (5), (6), (7), (8), (9), (10), (11), (12), (13) or (14) is derived from CNSL, in particular from undecarboxylated crude CNSL.

According to another particular embodiment, the present invention relates to the use as defined above, wherein said composition comprises at least two compounds of formulae (2), (3), (4), (5), (6), (7), (8), (9), (10), (11), (12), (13) or (14).

A second object of the present invention is a process for preparing a plastic material comprising or consisting of cellulose acetate, comprising a step of mixing cellulose acetate with a composition comprising at least one plasticizing agent for cellulose acetate, said composition comprising as plasticizing agent at least one compound derived from CNSL as defined above according to the invention or at least one compound of formula (1) as defined above, to obtain said plastic material.

Another object of the present invention is a process for preparing a plastic material comprising or consisting of cellulose acetate, comprising a step of mixing cellulose acetate with a composition comprising at least one primary plasticizer of cellulose acetate, said composition comprising as primary plasticizer at least one compound derived from CNSL as defined above according to the invention or at least one compound of formula (1) as defined above, to obtain said plastic material.

According to another particular embodiment, the present invention relates to a process for preparing a plastic material comprising or consisting of cellulose acetate, comprising a step of mixing cellulose acetate with a composition comprising at least one primary plasticizer of cellulose acetate, in an extruder, said composition comprising as primary plasticizer at least one compound derived from CNSL as defined above according to the invention or at least one compound of formula (1) as defined above, to obtain said plastic material.

Advantageously, the plastic material can be prepared without solvents.

According to another particular embodiment, the present invention relates to a process as defined above in which the concentration level by weight of the primary plasticizing agent is from 20 to 40%, preferably from 25 to 32%.

The 20 to 40% range comprises the ranges: from 20 to 22%; from 22 to 24%; from 24 to 26%; from 26 to 28%; from 28 to 30%; from 30 to 32%; from 32 to 34%; from 34 to 36%; from 36 to 38%; from 38 to 40%.

The 25 to 32 range comprises the ranges: from 25 to 26%; from 26 to 27%; from 27 to 28%; from 28 to 29%; from 29 to 30%; from 30 to 31%; from 31 to 32%.

According to another particular embodiment, the present invention relates to a process as defined above in which cellulose acetate and the primary plasticizing agent are mixed using an extruder.

Advantageously, the extruder is set to a temperature from 150 to 190° C. and a rotational speed from 150 to 200 rpm, preferably around 160 rpm.

As a non-limiting example, the extruder is a twin screw extruder (22), a Noztek Touch extruder.

According to another particular embodiment, the present invention relates to a process for preparing a plastic material comprising or consisting of cellulose acetate, comprising a step of mixing cellulose acetate with a composition comprising at least one primary plasticizing agent of cellulose acetate, in a solvent, to form a solution, said composition comprising as primary plasticizing agent at least one compound derived from CNSL as defined above according to the invention or at least one compound of formula (1) as defined above, to obtain said plastic material in the solvent.

According to another particular embodiment, the present invention relates to a process as defined above, in which cellulose acetate is chosen with a degree of substitution DS varying from 1.5 to 3.0, preferably from 2.0 to 2.8, in particular 2.5.

The “1.5 to 3.0” range includes the ranges: from 1.5 to 1.6; from 1.6 to 1.7; from 1.7 to 1.8; from 1.8 to 1.9; from 1.9 to 2.0; from 2.0 to 2.1; from 2.1 to 2.2; from 2.2 to 2.3; from 2.3 to 2.4; from 2.4 to 2.5; from 2.5 to 2.6; from 2.6 to 2.7; from 2.7 to 2.8; from 2.8 to 2.9; from 2.9 to 3.0.

According to another particular embodiment, the present invention relates to a process as defined above, in which the solvent used is selected from acetone, methanol, ethanol, ethyl acetate, thetrahydrofuran (THF), methyltetrahydrofuran (Me-THF), butanone, butyl acetate or chloroform, in particular acetone.

According to another particular embodiment, the present invention relates to a process as defined above, in which the mixing step is carried out at room temperature, from 20 to 30° C., at atmospheric pressure (1 atm i.e. 101,325 Pa).

The process for preparing the plastic material according to the invention requires few steps, and the mixing of cellulose acetate and the primary plasticizing agent can be carried out at room temperature, from 20 to 30° C. Advantageously, no heating step is required for incorporating the plasticizer.

According to another particular embodiment, the present invention relates to a process as defined above, further comprising a step of evaporating said solvent from said solution, to obtain said plastic material.

Advantageously, the solvent evaporation step is carried out at room temperature, from 20 to 30° C., at atmospheric pressure (1 atm or 101,325 Pa).

According to another particular embodiment, the present invention relates to a process as defined above, in which, the total amount by weight of cellulose acetate and primary plasticizer represents from 1 to 50% of the solution by weight, in particular from 10 to 30%, preferably 10%.

The 1 to 50% range comprises the ranges: from 1 to 5%; from 5 to 10%; from 10 to 15%; from 15 to 20%; from 20 to 25%; from 25 to 30%; from 30 to 35%; from 35 to 40%; from 40 to 45%; from 45 to 50%.

According to another particular embodiment, the present invention relates to a process as defined above, in which the ratio between the amount of primary plasticizing agent by weight and cellulose acetate by weight varies from 1 to 50% by weight, in particular from 10 to 30%, preferably about 30%.

According to another particular embodiment, the present invention relates to a process for preparing a plastic material comprising or consisting of cellulose acetate, comprising a step of mixing cellulose acetate with a composition comprising at least one primary plasticizing agent of cellulose acetate, in a solvent, to form a solution, said composition comprising as primary plasticizing agent at least one compound derived from CNSL according to the invention or at least one compound of formula (1) as defined above according to the invention, to obtain said plastic material in the solvent,

optionally further comprising a step of evaporating said solvent from said solution, to obtain said plastic material,
and/or wherein the total amount by weight of cellulose acetate and primary plasticizer is from 1 to 50% of the solution by weight, in particular from 10 to 30, preferably 10%;
and/or wherein the ratio between the amount of primary plasticizer by weight and cellulose acetate by weight ranges from 1 to 50% by weight, in particular from 10 to 30%, preferably about 30%.

The processes for preparing plastics by incorporating a plasticizing agent into a cellulose acetate-based matrix known to the skilled person in the prior art can be adapted for preparing a plastic material comprising or consisting of cellulose acetate, comprising as plasticizing agent at least one compound derived from CNSL as defined above according to the invention or at least one compound of formula (1) as defined above.

According to another particular embodiment, the present invention relates to a process as defined above, in which, in the composition comprising the primary plasticizer, the latter is a compound of formula (2)

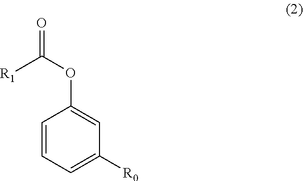

formula (2) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3, formula (2) in which R₁ represents:
   a linear alkyl group of 1 to 30 carbon atom(s), or
   a linear alkenyl group of 2 to 24 carbon atoms, comprising 1 to 6 alkenyl function(s), said alkenyl group being optionally substituted by a linear alkyl group of 1 to 5 carbon atom(s)
      R₁ optionally comprising one to six substituent(s), in particular one or two, selected from —OH, —OR₇ or OCOR₇, wherein R₇ is an alkyl group of 1 to 3 carbon atoms
      R₀ optionally comprising one to six substituent(s) selected from —OH, —OR₇ or OCOR₇,
      and further comprising a step A of preparing said compound of formula (2) from a compound of formula (15) with an acid of formula (16):

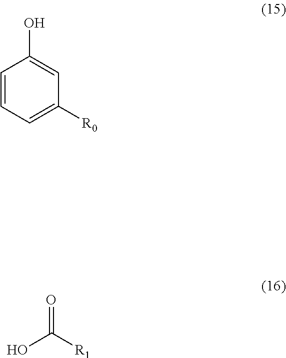

formula (15) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3, formula (16) in which R₁ represents:
a linear alkyl group of 1 to 30 carbon atom(s), or
a linear alkenyl group of 2 to 24 carbon atom(s), comprising 1 to 6 alkenyl function(s), said alkenyl group being optionally substituted by a linear alkyl group of 1 to 5 carbon atom(s),
   R₁ optionally comprising one to six substituent(s), in particular one or two, selected from —OH, —OR₇ or OCOR₇, wherein R₇ is an alkyl group of 1 to 3 carbon atoms
   R₀ optionally comprising one to six substituent(s) selected from —OH, —OR₇ or OCOR₇.

According to another particular embodiment, the present invention relates to a process as defined above, wherein, in the composition comprising the plasticizing agent, the latter is a compound of formula (2), and comprising the following steps:
a step A for the preparation of the compound (2) consisting of esterification of the compound of formula (15) with an acid of formula (16) to obtain the compound of formula (2),

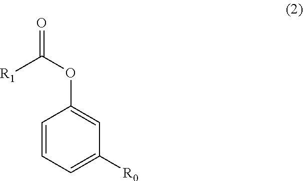

a step of mixing cellulose acetate with a composition comprising at least one plasticizing agent for cellulose acetate in a solvent to form a solution, said composition comprising as plasticizing agent said compound of formula (2), to obtain said plastic material in the solvent,
a step of evaporating said solvent from said solution to obtain said plastic material.

As a non-limiting example, step A of esterification of cardanol (1 equivalent) with an acid (1 equivalent) can be carried out in the presence of dimethylamino pyridine (DMAP) and two equivalents of 1-ethyl-3-(3-dimethylaminopropyl) carbodiimide (EDC), in which the mixture is stirred for 4 hours.

As a non-limiting example, step A of esterification of cardanol (1 equivalent) with an acid (1 equivalent) can be carried out in the presence of 1 equivalent of propylphosphonic anhydride (T3P) at 80° C. in ethyl acetate, the resulting mixture being stirred for approximately 4 hours.

As a non-limiting example, step A of esterification of cardanol (1 equivalent) with an acid (1 equivalent) can be carried out in the presence of triethylamine (Et3N) in dichloromethane (DCM) at 0° C., for approximately 18 hours.

According to another particular embodiment, the present invention relates to a process as defined above, in which, in the composition comprising the primary plasticizing agent, the latter is a compound of formula (3)

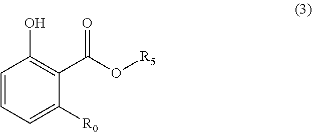

formula (3) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3, formula (3) in which R₅ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   R₅ optionally comprising one to six substituent(s), in particular one or two, selected from —OH, —OR₇ or OCOR₇, wherein R₇ is an alkyl group of 1 to 3 carbon atoms
   R₀ optionally comprising one to six substituent(s) selected from —OH, —OR₇ or OCOR₇,
   and further comprising a step B of preparing a compound of formula (3) from a compound of formula (17) and an alcohol of formula (18):

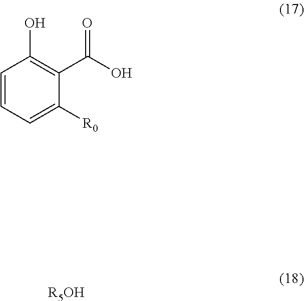

formula (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3, formula (18) in which R₅ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   R₅, in addition to the —OH group represented in formula (18), optionally comprising one to six substituent(s), in particular one or two, selected from —OH, —OR₇ or OCOR₇, wherein R₇ is an alkyl group of 1 to 3 carbon atoms
   R₀ optionally comprising one to six substituent(s) selected from —OH, —OR₇ or OCOR₇,

According to another particular embodiment, the present invention relates to a process as defined above, wherein, in the composition comprising the plasticizing agent, the latter is a compound of formula (3), and comprising the following steps:
a preparation step B of compound (3) consisting of esterification of compound of formula (17) with compound (18) to obtain a compound of formula (3),

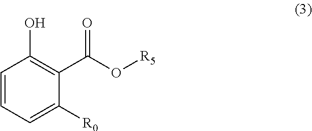

a step of mixing cellulose acetate with a composition comprising at least one plasticizing agent for cellulose acetate in a solvent to form a solution, said composition comprising as plasticizing agent said compound of formula (3), to obtain said plastic material in the solvent,
a step of evaporating said solvent from said solution to obtain said plastic material.

As a non-limiting example, the first step B of esterification of anacardic acid (1 equivalent) with alcohol (in excess) can be carried out in the presence of sulfuric acid in a mixture stirred and heated to reflux under argon for about 1 day.

According to another particular embodiment, the present invention relates to a process as defined above, in which, in the composition comprising the primary plasticizing agent, the latter is a compound of formula (4)

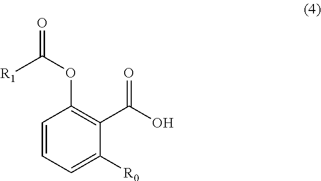

formula (4) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3.
   formula (4) in which R₁ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   R₁ optionally comprising one to six substituent(s), in particular one or two, selected from —OH, —OR₇ or OCOR₇, wherein R₇ is an alkyl group of 1 to 3 carbon atoms R₀ optionally comprising one to six substituent(s) selected from —OH, —OR₇ or OCOR₇, and further comprising a step C of preparing a compound of formula (4) from a compound of formula (17) and an acid of formula (19):

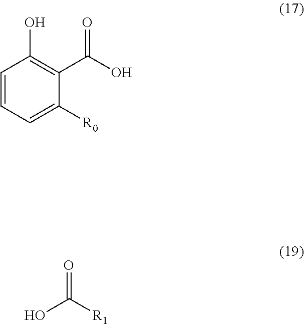

formula (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3, formula (19) in which R₁ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s), said group R₁ optionally comprising one to six substituent(s), in particular one or two, selected from —OH, —OR₇ or OCOR₇, wherein R₇ is an alkyl group of 1 to 3 carbon atoms said R₀ group optionally comprising one to six substituent(s) selected from —OH, —OR₇ or OCOR₇;

According to another particular embodiment, the present invention relates to a process as defined above, wherein, in the composition comprising the plasticizing agent, the latter is a compound of formula (4), and comprising the following steps:
a preparation step C of the compound of formula (4) consisting of an esterification step of the compound of formula (17) with the compound (19) to obtain the compound of formula (4),

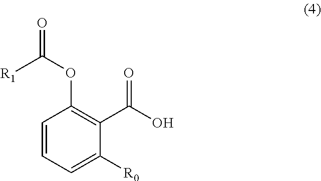

a step of mixing cellulose acetate with a composition comprising at least one plasticizing agent for cellulose acetate in a solvent to form a solution, said composition comprising as plasticizing agent said compound of formula (4), to obtain said plastic material in the solvent,
a step of evaporating said solvent from said solution to obtain said plastic material.

As a non-limiting example, step C of esterification of anacardic acid (1 equivalent) with an acid (1 equivalent) can be carried out in the presence of dimethylamino pyridine (DMAP) and two equivalents of 1-ethyl-3-(3-dimethylaminopropyl) carbodiimide (EDC), the mixture being stirred for approximately 4 hours.

As a non-limiting example, step C of esterification of anacardic acid (1 equivalent) with an acid (1 equivalent) can be carried out in the presence of 1 equivalent of propylphosphonic anhydride (T3P) in ethyl acetate at 80° C., for about 4 hours.

As a non-limiting example, step C of esterification of anacardic acid (1 equivalent) with an acid (1 equivalent) can be carried out in the presence of triethylamine (Et3N) in dichloromethane (DCM) at 0° C., for about 18 hours.

According to another particular embodiment, the present invention relates to a process as defined above, in which, in the composition comprising the primary plasticizing agent, the latter is a compound of formula (5)

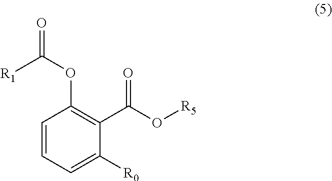

formula (5) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₁ and R₅ represent:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   R₁ and R₅ optionally comprising one to six substituent(s), in particular one or two, selected from —OH, —OR₇ or OCOR₇, wherein R₇ is an alkyl group of 1 to 3 carbon atoms
   R₀ optionally comprising one to six substituent(s) selected from —OH, —OR₇ or OCOR₇, and further comprising a step D of preparing a compound of formula (5) from a compound of formula (17) with an alcohol of formula (18) and an acid of formula (19):

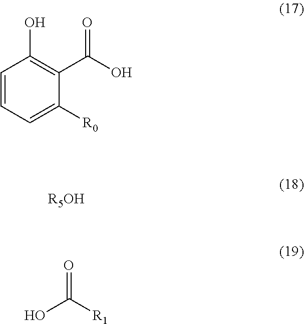

formula (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3, formulae (18) and (19) in which R₁ and R₅ independently represent:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s), said R₁ group optionally comprising one to six substituent(s), in particular one or two, selected from —OH, —OR₇ or OCOR₇, wherein R₇ is an alkyl group of 1 to 3 carbon atoms
   R₅, in addition to the —OH group represented in formula (18), optionally comprising one to six substituent(s), in particular one or two, selected from —OH, —OR₇ or OCOR₇
   said R group₀ optionally comprising one to six substituent(s) selected from —OH, —OR₇ or OCOR₇.

According to another particular embodiment, the present invention relates to a process as defined above, wherein, in the composition comprising the plasticizing agent, the latter is a compound of formula (5), and comprising the following steps:
a preparation step D of the compound of formula (5) consisting of:
   i) a first step of esterifying the compound of formula (17) with a compound (18) to obtain the intermediate compound:

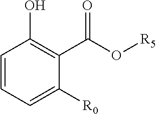

ii) a second esterification step with a compound of formula (19) to obtain the compound of formula (5),

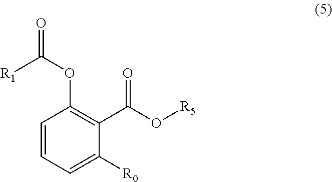

a step of mixing cellulose acetate with a composition comprising at least one plasticizing agent for cellulose acetate in a solvent to form a solution, said composition comprising as plasticizing agent said compound of formula (5), to obtain said plastic material in the solvent,
a step of evaporating said solvent from said solution to obtain said plastic material.

As a non-limiting example, the first step D of esterification i) of anacardic acid (1 equivalent) with alcohol (in excess) can be carried out in the presence of sulfuric acid in a mixture stirred and heated to reflux under argon for about 1 day.

As a non-limiting example, the second step D of esterification ii) with an acid (1 equivalent) can be carried out in the presence of dimethylamino pyridine (DMAP) and two equivalents of 1-ethyl-3-(3-dimethylaminopropyl) carbodiimide (EDC), the resulting mixture being stirred for 4 hours.

As a non-limiting example, the second step D of esterification ii) with an acid (1 equivalent) can be carried out in the presence of 1 equivalent of propylphosphonic anhydride (T3P) in ethyl acetate at 80° C., for about 4 hours.

As a non-limiting example, the second esterification step D ii) with an acid (1 equivalent) can be carried out in the presence of triethylamine (Et3N) in dichloromethane (DCM) at 0° C., for approximately 18 hours.

According to another particular embodiment, the present invention relates to a process as defined above, in which, in the composition comprising the primary plasticizing agent, the latter is a compound of formula (6)

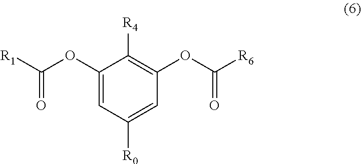

formula (6) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than
   R₄ represents:
a hydrogen atom,
a methyl group CH₃,
   R₁ and R₆ independently represent, in particular may be identical:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   R₁ and R₆ optionally comprising one to six substituent(s), in particular one or two, selected from —OH, —OR₇ or OCOR₇, wherein R₇ is an alkyl group of 1 to 3 carbon atoms
   R₀ optionally comprising one to six substituent(s) selected from —OH, —OR₇ or OCOR₇,
   and further comprising a step E of preparing said compound of formula (6) from a compound of formula (20) with an acid of formula (21) and an acid of formula (22)

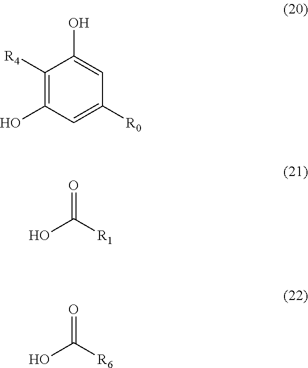

formula (20) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₄ represents:
a hydrogen atom,
a methyl group CH₃,
   formulae (21) and (22) in which R₁ and R₆ independently represent, in particular may be identical:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   R₁ and R₆ optionally comprising one to six substituent(s), in particular one or two, selected from —OH, —OR₇ or OCOR₇, wherein R₇ is an alkyl group of 1 to 3 carbon atoms
   R₀ optionally comprising one to six substituent(s) selected from —OH, —OR₇ or OCOR₇.

According to another particular embodiment, the present invention relates to a process as defined above, wherein, in the composition comprising the plasticizing agent, the latter is a compound of formula (6), and comprising the following steps:
a preparation step E of the compound of formula (6) consisting of esterification of the compound of formula (20) with an acid of formula (21) and an acid of formula (22), in particular the acids of formulae (21) and (22) being identical, to obtain the compound of formula (6),

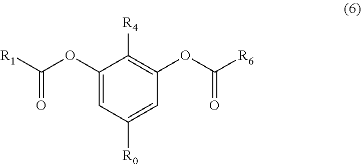

a step of mixing cellulose acetate with a composition comprising at least one plasticizing agent for cellulose acetate in a solvent to form a solution, said composition comprising as plasticizing agent said compound of formula (2), to obtain said plastic material in the solvent,
a step of evaporating said solvent from said solution to obtain said plastic material.

As a non-limiting example, the esterification step E of cardol (1 equivalent) with two acids (2 equivalents) can be carried out in the presence of two equivalents of propylphosphonic anhydride (T3P) in ethyl acetate at 80° C., for about 4 hours.

According to another particular embodiment, the present invention relates to a process as defined above, in which, in the composition of the primary plasticizer, the latter is a compound of formula (7)

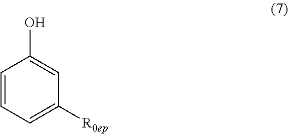

formula (7) in which R₀ₑₚ represents:
a linear alkyl group of 15 carbon atoms containing from 1 to 3 epoxide functions, or
a linear alkenyl group of 15 carbon atoms, comprising x alkenyl functions, x varying from 1 to 2, comprising y epoxide functions, y varying from 1 to 2, x+y being less than or equal to 3,
   R₀ₑₚ optionally comprising one to six substituent(s) selected from —OH, —OR₇ or OCOR₇, wherein R₇ is an alkyl group of 1 to 3 carbon atoms
   and further comprising a step F of preparing said compound of formula (7) from a compound of formula (15)

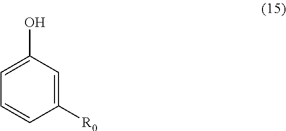

formula (15) in which R₀ represents:
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or OCOR₇, wherein R₇ is an alkyl group of 1 to 3 carbon atoms,

Thus said compound of formula (15) comprises at least one additional alkenyl function relative to the compound of formula (7), said function being epoxidized to form said compound of formula (7).

It is understood that the compound of formula (15) may be a compound already having epoxide functions, but that the latter comprises at least one additional alkenyl function compared with the compound of formula (7).

According to another particular embodiment, the present invention relates to a process as defined above, in which, in the composition comprising the plasticizing agent, the latter is a compound of formula (7), and comprising the following steps:
a preparation step F consisting of an epoxidation step of a compound of formula (15) to obtain the compound of formula (7):

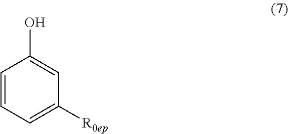

a step of mixing cellulose acetate with a composition comprising at least one plasticizing agent for cellulose acetate in a solvent to form a solution, said composition comprising as plasticizing agent said compound of formula (7), to obtain said plastic material in the solvent,
a step of evaporating said solvent from said solution to obtain said plastic material.

As a non-limiting example, the epoxidation step F of cardanol (1 equivalent) is carried out in the presence of formic acid (3 equivalents), sulfuric acid (0.01 equivalents) and hydrogen peroxide (2 equivalents) with stirring and heating at 65° C. for approximately 3 hours.

For example, according to the following reaction scheme:

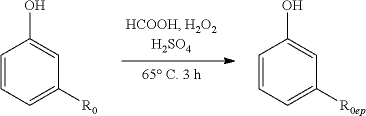

As a non-limiting example, the step F of epoxidation of cardanol (1 equivalent) is carried out in the presence of 70% metachloroperbenzoic acid (mCPBA) (2 equivalents) in dichloromethane (DCM) cold stirred (0° C.) and then stirred at room temperature for around 18 h.

According to another particular embodiment, the present invention relates to a process as defined above, in which, in the composition comprising the primary plasticizing agent, the latter is a compound of formula (8)

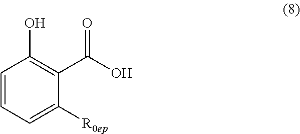

formula (8) in which R₀ₑₚ represents:
a linear alkyl group of 15 carbon atoms containing from 1 to 3 epoxide functions, or
a linear alkenyl group of 15 carbon atoms, comprising x alkenyl functions, x varying from 1 to 2, comprising y epoxide functions, y varying from 1 to 2, x+y being less than or equal to 3,
   R₀ₑₚ optionally comprising one to six substituent(s) selected from —OH, —OR₇ or OCOR₇, wherein R₇ is an alkyl group of 1 to 3 carbon atoms,
   and further comprising a step G of preparing a compound of formula (8) from a compound of formula (17)

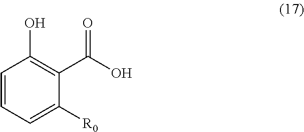

formula (17) in which R₀ represents:
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or OCOR₇, wherein R₇ is an alkyl group of 1 to 3 carbon atoms.

Thus said compound of formula (17) comprising at least one additional alkenyl function relative to the compound of formula (8), said function being epoxidized to form said compound of formula (8).

It is understood that the compound of formula (17) may be a compound already having epoxide functions, but that the latter comprises at least one additional alkenyl function compared with the compound of formula (8).

According to another particular embodiment, the present invention relates to a process as defined above, wherein, in the composition comprising the plasticizing agent, the latter is a compound of formula (8), and comprising the following steps:
a preparation step G of the compound of formula (8) consisting of an epoxidation step of the compound of formula (17) to obtain the compound of formula (8)

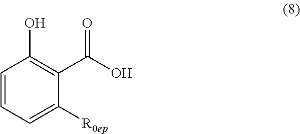

a step of mixing cellulose acetate with a composition comprising at least one plasticizing agent for cellulose acetate in a solvent to form a solution, said composition comprising as plasticizing agent said compound of formula (8), to obtain said plastic material in the solvent,
a step of evaporating said solvent from said solution to obtain said plastic material.

As a non-limiting example, the epoxidation step G of anacardic acid (1 equivalent) is carried out in the presence of formic acid (3 equivalents), sulfuric acid (0.01 equivalents) and hydrogen peroxide (2 equivalents) with stirring and heating at 65° C. for approximately 3 hours.

As a non-limiting example, the step G of epoxidation of anacardic acid (1 equivalent) is carried out in the presence of 70% metachloroperbenzoic acid (mCPBA) (2 equivalents) in dichloromethane (DCM) cold stirred (0° C.) and then stirred at room temperature for around 18 h.

According to another particular embodiment, the present invention relates to a process as defined above, in which, in the composition comprising the primary plasticizing agent, the latter is a compound of formula (9)

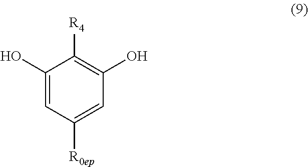

formula (9) in which R₀ₑₚ represents:
a linear alkyl group of 15 carbon atoms containing from 1 to 3 epoxide functions, or
a linear alkenyl group of 15 carbon atoms, comprising x alkenyl functions, x varying from 1 to 2, comprising y epoxide functions, y varying from 1 to 2, x+y being less than or equal to 3,
   R₀ₑₚ optionally comprising one to six substituent(s) selected from —OH, —OR₇ or OCOR₇, wherein R₇ is an alkyl group of 1 to 3 carbon atoms,
   R₄ represents:
a hydrogen atom,
a methyl group CH₃,
   and further comprising a step H of preparing said compound of formula (9) from a compound of formula (20)

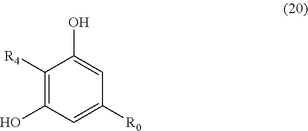

formula (20) in which R₀ represents:
a linear alkenyl group of 15 carbon atoms, comprising x alkenyl functions, x varying from 1 to 3, comprising y epoxide functions, y varying from 0 to 2, x+y being less than or equal to 3, R₀ optionally comprising one to six substituent(s) selected from —OH, —OR₇ or OCOR₇, wherein R₇ is an alkyl group of 1 to 3 carbon atoms
   R₄ represents:
a hydrogen atom,
a methyl group CH₃.

Thus the compound of formula (20) comprising at least one additional alkenyl function relative to the compound of formula (9), said function being epoxidized to form said compound of formula (9).

It is understood that the compound of formula (20) may be a compound already having epoxide functions, but that the latter comprises at least one additional alkenyl function compared with the compound of formula (9).

According to another particular embodiment, the present invention relates to a process as defined above, wherein in the composition comprising the plasticizing agent, the latter is a compound of formula (9), and comprising the following steps:
A preparation step H of the compound of formula (9) consisting of an epoxidation step of the compound of formula (20) to obtain the compound of formula (9),

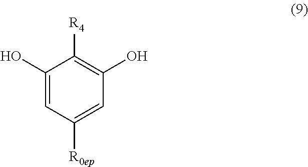

a step of mixing cellulose acetate with a composition comprising at least one plasticizing agent for cellulose acetate in a solvent to form a solution, said composition comprising as plasticizing agent said compound of formula (4), to obtain said plastic material in the solvent,
a step of evaporating said solvent from said solution to obtain said plastic material.

As a non-limiting example, the epoxidation step H of cardol (1 equivalent) is carried out in the presence of formic acid (3 equivalents), sulfuric acid (0.01 equivalents) and hydrogen peroxide (2 equivalents) with stirring and heating at 65° C. for approximately 3 hours.

As a non-limiting example, the epoxidation step H of the cardol (1 equivalent) is carried out in the presence of 70% metachloroperbenzoic acid (mCPBA) (2 equivalents) in dichloromethane (DCM), cold stirred (0° C.) and then stirred at room temperature for around 18 h.

According to another particular embodiment, the present invention relates to a process as defined above, in which, in the composition comprising the primary plasticizing agent, the latter is a compound of formula (10)

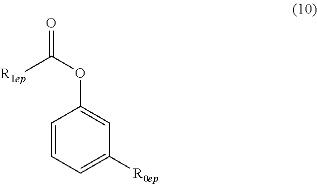

formula (10) in which R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ₑₚ optionally comprising one to six substituent(s) selected from —OH, —OR₇ or OCOR₇, wherein R₇ is an alkyl group of 1 to 3 carbon atoms,
   R₁ₑₚ represents:
a linear alkyl group of 1 to 30 carbon atom(s), optionally containing 1 to 6 epoxide function(s), or
a linear alkenyl group of 2 to 24 carbon atoms, optionally comprising 1 to 6 epoxide function(s), said alkenyl group being optionally substituted by an alkyl group of 1 to 5 carbon atom(s),
   R₁ₑₚ optionally comprising one or six substituents, in particular one or two, selected from —OH, —OR₇ or OCOR₇, wherein R₇ is an alkyl group of 1 to 3 carbon atoms,
   with the proviso that at least one of the R₀ₑₚ or R₁ₑₚ groups contains at least one epoxide function,
   and further comprising a step A of preparing a compound of formula (2) from a compound of formula (15) with an acid of formula (16):

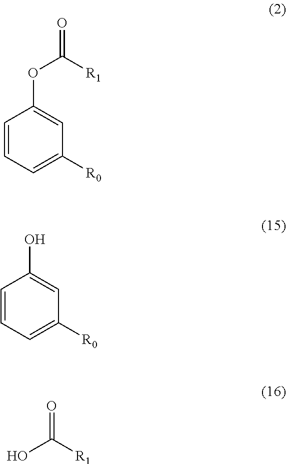

formulae (2) and (15) in which R₀ represents:
a linear alkyl group of 15 carbon atoms optionally containing from 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or OCOR₇, wherein R₇ is an alkyl group of 1 to 3 carbon atoms,
   formulae (2) and (16) in which R₁ represents:
a linear alkyl group of 1 to 30 carbon atoms, or
a linear alkenyl group of 2 to 24 carbon atoms, comprising from 1 to 6 alkenyl functions, said alkenyl group being optionally substituted by a linear alkyl group of 1 to 5 carbon atoms,
   R₁ optionally comprising one to six substituents, in particular one or two, selected from —OH, —OR₇ or OCOR₇, wherein R₇ is an alkyl group of 1 to 3 carbon atoms,
   and further comprising a step I of preparing the compound of formula (10) from a compound of formula (2),
   with the proviso that at least one of the R₀ or R₁ groups contains an alkenyl function,

Thus the compound of formula (2) comprises at least one additional alkenyl function relative to the compound of formula (10), said function being epoxidized to form said compound of formula (10).

It is understood that the compound of formula (2) may be a compound which already has epoxide functions, but that the latter comprises at least one additional alkenyl function compared with the compound of formula (10).

The additional epoxide function of the compound of formula (10) is located in the same position as the additional alkenyl function of compound (3). In simpler terms, the compound of formula (10) is derived from an epoxidation of an alkenyl function present in the compound of formula (3).

This embodiment comprises a step A of esterification of the cardanol of formula (15) with an acid of formula (16) followed by epoxidation of at least one alkenyl function in the compound of formula (2).

Advantageously, epoxidations of several alkenyl functions present in compound (2) can be implemented to obtain compound (10). These epoxidations can be successive or simultaneous.

It is also possible to isolate compounds with different degrees of epoxidation by methods known to those skilled in the art.

According to another particular embodiment, the present invention relates to a process as defined above, in which, in the composition comprising the plasticizing agent, the latter is a compound of formula (10), and

comprising the following steps:
a preparation step A of the compound of formula (2) consisting of an esterification step of the compound of formula (15) with an acid of formula (16) to obtain the compound of formula (2),

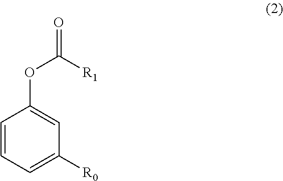

a preparation step I of the compound of formula (10) which consists of an epoxidation step of the compound of formula (2) to obtain the compound of formula (10),

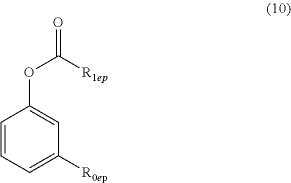

a step of mixing cellulose acetate with a composition comprising at least one plasticizing agent for cellulose acetate in a solvent to form a solution, said composition comprising as plasticizing agent said compound of formula (10), to obtain said plastic material in the solvent,
a step of evaporating said solvent from said solution to obtain said plastic material.

As a non-limiting example, the esterification step A of cardanol (1 equivalent) with an acid (1 equivalent) can be carried out in the presence of dimethylamino pyridine (DMAP) and two equivalents of 1-ethyl-3-(3-dimethylaminopropyl) carbodiimide (EDC), the resulting mixture being stirred for 4 hours.

As a non-limiting example, the esterification step A of cardanol (1 equivalent) with an acid (1 equivalent) can be carried out in the presence of one equivalent of propylphosphonic anhydride (T3P) at 80° C. in ethyl acetate, for approximately 4 hours.

As a non-limiting example, the esterification step A of cardanol (1 equivalent) with an acid (1 equivalent) can be carried out in the presence of triethylamine (Et3N) in dichloromethane (DCM) at 0° C., for approximately 18 hours.

By way of a non-limiting example, the epoxidation step I epoxidation of compound (2) (1 equivalent) is carried out in the presence of formic acid (3 equivalents), sulfuric acid (0.01 equivalents) and hydrogen peroxide (2 equivalents) with stirring and heating at 65° C. for approximately 3 hours.

By way of a non-limiting example, the epoxidation step I of compound (2) (1 equivalent) is carried out in the presence of 70% metachloroperbenzoic acid (mCPBA) (2 equivalents) in dichloromethane (DCM), cold stirred (0° C.) and then stirred at room temperature for around 18 h.

For example, according to the following reaction scheme:

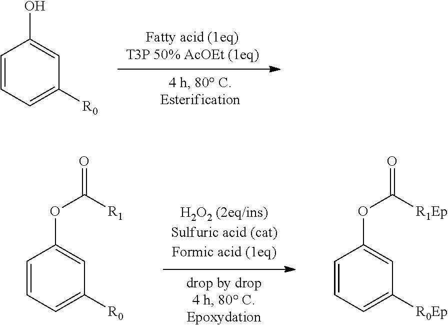

According to another particular embodiment, the present invention relates to a process as defined above, wherein, in the composition comprising the primary plasticizing agent, the latter is a compound of formula (11),

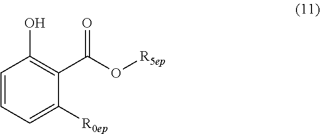

formula (11) in which R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ₑₚ optionally comprising one to six substituent(s) selected from —OH, —OR₇ or OCOR₇, wherein R₇ is an alkyl group of 1 to 3 carbon atoms
   R₅ₑₚ represents:
a linear alkyl group of 1 to 30 carbon atoms, optionally containing 1 to 6 epoxide functions, or
a linear alkenyl group of 2 to 24 carbon atoms, optionally comprising 1 to 6 epoxide function(s), said alkenyl group being optionally substituted by an alkyl group of 1 to 5 carbon atom(s),
   R₅ₑₚ optionally comprising one to six, in particular one or two, substituent(s) selected from —OH, —OR₇ or OCOR₇, wherein R₇ is an alkyl group of 1 to 3 carbon atoms,
   with the proviso that at least one of the R₀ₑₚ or R₅ₑₚ groups contains at least one epoxide function,
   and further comprising a preparation step B of a compound of formula (3) from a compound of formula (17) and an alcohol of formula (18):

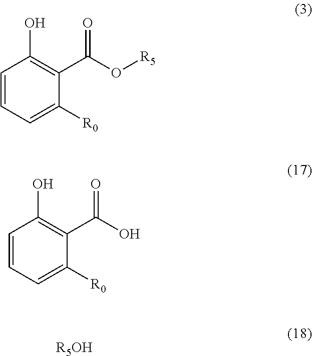

formulae (3) and (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or OCOR₇, wherein R₇ is an alkyl group of 1 to 3 carbon atoms,
   formulae (3) and (18) in which R₅ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   said R group₅, in addition to the —OH group represented in formula (18), optionally comprising one to six, in particular one or two, substituent(s) selected from —OH, —OR₇ and OCOR₇, wherein R₇ is an alkyl group of 1 to 3 carbon atom(s),
   and further comprising a step of preparing J a compound of formula (11) from a compound of formula (3),
   with the proviso that at least one of the R₀ or R₅ groups contains an alkenyl function,

Thus the compound of formula (3) comprises at least one additional alkenyl function relative to the compound of formula (11), said function being epoxidized to form said compound of formula (11).

It is understood that the compound of formula (3) may be a compound already having epoxide functions, but that the latter comprises at least one additional alkenyl function compared with the compound of formula (11).

The additional epoxide function of the compound of formula (11) is located in the same position as the additional alkenyl function of compound (3). In simpler terms, the compound of formula (11) results from epoxidation of an alkenyl function present in the compound of formula (3).

This embodiment comprises a step B of esterification of anacardic acid of formula (17) with an alcohol of formula (18) to obtain the ester of formula (3), followed by epoxidation of at least one alkenyl function of the compound of formula (3).

Advantageously, epoxidations of several alkenyl functions present in compound (3) can be implemented to obtain compound (11). These epoxidations can be successive or simultaneous.

It is also possible to isolate compounds with different degrees of epoxidation by methods known to those skilled in the art.

According to another particular embodiment, the present invention relates to a process as defined above, wherein, in the composition comprising the plasticizing agent, the latter is a compound of formula (11), and comprising the following steps:
a preparation step B of the compound of formula (3) consisting of esterification B of the compound of formula (17) with a compound (18) to obtain the compound of formula (3),

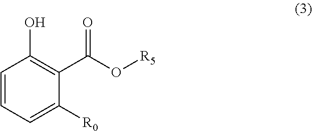

a preparation step J of the compound of formula (11) consisting of epoxidation of the compound of formula (3) to obtain the compound of formula (11)

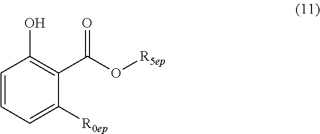

a step of mixing cellulose acetate with a composition comprising at least one plasticizing agent for cellulose acetate in a solvent to form a solution, said composition comprising as plasticizing agent said compound of formula (11), to obtain said plastic material in the solvent,
a step of evaporating said solvent from said solution to obtain said plastic material.

As a non-limiting example, the first B of esterification of anacardic acid (1 equivalent) with alcohol (in excess) can be carried out in the presence of sulfuric acid in a mixture stirred and heated to reflux under argon for about 1 day.

As a non-limiting example, epoxidation step J of compound (3) (1 equivalent) is carried out in the presence of formic acid (3 equivalents), sulfuric acid (0.01 equivalents) and hydrogen peroxide (2 equivalents) with stirring and heating at 65° C. for approximately 3 hours.

As a non-limiting example, epoxidation step J of compound (3) (1 equivalent) is carried out in the presence of 70% metachloroperbenzoic acid (mCPBA) (2 equivalents) in dichloromethane (DCM), cold stirred (0° C.) and then stirred at room temperature for around 18 h.

According to another particular embodiment, the present invention relates to a process as defined above, in which, in the composition comprising the primary plasticizing agent, the latter is a compound of formula (12)

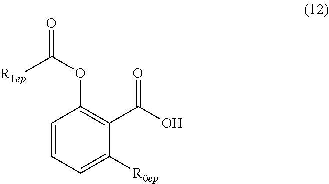

formula (12) in which R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ₑₚ optionally comprising one to six substituent(s) selected from —OH, —OR₇ or OCOR₇, wherein R₇ is an alkyl group of 1 to 3 carbon atoms,
   R₁ₑₚ represents:
a linear alkyl group of 1 to 30 carbon atom(s), optionally containing 1 to 6 epoxide function(s), or
a linear alkenyl group of 2 to 24 carbon atoms, optionally comprising 1 to 6 epoxide function(s), said alkenyl group being optionally substituted by an alkyl group of 1 to 5 carbon atom(s),
   R₁ₑₚ optionally comprising one to six substituents, in particular one or two, selected from —OH, —OR₇ or OCOR₇, wherein R₇ is an alkyl group of 1 to 3 carbon atoms,
   with the proviso that at least one of the R₀ₑₚ or R₁ₑₚ groups contains at least one epoxide function,
   and further comprising a step C of preparing a compound of formula (4) from a compound of formula (17) and an acid of formula (19):

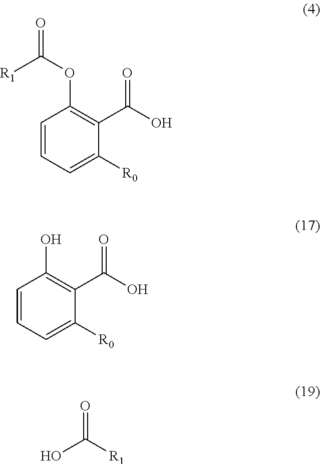

formulae (4) and (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or OCOR₇, wherein R₇ is an alkyl group of 1 to 3 carbon atoms,
   formulae (4) and (19) in which R₁ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   said R₁ group optionally comprising one to six substituent(s), in particular one or two, selected from —OH, —OR₇ and OCOR₇, wherein R₇ is an alkyl group of 1 to 3 carbon atom(s),
   with the proviso that at least one of the R₀ or R₁ groups contains an alkenyl function,
   and further comprising a step K of preparing a compound of formula (12) from a compound of formula (4).

Thus the compound of formula (4) comprises at least one additional alkenyl function relative to the compound of formula (12), said function being epoxidized to form said compound of formula (12).

It is understood that the compound of formula (4) may be a compound already having epoxide functions, but that the latter comprises at least one additional alkenyl function compared with the compound of formula (12).

The additional epoxide function of the compound of formula (12) is located in the same position as the additional alkenyl function of compound (4). In simpler terms, the compound of formula (12) is derived from an epoxidation of an alkenyl function present in the compound of formula (4).

This embodiment comprises a step C of esterification on the phenol function of anacardic acid of formula (17) with a carboxylic acid of formula (19) to obtain the ester of formula (4) followed by epoxidation of at least one alkenyl function of the compound of formula (4).

Advantageously, epoxidations of several alkenyl functions present in compound (4) can be implemented to obtain compound (12). These epoxidations can be successive or simultaneous.

It is also possible to isolate compounds with different degrees of epoxidation by methods known to those skilled in the art.

According to another particular embodiment, the present invention relates to a process as defined above, wherein, in the composition comprising the plasticizing agent, the latter is a compound of formula (12), and comprising the following steps:
a preparation step C of the compound of formula (4) consisting of an esterification step of the compound of formula (17) with a compound (19) to obtain the compound of formula (4),

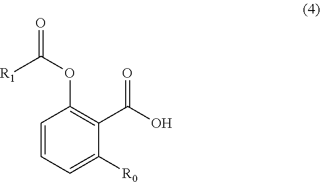

a preparation step K for preparing the compound of formula (12) consisting of a step of epoxidizing the compound of formula (4) to obtain the compound of formula (12),

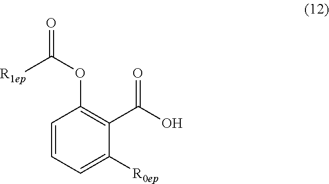

a step of mixing cellulose acetate with a composition comprising at least one plasticizing agent for cellulose acetate in a solvent to form a solution, said composition comprising as plasticizing agent said compound of formula (12), to obtain said plastic material in the solvent,
a step of evaporating said solvent from said solution to obtain said plastic material.

As a non-limiting example, the esterification step C of anacardic acid (17) (1 equivalent) with an acid (1 equivalent) can be carried out in the presence of dimethylamino pyridine (DMAP) and two equivalents of 1-ethyl-3-(3-dimethylaminopropyl) carbodiimide (EDC), the resulting mixture being stirred for 4 hours.

As a non-limiting example, the esterification step C of anacardic acid (17) (1 equivalent) with a carboxylic acid (1 equivalent) can be carried out in the presence of one equivalent of propylphosphonic anhydride (T3P) at 80° C. in ethyl acetate, for about 4 hours.

As a non-limiting example, the esterification step C of anacardic acid (17) (1 equivalent) with an acid (1 equivalent) can be carried out in the presence of triethylamine (Et3N) in dichloromethane (DCM) at 0° C., for about 18 hours.

As a non-limiting example, epoxidation step K of compound (4) (1 equivalent) is carried out in the presence of formic acid (3 equivalents), sulfuric acid (0.01 equivalents) and hydrogen peroxide (2 equivalents) with stirring and heating at 65° C. for approximately 3 hours.

As a non-limiting example, epoxidation step K of compound (4) (1 equivalent) is carried out in the presence of 70% metachloroperbenzoic acid (mCPBA) (2 equivalents) in dichloromethane (DCM), cold stirred (0° C.) and then stirred at room temperature for around 18 h.

According to another particular embodiment, the present invention relates to a process as defined above, in which, in the composition comprising the primary plasticizer, the latter is a compound of formula (13)

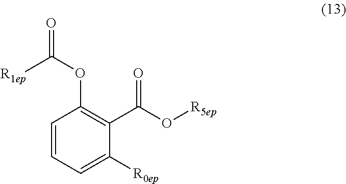

formula (13) in which R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ₑₚ optionally comprising one to six substituent(s) selected from —OH, —OR₇ or OCOR₇, wherein R₇ is an alkyl group of 1 to 3 carbon atoms,
   R₁ₑₚ and R₅ₑₚ independently represent:
a linear alkyl group of 1 to 30 carbon atom(s), optionally containing 1 to 6 epoxide function(s), or
a linear alkenyl group of 2 to 24 carbon atoms, optionally comprising 1 to 6 epoxide function(s), said alkenyl group being optionally substituted by an alkyl group of 1 to 5 carbon atom(s),
   R₁ₑₚ and R₅ₑₚ optionally comprising one to six substituent(s), in particular one or two, selected from —OH, —OR₇ or OCOR₇, wherein R₇ is an alkyl group of 1 to 3 carbon atoms,
   with the proviso that at least one of the R₀ₑₚ, R₁ₑₚ or R₅ₑₚ groups contains at least one epoxide function,
   and further comprising a of preparing step D a compound of formula (5) from a compound of formula (17) with an alcohol of formula (18) and an acid of formula (19),

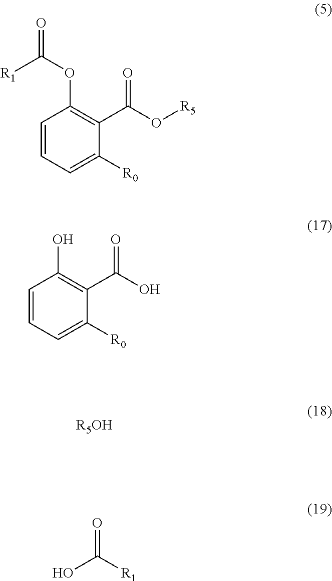

formulae (5) and (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or OCOR₇, wherein R₇ is an alkyl group of 1 to 3 carbon atoms,
   formulae (5), (18) and (19) in which R₁ and R₅ independently represent:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s), said group R₁, optionally comprising one to six substituent(s), in particular one or two, selected from —OH, —OR₇ and OCOR₇, wherein R₇ is an alkyl group of 1 to 3 carbon atom(s), said R₅ group, in addition to the —OH group represented in formula (18), optionally comprising one to six substituent(s), in particular one or two, selected from —OH, —OR₇ and OCOR₇,
   with the proviso that at least one of the R₀, R₁ or R₅ groups contains an alkenyl function and further comprising a step L of preparing a compound of formula (13) from a compound of formula (5).

Thus the compound of formula (5) comprises at least one additional alkenyl function relative to the compound of formula (13), said function being epoxidized to form said compound of formula (13).

It is understood that the compound of formula (5) may be a compound already having epoxide functions, but that the latter comprises at least one additional alkenyl function compared with the compound of formula (13).

The additional epoxide function of the compound of formula (13) is located in the same position as the additional alkenyl function of compound (5). In simpler terms, the compound of formula (13) is derived from an epoxidation of an alkenyl function present in the compound of formula (5).

This embodiment comprises a step D of esterification on the acid function and on the phenol function of anacardic acid of formula (17) with an alcohol of formula (18) and a carboxylic acid of formula (19) to obtain the compound of formula (5) followed by epoxidation of at least one alkenyl function of the compound of formula (5).

Advantageously, epoxidations of several alkenyl functions present in compound (5) can be implemented to obtain compound (13). These epoxidations can be successive or simultaneous.

It is also possible to isolate compounds with different degrees of epoxidation by methods known to those skilled in the art.

According to another particular embodiment, the present invention relates to a process as defined above, wherein, in the composition comprising the plasticizing agent, the latter is a compound of formula (13), and comprising the following steps:
a preparation step D of the compound (5) consisting of:
   i) a first step of esterifying the compound of formula (17) with a compound (18) to obtain an intermediate compound of formula (3),

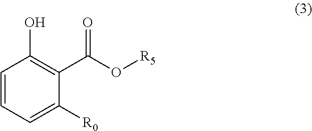

ii) a second esterification step with a compound of formula (19) to obtain the compound of formula (5),

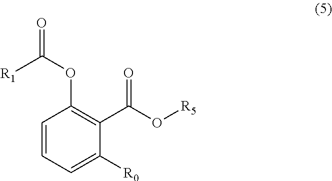

a preparation step L of the compound of formula (13) consisting of an epoxidation step of the compound of formula (5) to obtain the compound of formula (13)

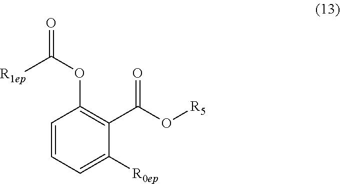

a step of mixing cellulose acetate with a composition comprising at least one plasticizing agent for cellulose acetate in a solvent to form a solution, said composition comprising as plasticizing agent said compound of formula (13), to obtain said plastic material in the solvent,
a step of evaporating said solvent from said solution to obtain said plastic material.

As a non-limiting example, the first step D of esterification i) of anacardic acid (1 equivalent) with alcohol (in excess) can be carried out in the presence of sulfuric acid in a mixture stirred and heated to reflux under argon for about 1 day.

As a non-limiting example, the second step D of esterification ii) with an acid (1 equivalent) can be carried out in the presence of dimethylamino pyridine (DMAP) and two equivalents of 1-ethyl-3-(3-dimethylaminopropyl) carbodiimide (EDC), the resulting mixture being stirred for 4 hours.

As a non-limiting example, the second step D of esterification ii) with an acid (1 equivalent) can be carried out in the presence of 1 equivalent of propylphosphonic anhydride (T3P) in ethyl acetate at 80° C., for about 4 hours.

As a non-limiting example, the second esterification step D ii) with an acid (1 equivalent) can be carried out in the presence of triethylamine (Et3N) in dichloromethane (DCM) at 0° C., for approximately 18 hours.

As a non-limiting example, epoxidation step L of compound (5) (1 equivalent) is carried out in the presence of formic acid (3 equivalents), sulfuric acid (0.01 equivalents) and hydrogen peroxide (2 equivalents) with stirring and heating at 65° C. for approximately 3 hours.

As a non-limiting example, epoxidation step L of compound (5) (1 equivalent) is carried out in the presence of 70% metachloroperbenzoic acid (mCPBA) (2 equivalents) in dichloromethane (DCM), cold stirred (0° C.) and then stirred at room temperature for around 18 h.

For example, according to the following 3-step reaction scheme:

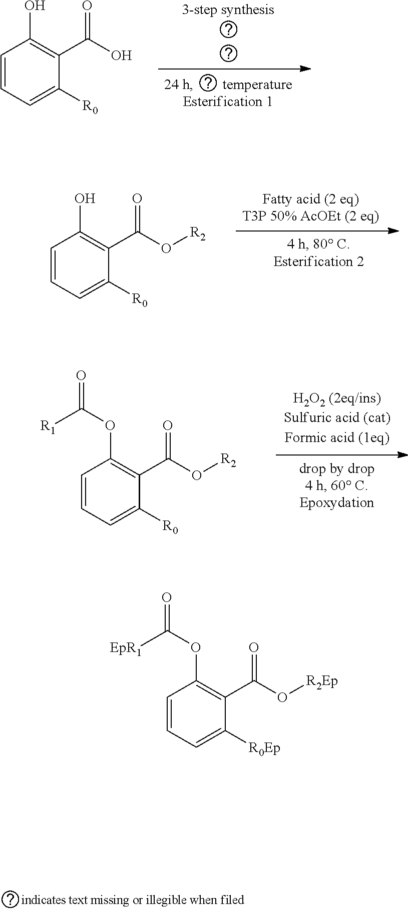

According to another particular embodiment, the present invention relates to a process as defined above, in which, in the composition comprising the primary plasticizing agent, the latter is a compound of formula (14)

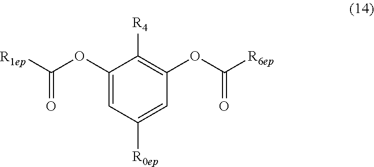

formula (14) in which R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ₑₚ optionally comprising one to six substituent(s) selected from —OH, —OR₇ or OCOR₇, wherein R₇ is an alkyl group of 1 to 3 carbon atoms,
   R₄ is selected from:
a hydrogen atom,
a methyl CH₃,
   R₁ₑₚ and R₆ₑₚ independently represent, in particular may be identical:
a linear alkyl group of 1 to 30 carbon atoms, optionally containing 1 to 6 epoxide functions, or
a linear alkenyl group of 2 to 24 carbon atoms, optionally comprising 1 to 6 epoxide function(s), said alkenyl group being optionally substituted by an alkyl group of 1 to 5 carbon atom(s),
   R₁ₑₚ and R₆ₑₚ optionally comprising one to six substituent(s), in particular one or two, selected from —OH, —OR₇ or OCOR₇, wherein R₇ is an alkyl group of 1 to 3 carbon atoms,
   with the proviso that at least one of the R₀ₑₚ, R₁ₑₚ or R₆ₑₚ groups contains at least one epoxide function,
   and further comprising a step E of preparing the compound of formula (6) from a compound of formula (20) with an acid of formula (21) and an acid of formula (22):

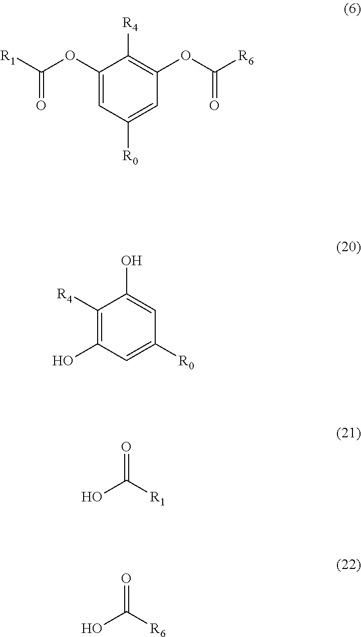

formulae (6) and (20) in which R₀ represent:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or OCOR₇, wherein R₇ is an alkyl group of 1 to 3 carbon atoms,
   R₄ represents:
a hydrogen atom,
a methyl group CH₃
   formulae (6), (21) and (22) in which R₁ and R₆ independently represent:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s), said R group₄ optionally comprising one to six substituent(s), in particular one or two, selected from —OH, —OR₇ and OCOR₇, wherein R₇ is an alkyl group of 1 to 3 carbon atom(s),
   with the proviso that at least one of the R₀, R₁ or R₆ groups contains an alkenyl function
   and further comprising a step M of preparing the compound of formula (14) from a compound of formula (6).

Thus the compound of formula (6) comprises at least one additional alkenyl function relative to the compound of formula (14), said function being epoxidized to form said compound of formula (14).

It is understood that the compound of formula (6) may be a compound already having epoxide functions, but that the latter comprises at least one additional alkenyl function compared with the compound of formula (14).

The additional epoxide function of the compound of formula (14) is located in the same position as the additional alkenyl function of compound (6). In simpler terms, the compound of formula (14) results from epoxidation of an alkenyl function present in the compound of formula (6).

This method comprises a step E of esterification of the two alcohol functions of the compound of formula (20) with two fatty acids of formulae (21) and (22) to obtain the diester of formula (6), followed by epoxidation of at least one alkenyl function of the compound of formula (6).

Advantageously, epoxidations of several alkenyl functions present in compound (6) can be implemented to obtain compound (14). These epoxidations can be successive or simultaneous.

It is also possible to isolate compounds with different degrees of epoxidation by methods known to those skilled in the art.

According to another particular embodiment, the present invention relates to a process as defined above, wherein, in the composition comprising the plasticizing agent, the latter is a compound of formula (14), and comprising the following steps:
a preparation step E of the compound of formula (6) consisting of esterification of the compound of formula (20) with an acid of formula (21) and an acid of formula (22), in particular the acids of formulae (21) and (22) are identical, to obtain the compound of formula (6),

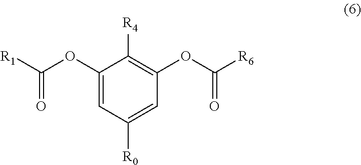

a preparation step M of the compound of formula (14) consisting of epoxidation of the compound of formula (6) to obtain the compound of formula (14),

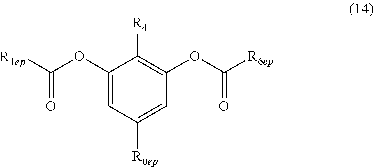

a step of mixing cellulose acetate with a composition comprising at least one plasticizing agent for cellulose acetate in a solvent to form a solution, said composition comprising as plasticizing agent said compound of formula (14), to obtain said plastic material in the solvent,
a step of evaporating said solvent from said solution to obtain said plastic material.

As a non-limiting example, the esterification step E of cardol (1 equivalent) with two acids (2 equivalents) can be carried out in the presence of two equivalents of propylphosphonic anhydride (T3P) in ethyl acetate at 80° C., for about 4 hours.

As a non-limiting example, epoxidation step M of compound (6) (1 equivalent) is carried out in the presence of formic acid (3 equivalents), sulfuric acid (0.01 equivalents) and hydrogen peroxide (2 equivalents) with stirring and heating at 65° C. for approximately 3 hours.

As a non-limiting example, epoxidation step M of compound (6) (1 equivalent) is carried out in the presence of 70% metachloroperbenzoic acid (mCPBA) (2 equivalents) in dichloromethane (DCM), cold stirred (0° C.) and then stirred at room temperature for around 18 h.

For example, according to the following diagram:

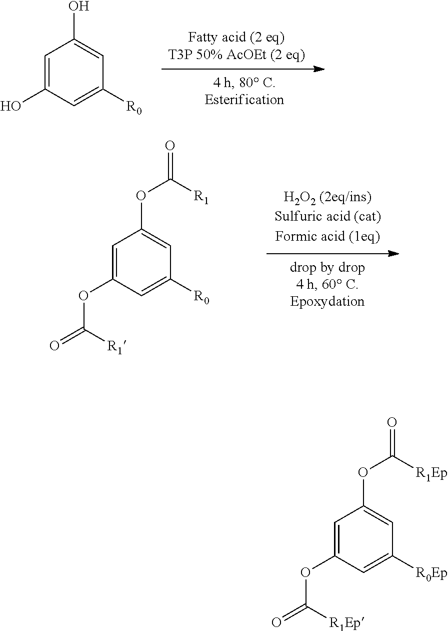

A third object of the present invention is a plastic material comprising cellulose acetate obtainable by a process according to the invention as defined above.

A fourth object of the present invention is a compound of formula (1)

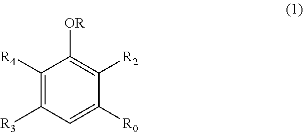

formula (1) in which
R is selected from
   a hydrogen atom
   a CO—R₁ group,
R₀ is selected from
   a linear alkyl group of 15 carbon atoms, optionally containing 1 to 3 epoxide functions or,
   a linear alkenyl group of 15 carbon atoms, comprising x alkenyl functions, x varying from 1 to 3, comprising y epoxide functions, y varying from 0 to 2, x+y being less than or equal to 3,
R₂ is selected from
   a hydrogen atom,
   —CO—O—R₅ of formula

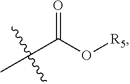

a carboxylic acid function COOH
R₃ are independently selected from:
   a hydrogen atom,
   —OC—O—R₆ with formula

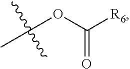

an OH alcohol function,
R₄ is selected from
   a hydrogen atom,
   a methyl group CH₃,
R₁, R₅ and R₆ are identical or different and are selected from
   a linear alkyl group of 2 to 30 carbon atoms, optionally containing 1 to 6 epoxide functions, or
   a linear alkenyl group of 2 to 24 carbon atoms, optionally comprising 1 to 6 epoxide function(s), said alkenyl group being optionally substituted by an alkyl group of 1 to 5 carbon atom(s),
      R₀, R₁, R₅ and R₆ optionally comprising one to six substituent(s), in particular one or two, selected from —OH, —OR₇ or OCOR₇, wherein R₇ is an alkyl group of 1 to 3 carbon atoms,
      with the proviso that at least one of the OR, R₂ or R₃ groups is an ester function and/or at least one of R₀, R₁, R₅ or R₆ comprises at least one epoxide function and with the proviso that when the compound of formula (1) is:

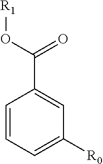

then the compound has at least one epoxide function
the following compounds are excluded:

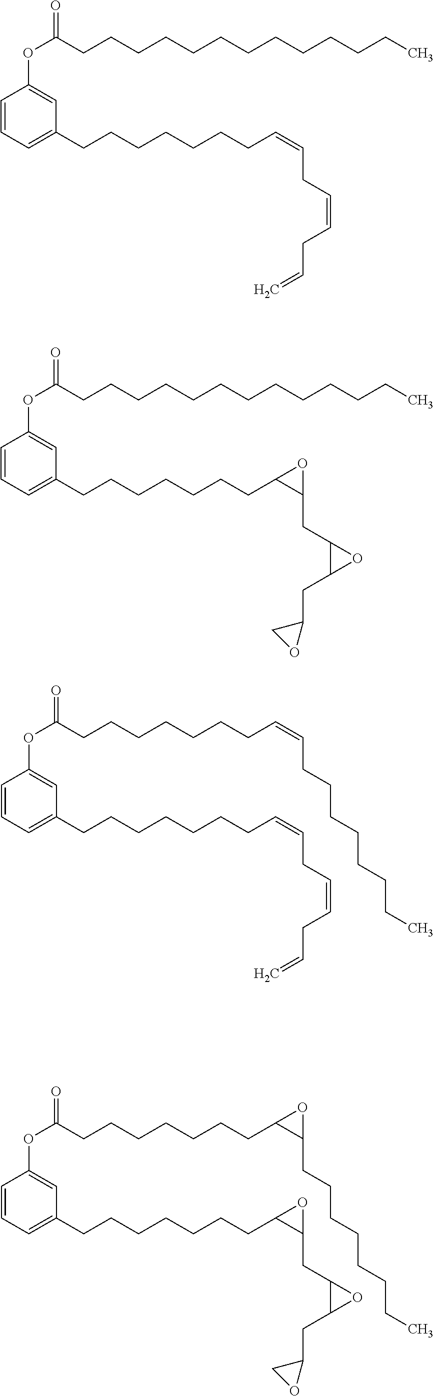 [SMILES: C=CC/C=C\C/C=C\CCCCCCCc1cccc(OC(=O)CCCCCCCCCCCCC)c1 ; CCCCCCCCCCCCCC(=O)Oc1cccc(CCCCCCCC2OC2CC2OC2CC2CO2)c1 ; C=CC/C=C\C/C=C\CCCCCCCc1cccc(OC(=O)CCCCCCC/C=C\CCCCCCCC)c1 ; CCCCCCCCC1OC1CCCCCCCC(=O)Oc1cccc(CCCCCCCC2OC2CC2OC2CC2CO2)c1]

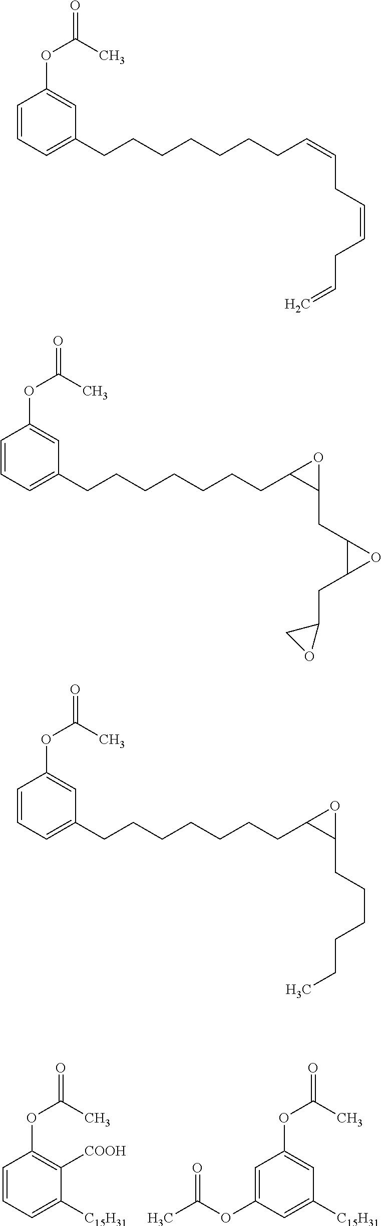

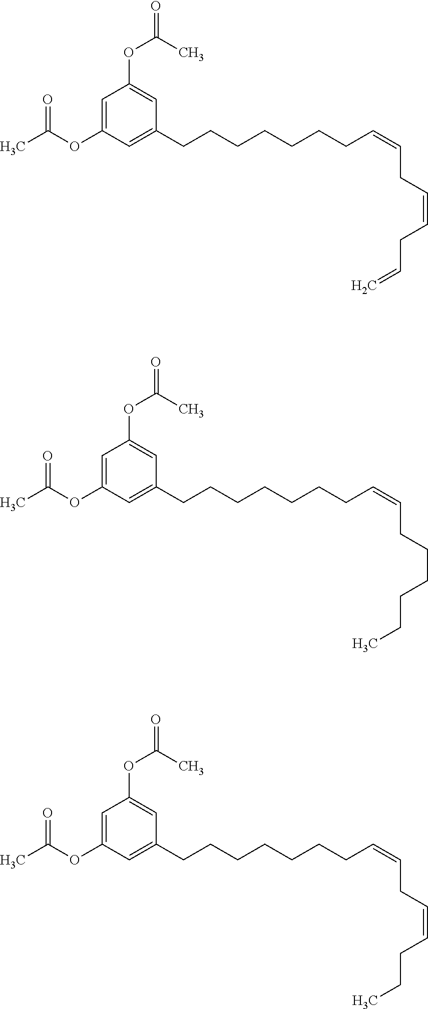 [SMILES: C=CC/C=C\C/C=C\CCCCCCCc1cc(OC(C)=O)cc(OC(C)=O)c1 ; CCCCCC/C=C\CCCCCCCc1cc(OC(C)=O)cc(OC(C)=O)c1 ; CCC/C=C\C/C=C\CCCCCCCc1cc(OC(C)=O)cc(OC(C)=O)c1]

According to another particular embodiment, the present invention relates to a compound as defined above, of formula selected from the following formulae (3), (4), (5) and (6),

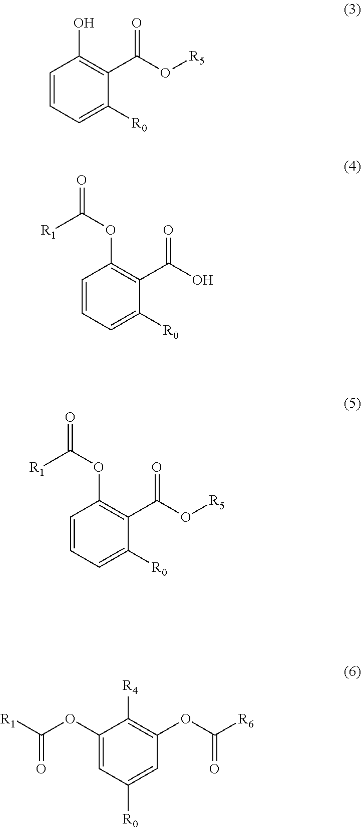

R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₄ is selected from:
a hydrogen atom,
a methyl group CH₃
   R₁, R₅ and R₆ are selected from:
a linear alkyl group of 2 to 30 carbon atom(s), or
a linear alkenyl group of 2 to 24 carbon atoms, said alkenyl group being optionally substituted by an alkyl group of 1 to 5 carbon atoms,
   in particular where:
R₀ is selected from

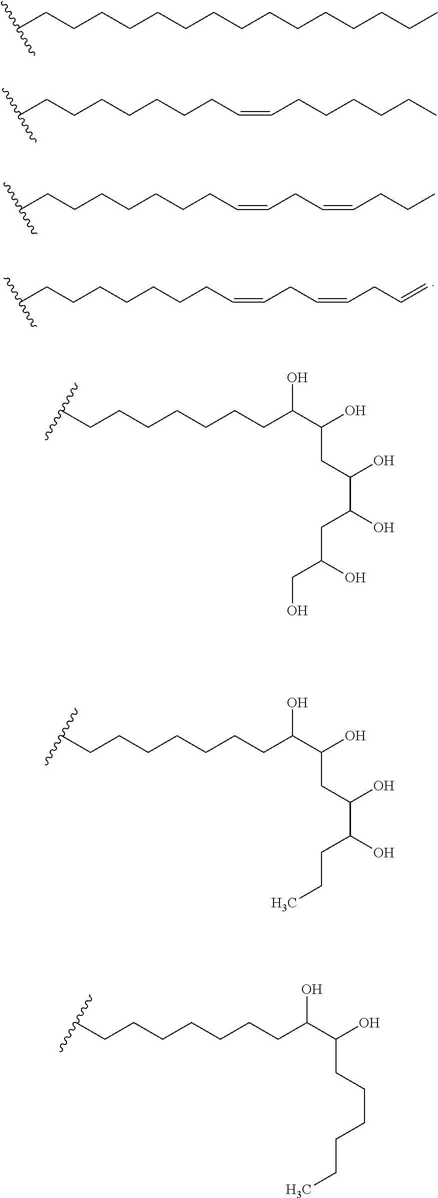

said R₀ group optionally comprising 1 to 6 substituent(s) selected from —OH, —OR₇ and OCOR₇, wherein R₇ is an alkyl group of 1 to 3 carbon atom(s)
said R₁, R₅ and R₆ groups optionally comprising 1 to 6 substituent(s), in particular one or two, selected from —OH, —OR₇ and OCOR₇.

According to another particular embodiment, the present invention relates to a compound as defined above, of formula selected from the following formulae (8) and (9),

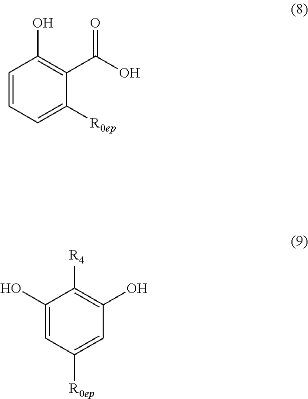

R₀ₑₚ represents:
a linear alkyl group of 15 carbon atoms containing from 1 to 3 epoxide functions, or
a linear alkenyl group of 15 carbon atoms, comprising x alkenyl functions, x varying from 1 to 2, comprising y epoxide functions, y varying from 1 to 2, x+y being less than or equal to 3, said group R₀ₑₚ optionally comprising 1 to 6 substituent(s) selected from —OH, —OR₇ and OCOR₇, wherein R₇ is an alkyl group of 1 to 3 carbon atom(s).
   in particular where:
   R₀ₑₚ is selected from:

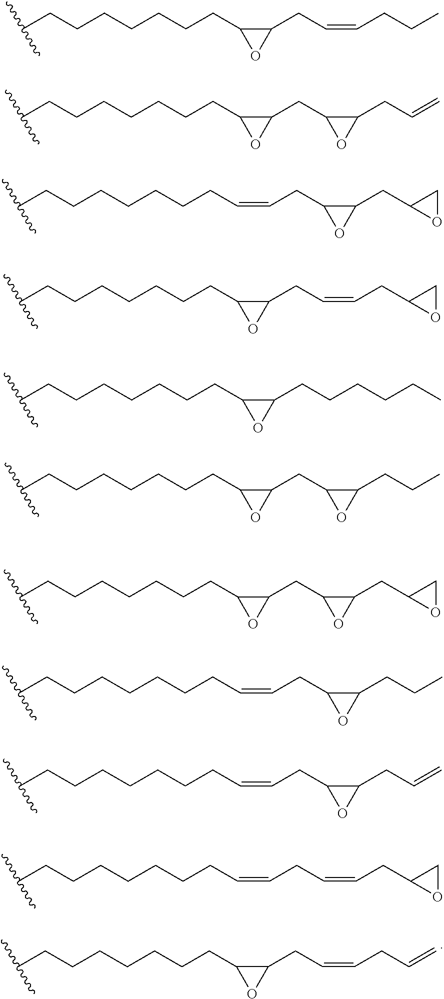

According to another particular embodiment, the present invention relates to a compound as defined above, selected from the following formulae (10), (11), (12), (13) and (14),

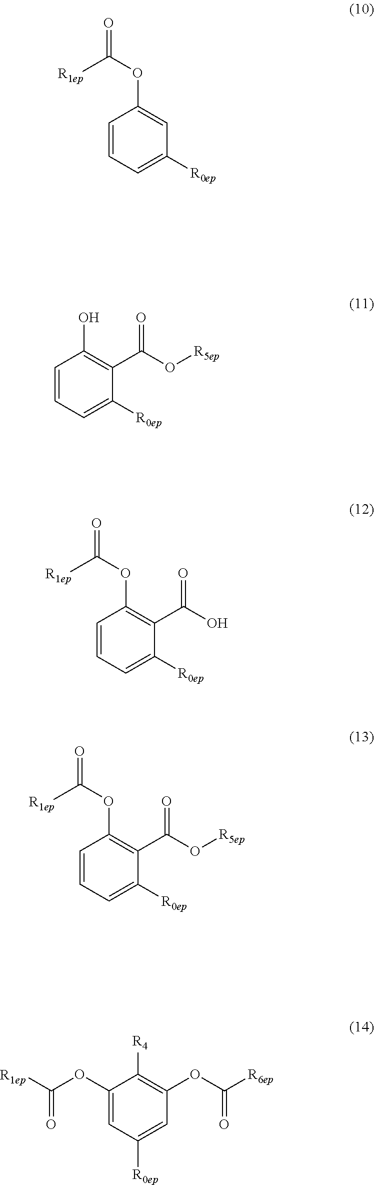

R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₄ is selected from:
a hydrogen atom,
a methyl group CH₃
   R₁ₑₚ, R₅ₑₚ and R₆ₑₚ are independently selected from:
an alkyl group of 2 to 30 carbon atoms, optionally containing 1 to 6 epoxide functions,
a linear alkenyl group of 2 to 24 carbon atoms, in particular comprising 1 to 6 epoxide functions, said alkenyl group being optionally substituted by an alkyl chain with 1 to 5 carbon atoms,
   with the proviso that at least one of the R₀ₑ, R₁ₑₚ, R₅ₑₚ or R₆ₑₚ groups contains at least one epoxide function,
   in particular where
R₀ₑₚ is selected from

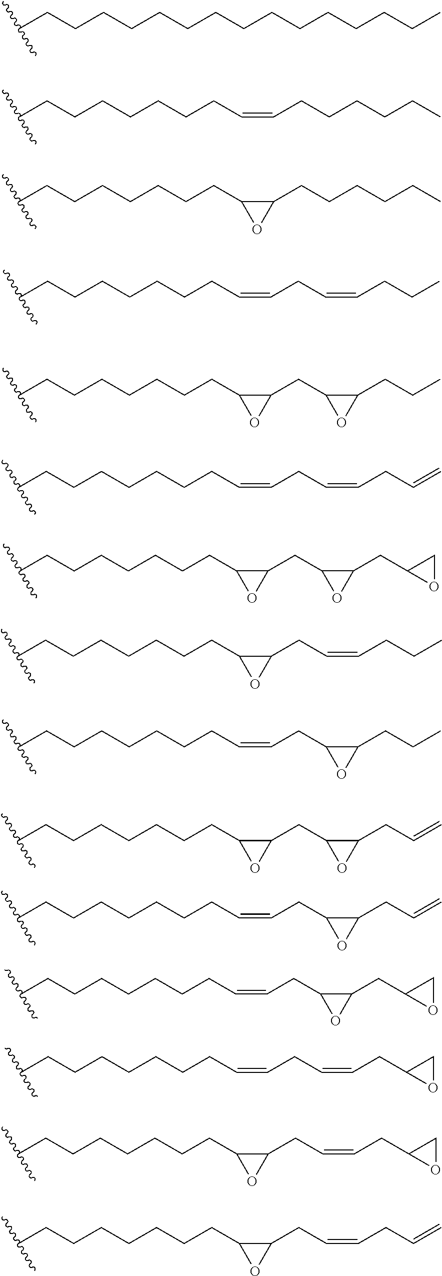

said group R₀ₑₚ optionally comprising 1 to 6 substituent(s) selected from —OH, —OR₇ and OCOR₇, wherein R₇ is an alkyl group of 1 to 3 carbon atom(s)
said R₁ₑₚ, R₅ₑₚ and R₆ₑₚ groups optionally comprising 1 to 6 substituent(s), in particular one or two, selected from —OH, —OR₇ and OCOR₇.

According to another particular embodiment, the present invention relates to a compound as defined above, selected from the following formulae (11), (12), (13) and (14),

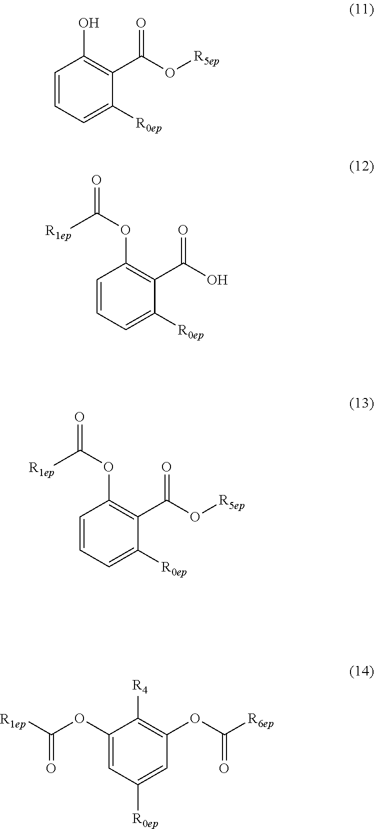

R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₄ is selected from:
a hydrogen atom,
a methyl group CH₃
   R₁ₑₚ, R₅ₑₚ and R₆ₑₚ are independently selected from:
an alkyl group of 2 to 30 carbon atoms, optionally containing 1 to 6 epoxide functions,
a linear alkenyl group, with 2 to 24 carbon atoms, in particular comprising 1 to 6 epoxide functions, said alkenyl group being optionally substituted by an alkyl chain with 1 to 5 carbon atoms,
   with the proviso that at least one of the R₀ₑₚ, R₁ₑₚ, R₅ₑₚ or R₆ₑₚ groups contains at least one epoxide function,
   in particular where
R₀ₑₚ is selected from:

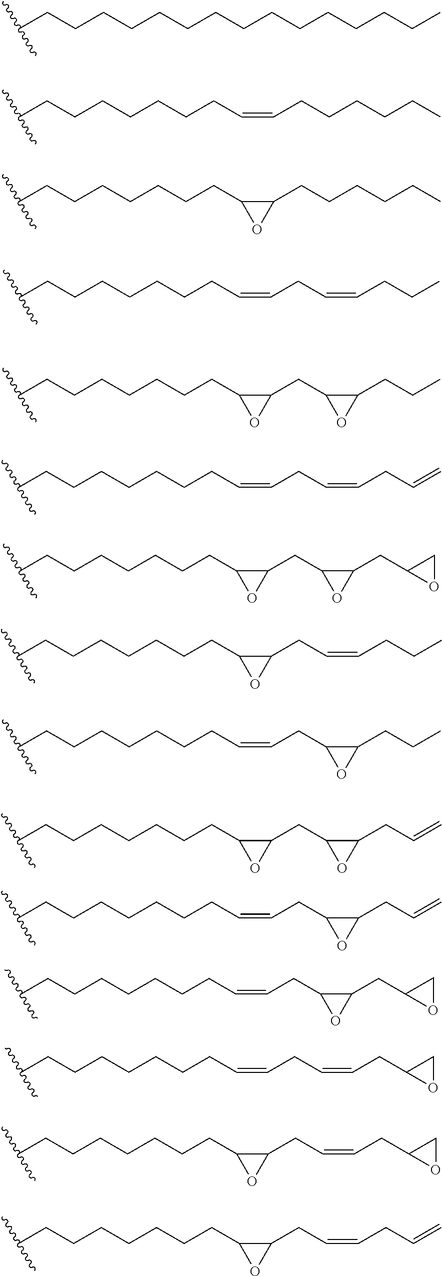

said group R₀ₑₚ optionally comprising 1 to 6 substituent(s) selected from —OH, —OR₇ and OCOR₇, wherein R₇ is an alkyl group of 1 to 3 carbon atom(s)
said R₁ₑₚ, R₅ₑₚ and R₆ₑₚ groups optionally comprising 1 to 6 substituent(s), in particular one or two, selected from —OH, —OR₇ and OCOR₇.

According to another particular embodiment, the present invention relates to a compound as defined above, of formula (10)

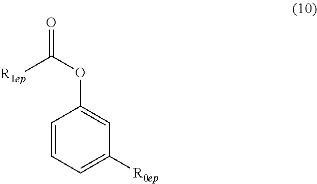

formula (2) in which
R₀ₑₚ is selected from
   a linear alkyl group of 15 carbon atoms optionally containing 1 to 3 epoxide functions, or
   a linear alkenyl group of 15 carbon atoms, comprising x alkenyl functions, x varying from 1 to 3, comprising y epoxide functions, y varying from 0 to 2, x+y being less than or equal to 3,
R₁ₑₚ is selected from
   a linear alkyl group of 3 to 13 or 15 to 30 carbon atoms, optionally containing 1 to 6 epoxide functions, or
   a linear alkenyl group of 2 to 17 or 19 to 24 carbon atoms, optionally comprising 1 to 6 epoxide function(s), said alkenyl group being optionally substituted by an alkyl group of 1 to 5 carbon atom(s),
      R₀ₑₚ optionally comprising one to six substituent(s) selected from —OH, —OR₇ or OCOR₇, wherein R₇ is an alkyl group of 1 to 3 carbon atoms,
      R₁ₑₚ optionally comprising one to six substituent(s), in particular one or two selected from —OH, —OR₇ or OCOR₇, wherein R₇ is an alkyl group of 1 to 3 carbon atoms,
      with the proviso that R₀ₑₚ or R₁ₑₚ contains at least one epoxide function.

According to another particular embodiment, the present invention relates to a compound as defined above, of formulae selected from formulae (2), (3), (4), (5) and (6), in which:
R₀ is selected from

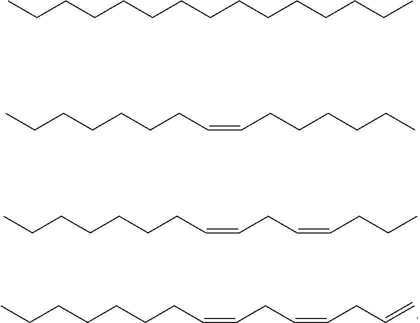

According to another particular embodiment, the present invention relates to a compound as defined above, of formulae selected from formulae (7), (8) and (9),

in which:
R₀ₑₚ is selected from

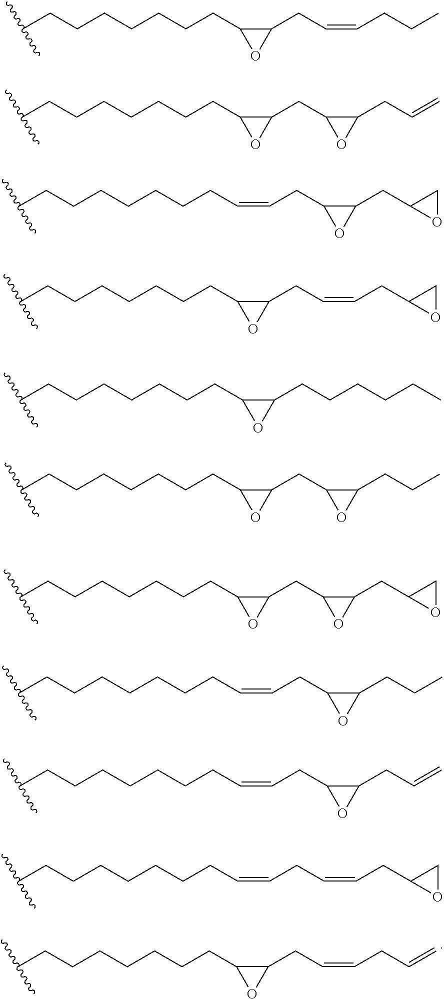

According to another particular embodiment, the present invention relates to a compound as defined above, of formulae selected from formulae (10), (11), (12), (13) and (14), in which:
R₀ₑₚ is selected from:

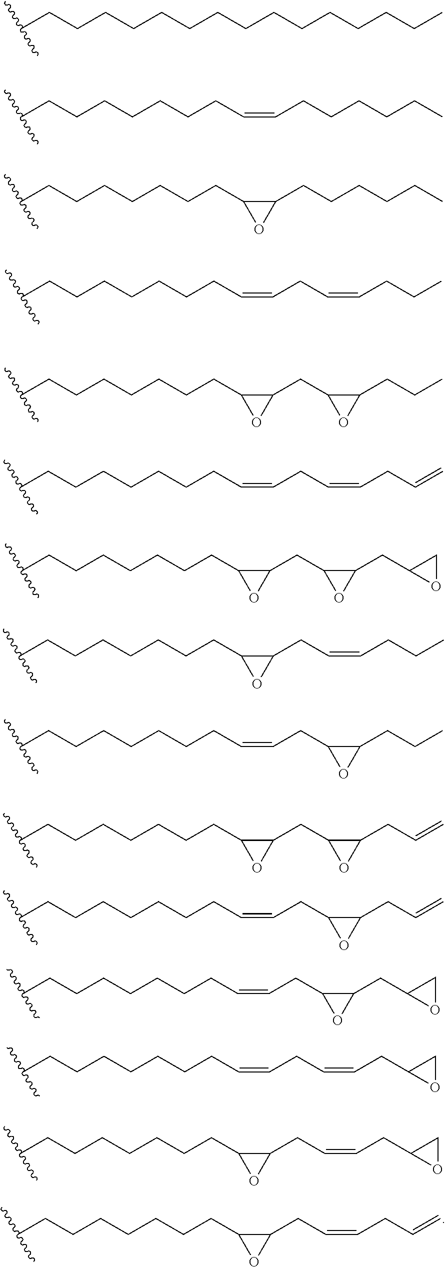

A fifth object of the present invention is a process for preparing a compound of formula (1) as defined above according to the invention.

According to another particular embodiment, the present invention relates to a process for the preparation of a compound of formula (2) as defined above, comprising a step of esterification, for example using 4-dimethylaminopyridine and 1-ethyl-3-(3-dimethylaminopropyl)carbodiimide or propylphosphonic anhydride, of a compound of formula (15) with an acid of formula (16):

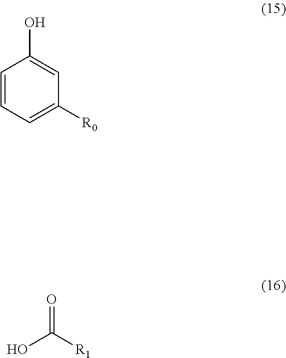

formula (15) in which R₀ represents
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3, formula (16) in which R₁ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   R₁ optionally comprising one to six substituents, in particular one or two, selected from —OH, —OR₇ or —OCOR₇, wherein R₇ is an alkyl group of 1 to 3 carbon atoms, to obtain the compound of formula (2)

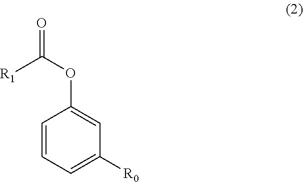

in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3.
   R₁ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   R₁ optionally comprising one to six substituent(s), in particular one or two, selected from —OH, —OR₇ or —OCOR₇, wherein R₇ is an alkyl group of 1 to 3 carbon atoms.

According to another particular embodiment, the present invention relates to a process for the preparation of a compound of formula (3) as defined above, comprising a step of esterification, for example with sulfuric acid, of a compound of formula (17) with an alcohol of formula (18)

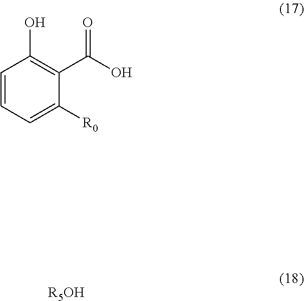

formula (18) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3, formula (18) in which R₅ represents:
a linear alkyl group of 1 to 30 carbon atom(s), or
a linear alkenyl group of 2 to 24 carbon atoms, comprising 1 to 6 alkenyl functions, said alkenyl group being optionally substituted by a linear alkyl group of 1 to 5 carbon atoms,
   R₅ optionally comprising one to six substituents, in particular one or two, selected from —OH, —OR₇ or OCOR₇, wherein R₇ is an alkyl group of 1 to 3 carbon atoms,

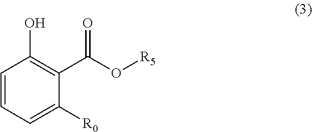

to obtain the compound of formula (3) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₅ represents:
a linear alkyl group of 1 to 30 carbon atom(s), or
a linear alkenyl group of 2 to 24 carbon atoms, comprising 1 to 6 alkenyl functions, said alkenyl group being optionally substituted by a linear alkyl group of 1 to 5 carbon atoms,
   R₅ optionally comprising one to six substituents, in particular one or two, selected from —OH, —OR₇ or OCOR₇, wherein R₇ is an alkyl group of 1 to 3 carbon atoms.

According to another particular embodiment, the present invention relates to a process for the preparation of a compound of formula (4) as defined above, comprising a step of esterification, for example using 4-dimethylaminopyridine and 1-ethyl-3-(3-dimethylaminopropyl)carbodiimide or propylphosphonic anhydride, of a compound of formula (17) with an acid of formula (19),

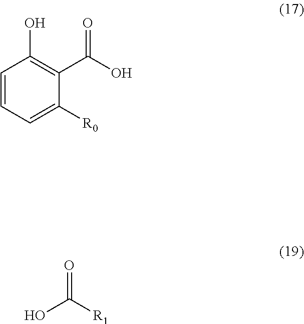

formula (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3.
   formula (19) in which R_{1B} represents
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s), said R₁ group optionally comprising one to six substituent(s), in particular one or two, selected from —OH, —OR₇ and OCOR₇, wherein R₇ is an alkyl group of 1 to 3 carbon atom(s), to obtain the compound of formula (4)

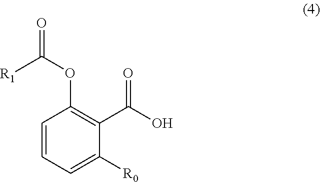

in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3.
   R₁ represents
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s), said R₁ group optionally comprising one to six substituent(s), in particular one or two, selected from —OH, —OR₇ and OCOR₇, wherein R₇ is an alkyl group of 1 to 3 carbon atom(s).

According to another particular embodiment, the present invention relates to a process for preparing a compound of formula (5) as defined above, comprising a step of esterifying a compound of formula (17) with an alcohol of formula (18) and an acid of formula (19):

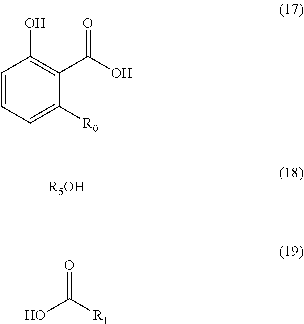

formula (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3, formulae (18) and (19) in which R₁ and R₅ independently represent:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s), said R₁ and R₅ groups, in addition to the —OH group represented in formula (18), optionally comprising one to six substituent(s), in particular one or two, selected from —OH, —OR₇ and OCOR₇, wherein R₇ is an alkyl group of 1 to 3 carbon atom(s),
   comprising the following steps:
i) a first step of esterification, for example with sulfuric acid, of the compound of formula (17) with a compound (18) to obtain a compound of formula (3),

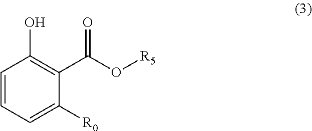

formula (3) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3.
   R₅ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   said R₅ group optionally comprising one to six substituent(s), in particular one or two, selected from —OH, —OR₇ and OCOR₇, wherein R₇ is an alkyl group of 1 to 3 carbon atom(s),
ii) a second step of esterification, for example using 4-dimethylaminopyridine and 1-ethyl-3-(3-dimethylaminopropyl)carbodiimide or propylphosphonic anhydride, of the compound of formula (3) with a compound of formula (19) to obtain the compound of formula (5),

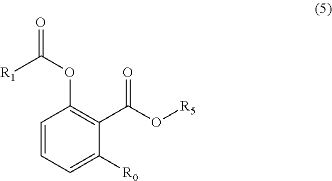

formula (5) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₁ and R₅ independently represent:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   said R₁ and R₅ groups optionally comprising one to six substituent(s), in particular one or two, selected from —OH, —OR₇ and OCOR₇, wherein R₇ is an alkyl group of 1 to 3 carbon atom(s).

According to another particular embodiment, the present invention relates to a process for the preparation of a compound of formula (6) as defined above, comprising a step of esterification, for example with 4-dimethylaminopyridine and 1-ethyl-3-(3-dimethylaminopropyl)carbodiimide or propylphosphonic anhydride of a compound of formula (20) with an acid of formula (21) and an acid of formula (22)

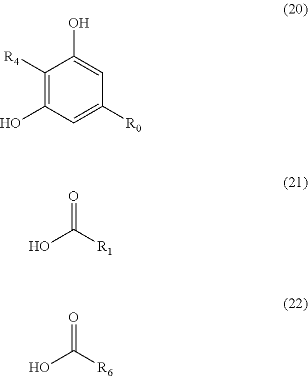

formula (20) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   in which R₄ represents:
a hydrogen atom
a methyl group CH₃,
   formulae (21) and (22) in which R₁ and R₆ independently represent, in particular may be identical:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   comprising a step of esterifying the compound of formula (20) with an acid of formula (21) and an acid of formula (22), in particular the fatty acids of formulae (21) and (22) are identical, to obtain the compound of formula (6),
   said R₁ and R₆ groups optionally comprising one to six, in particular one or two, substituent(s) selected from —OH, —OR₇ and OCOR₇, wherein R₇ is an alkyl group of 1 to 3 carbon atom(s),

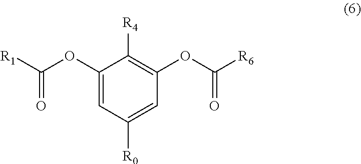

in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3, in which R₄ represents:
a hydrogen atom,
a methyl group CH₃,
   R₁ and R₆ independently represent, in particular may be identical:
a linear alkyl group of 1 to 24 carbon atoms, or
a linear alkenyl group of 2 to 24 carbon atoms, comprising 1 to 6 alkenyl function(s), said alkenyl group being optionally substituted by a linear alkyl group of 1 to 5 carbon atom(s), said R₁ and R₆ groups optionally comprising one to six substituent(s), in particular one or two, selected from —OH, —OR₇ and OCOR₇, wherein R₇ is an alkyl group of 1 to 3 carbon atom(s).

According to another particular embodiment, the present invention relates to a process for preparing a compound of formula (7) as defined above comprising an epoxidation step of a compound of formula (15) and optionally a diol formation step of the compound of formula (7):

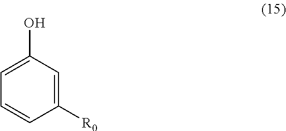

formula (15) in which R₀ represents:
a linear alkenyl group of 15 carbon atoms, comprising x alkenyl functions, x varying from 1 to 3, comprising y epoxide functions, y varying from 0 to 2, x+y being less than or equal to 3, to obtain the compound of formula (7)

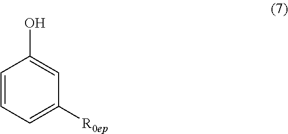

in which R₀ₑₚ represents:
a linear alkyl group of 15 carbon atoms containing from 1 to 3 epoxide functions, or
a linear alkenyl group of 15 carbon atoms, comprising x alkenyl functions, x varying from 1 to 2, comprising y epoxide functions, y varying from 1 to 2, x+y being less than or equal to 3 R₀ₑₚ optionally comprising one to six substituent(s) selected from —OH, —OR₇ or —OCOR₇, wherein R₇ is an alkyl group of 1 to 3 carbon atoms,
   comprising the following steps:
i) an epoxidation step of a compound of formula (15), for example using formic acid, hydrogen peroxide and sulfuric acid to obtain the compound of formula (7)

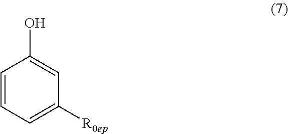

in which R₀ₑₚ represents:
   a linear alkyl group of 15 carbon atoms containing from 1 to 3 epoxide functions, or
   a linear alkenyl group of 15 carbon atoms, comprising x alkenyl functions, x varying from 1 to 2, comprising y epoxide functions, y varying from 1 to 2, x+y being less than or equal to 3
ii) optionally, a diol(s) formation step, for example using formic acid, hydrogen peroxide and sulfuric acid to obtain the compound of formula (7)

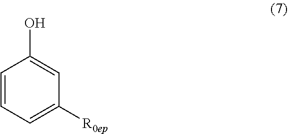

in which R₀ₑₚ represents:
   a linear alkyl group of 15 carbon atoms containing from 1 to 3 epoxide functions, or
   a linear alkenyl group of 15 carbon atoms, comprising x alkenyl functions, x varying from 1 to 2, comprising y epoxide functions, y varying from 1 to 2, x+y being less than or equal to 3
R₀ₑₚ comprising two to six substituent(s) selected from —OH, —OR₇ or —OCOR₇, wherein R₇ is an alkyl group of 1 to 3 carbon atoms,

According to another particular embodiment, the present invention relates to a process for preparing a compound of formula (8) as defined above comprising an epoxidation step of a compound of formula (17) and optionally a diol formation step of the compound of formula (8):

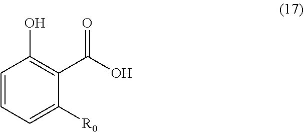

formula (17) in which R₀ represents
a linear alkenyl group of 15 carbon atoms, comprising x alkenyl functions, x varying from 1 to 3, comprising y epoxide functions, y varying from 0 to 2, x+y being less than or equal to 3, to obtain the compound of formula (8),

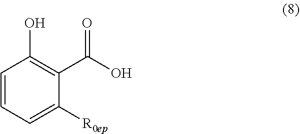

formula (8) in which R₀ₑₚ represents:
a linear alkyl group of 15 carbon atoms containing from 1 to 3 epoxide functions, or
a linear alkenyl group of 15 carbon atoms, comprising x alkenyl functions, x varying from 1 to 2, comprising y epoxide functions, y varying from 1 to 2, x+y being less than or equal to 3.
   R₀ₑₚ optionally comprising one to six substituent(s) selected from —OH, —OR₇ or —OCOR₇, wherein R₇ is an alkyl group of 1 to 3 carbon atoms,
   comprising the following steps:
i) an epoxidation step of a compound of formula (17), for example using formic acid, hydrogen peroxide and sulfuric acid to obtain the compound of formula (8)

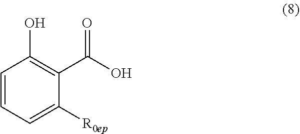

in which R₀ₑₚ represents:
   a linear alkyl group of 15 carbon atoms containing from 1 to 3 epoxide functions, or
   a linear alkenyl group of 15 carbon atoms, comprising x alkenyl functions, x varying from 1 to 2, comprising y epoxide functions, y varying from 1 to 2, x+y being less than or equal to 3
ii) optionally, a diol formation step, for example using formic acid, hydrogen peroxide and sulfuric acid to obtain the compound of formula (8)

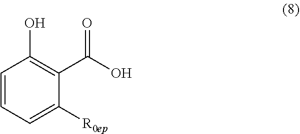

in which R₀ₑₚ represents:
   a linear alkyl group of 15 carbon atoms containing from 1 to 3 epoxide functions, or
   a linear alkenyl group of 15 carbon atoms, comprising x alkenyl functions, x varying from 1 to 2, comprising y epoxide functions, y varying from 1 to 2, x+y being less than or equal to 3
R₀ₑₚ comprising two to six substituent(s) selected from —OH, —OR₇ or —OCOR₇, wherein R₇ is an alkyl group of 1 to 3 carbon atoms,

According to another particular embodiment, the present invention relates to a process for the preparation of a compound of formula (9) as defined above, comprising a step of epoxidation of a compound of formula (20) and optionally a step of diol formation of the compound of formula (9)

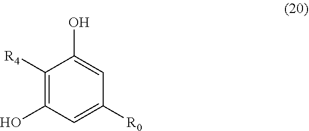

formula (20) in which R₀ represents:
a linear alkenyl group of 15 carbon atoms, comprising x alkenyl functions, x varying from 1 to 3, comprising y epoxide functions, y varying from 0 to 2, x+y being less than or equal to 3,
   in which R₄ represents:
a hydrogen atom
a methyl group CH₃,
   to obtain the compound of formula (9)

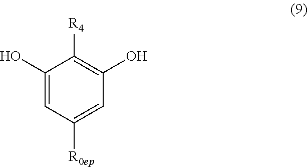

in which R₀ₑₚ represents:
a linear alkyl group of 15 carbon atoms containing from 1 to 3 epoxide functions, or
a linear alkenyl group of 15 carbon atoms, comprising x alkenyl functions, x varying from 1 to 2, comprising y epoxide functions, y varying from 1 to 2, x+y being less than or equal to 3,
   R₀ₑₚ optionally comprising one to six substituent(s) selected from —OH, —OR₇ or —OCOR₇, wherein R₇ is an alkyl group of 1 to 3 carbon atoms,
   in which R₄ represents:
a hydrogen atom
a methyl group CH₃
   comprising the following steps:
i) an epoxidation step of a compound of formula (20), for example with formic acid, hydrogen peroxide and sulfuric acid to obtain the compound of formula (9)

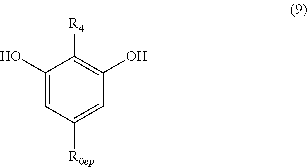

in which R₀ₑₚ represents:
   a linear alkyl group of 15 carbon atoms containing from 1 to 3 epoxide functions, or
   a linear alkenyl group of 15 carbon atoms, comprising x alkenyl functions, x varying from 1 to 2, comprising y epoxide functions, y varying from 1 to 2, x+y being less than or equal to 3
in which R₄ represents:
   a hydrogen atom
   a methyl group CH₃
ii) optionally, a diol(s) formation step, for example using formic acid, hydrogen peroxide and sulfuric acid to obtain the compound of formula (9)

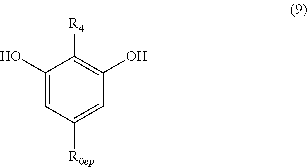

in which R₀ₑₚ represents:
   a linear alkyl group of 15 carbon atoms containing from 1 to 3 epoxide functions, or
   a linear alkenyl group of 15 carbon atoms, comprising x alkenyl functions, x varying from 1 to 2, comprising y epoxide functions, y varying from 1 to 2, x+y being less than or equal to 3
R₀ₑₚ comprising two to six substituent(s) selected from —OH, —OR₇ or —OCOR₇, wherein R₇ is an alkyl group of 1 to 3 carbon atoms,
in which R₄ represents:
   a hydrogen atom
   a methyl group CH₃

According to another particular embodiment, the present invention relates to a process for the preparation of a compound of formula (10) as defined above, from a compound of formula (15) with an acid of formula (16) to obtain a compound of formula (2), followed by epoxidation of the compound of formula (2) and optionally a diol-forming step of the compound of formula (2):

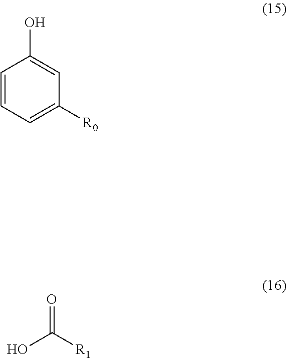

formula (15) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or —OCOR₇, wherein R₇ is an alkyl group of 1 to 3 carbon atoms,
   formula (16) in which R₁ represents:
a linear alkyl group of 1 to 30 carbon atom(s), or
a linear alkenyl group of 2 to 24 carbon atom(s), comprising 1 to 6 alkenyl function(s), said alkenyl group being optionally substituted by a linear alkyl group of 1 to 5 carbon atom(s),
   R₁ optionally comprising one to six substituent(s), in particular one or two, selected from —OH, —OR₇ and OCOR₇, wherein R₇ is an alkyl group of 1 to 3 carbon atom(s),
   with the proviso that at least one of the R₀ or R₁ groups comprises at least one alkenyl function, comprising the following steps:
i) an esterification step, for example using 4-dimethylaminopyridine and 1-ethyl-3-(3-dimethylaminopropyl)carbodiimide or propylphosphonic anhydride of the compound of formula (15) with an acid of formula (16) to obtain a compound of formula (2),

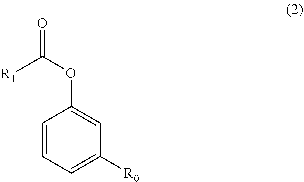

formula (2) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or —OCOR₇, wherein R₇ is an alkyl group of 1 to 3 carbon atoms,
   R₁ represents:
a linear alkyl group of 1 to 30 carbon atom(s), or
a linear alkenyl group of 2 to 24 carbon atom(s), comprising 1 to 6 alkenyl function(s), said alkenyl group being optionally substituted by a linear alkyl group of 1 to 5 carbon atom(s),
   R₁ optionally comprising one to six substituent(s), in particular one or two, selected from —OH, —OR₇ and OCOR₇, wherein R₇ is an alkyl group of 1 to 3 carbon atom(s),
ii) an epoxidation step, for example using formic acid, hydrogen peroxide and sulfuric acid, of the compound of formula (2) to obtain the compound of formula (10)

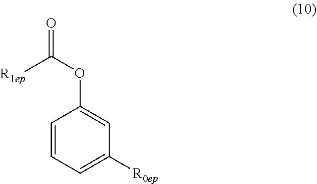

formula (10) in which R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ₑₚ optionally comprising one to six substituent(s) selected from —OH, —OR₇, —OCOR₇, wherein R₇ is an alkyl group of 1 to 3 carbon atoms,
   R₁ₑₚ represents:
a linear alkyl group of 1 to 30 carbon atom(s), optionally containing 1 to 6 epoxide function(s), or
a linear alkenyl group of 2 to 24 carbon atoms, optionally comprising 1 to 6 epoxide function(s), said alkenyl group being optionally substituted by an alkyl group of 1 to 5 carbon atom(s),
   said R₁ₑₚ group optionally comprising one to six substituent(s), in particular one or two, selected from —OH, —OR₇ and OCOR₇, wherein R₇ is an alkyl group of 1 to 3 carbon atom(s),
   with the proviso that at least one of the R₀ₑₚ or R₁ₑₚ groups contains at least one epoxide function.
iii) optionally, a diol formation step, for example using formic acid, hydrogen peroxide and sulfuric acid to obtain the compound of formula (10)

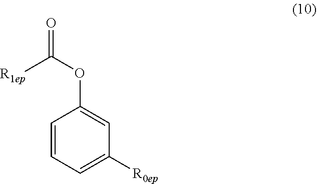

in which R₀ₑₚ represents:
   a linear alkyl group of 15 carbon atoms containing from 1 to 3 epoxide functions, or
   a linear alkenyl group of 15 carbon atoms, comprising x alkenyl functions, x varying from 1 to 2, comprising y epoxide functions, y varying from 1 to 2, x+y being less than or equal to 3
R₀ₑₚ comprising two to six substituent(s) selected from —OH, —OR₇ or —OCOR₇, wherein R₇ is an alkyl group of 1 to 3 carbon atoms,
R₁ₑₚ represents:
   a linear alkyl group of 1 to 30 carbon atom(s), optionally containing 1 to 6 epoxide function(s), or
   a linear alkenyl group of 2 to 24 carbon atoms, optionally comprising 1 to 6 epoxide function(s), said alkenyl group being optionally substituted by an alkyl group of 1 to 5 carbon atom(s),
said R₁ₑₚ group optionally comprising one to six substituent(s), in particular one or two, selected from —OH, —OR₇ and OCOR₇, wherein R₇ is an alkyl group of 1 to 3 carbon atom(s),

According to another particular embodiment, the present invention relates to a process for the preparation of a compound of formula (11) as above, from a compound of formula (17) with an alcohol of formula (18) to obtain a compound of formula (3) followed by epoxidation of the compound of formula (3), and optionally a diol-forming step of the compound of formula (3)

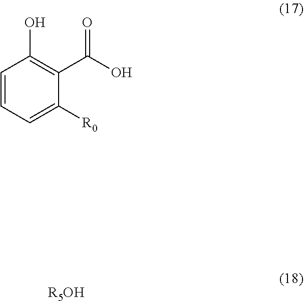

formula (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or —OCOR₇, wherein R₇ is an alkyl group of 1 to 3 carbon atoms,
   formula (18) in which R₅ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s), said R₅ group, in addition to the —OH group represented in formula (18), optionally comprising one to six substituent(s), in particular one or two, selected from —OH, —OR₇ and OCOR₇, wherein R₇ is an alkyl group of 1 to 3 carbon atom(s),
   with the proviso that at least one of the R₀ or R₅ groups comprises at least one alkenyl function, comprising the following steps:
i) a first step of esterification, for example with sulfuric acid, of the compound of formula (17) with a compound (18) to obtain a compound of formula (3),

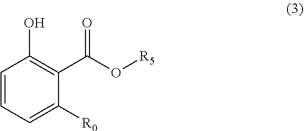

formula (3) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or —OCOR₇, wherein R₇ is an alkyl group of 1 to 3 carbon atoms,
   R₅ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s), said R₅ group optionally comprising one to six substituent(s), in particular one or two, selected from —OH, —OR₇ and OCOR₇, wherein R₇ is an alkyl group of 1 to 3 carbon atom(s), in which at least one of the R₀ or R₅ groups comprises at least one alkenyl function,
ii) an epoxidation step, for example using formic acid, hydrogen peroxide and sulfuric acid, of the compound of formula (3) to obtain the compound of formula (11)

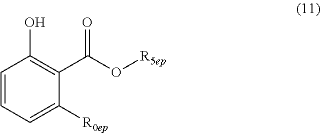

formula (11) in which R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ₑₚ optionally comprising one to six substituent(s) selected from —OH, —OR₇ or —OCOR₇, wherein R₇ is an alkyl group of 1 to 3 carbon atoms,
   R₅ₑₚ represents:
a linear alkyl group of 1 to 30 carbon atom(s), optionally containing 1 to 6 epoxide function(s), or
a linear alkenyl group of 2 to 24 carbon atoms, optionally comprising 1 to 6 epoxide function(s), said alkenyl group being optionally substituted by an alkyl group of 1 to 5 carbon atom(s),
   said R₅ₑₚ groups optionally comprising one to six substituent(s), in particular one or two, selected from —OH, —OR₇ and OCOR₇, wherein R₇ is an alkyl group of 1 to 3 carbon atom(s),
   with the proviso that at least one of the R₀ₑₚ or R₅ₑₚ groups contains at least one epoxide function.
iii) optionally, a diol formation step, for example using formic acid, hydrogen peroxide and sulfuric acid to obtain the compound of formula (11)

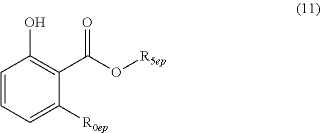

in which R₀ₑₚ represents:
   a linear alkyl group of 15 carbon atoms containing from 1 to 3 epoxide functions, or
   a linear alkenyl group of 15 carbon atoms, comprising x alkenyl functions, x varying from 1 to 2, comprising y epoxide functions, y varying from 1 to 2, x+y being less than or equal to 3
R₀ₑₚ comprising two to six substituent(s) selected from —OH, —OR₇ or —OCOR₇, wherein R₇ is an alkyl group of 1 to 3 carbon atoms,
R₅ₑₚ represents:
   a linear alkyl group of 1 to 30 carbon atom(s), optionally containing 1 to 6 epoxide function(s), or
   a linear alkenyl group of 2 to 24 carbon atoms, optionally comprising 1 to 6 epoxide function(s), said alkenyl group being optionally substituted by an alkyl group of 1 to 5 carbon atom(s),
   said R₅ₑₚ group optionally comprising one to six substituent(s), in particular one or two, selected from —OH, —OR₇ and OCOR₇, wherein R₇ is an alkyl group of 1 to 3 carbon atom(s),

According to another particular embodiment, the present invention relates to a process for the preparation of a compound of formula (12) as defined above, starting from a compound of formula (17) with an acid of formula (19) to obtain a compound of formula (4) followed by epoxidation of the compound of formula (4) and optionally a diol-forming step of a compound of formula (4)

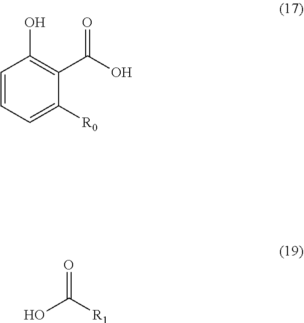

formula (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or —OCOR₇, wherein
   R₇ is an alkyl group of 1 to 3 carbon atoms,
   formula (19) in which R₁ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s), said R₁ group optionally comprising one to six substituent(s), in particular one or two, selected from —OH, —OR₇ and OCOR₇, wherein R₇ is an alkyl group of 1 to 3 carbon atom(s),
   with the proviso that at least one of the R₀ or R₁ groups comprises at least one alkenyl function, comprising the following steps of:
i) an esterification step, for example using 4-dimethylaminopyridine and 1-ethyl-3-(3-dimethylaminopropyl)carbodiimide or propylphosphonic anhydride, of the compound of formula (17) with a compound of formula (19) to obtain the compound of formula (4),

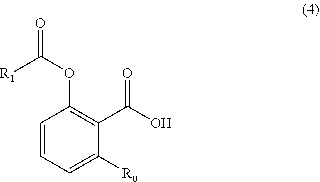

formula (4) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or —OCOR₇, wherein R₇ is an alkyl group of 1 to 3 carbon atoms,
   R₁ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s), said R₁ group optionally comprising one to six substituent(s), in particular one or two, selected from —OH, —OR₇ and OCOR₇, wherein R₇ is an alkyl group of 1 to 3 carbon atom(s), in which at least one of the R₀ or R₁ groups comprises at least one alkenyl function,
   ii) an epoxidation step, for example using formic acid, hydrogen peroxide and sulfuric acid, of the compound of formula (4) to obtain the compound of formula (12),

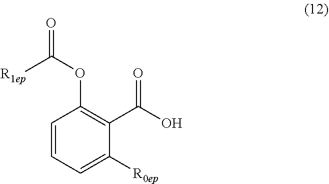

formula (12) in which R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ₑₚ optionally comprising one to six substituent(s) selected from —OH, —OR₇ or —OCOR₇, wherein R₇ is an alkyl group of 1 to 3 carbon atoms,
R₁ₑₚ represents:
   a linear alkyl group of 1 to 30 carbon atom(s), optionally comprising 1 to 6 epoxide function(s), or
a linear alkenyl group of 2 to 24 carbon atoms, optionally comprising 1 to 6 epoxide function(s), said alkenyl group being optionally substituted by an alkyl group of 1 to 5 carbon atom(s),
   said R₁ₑₚ group optionally comprising one to six substituent(s), in particular one or two, selected from —OH, —OR₇ and OCOR₇, wherein R₇ is an alkyl group of 1 to 3 carbon atom(s),
   with the proviso that at least one of the R₀ₑₚ or R₁ₑₚ groups contains at least one epoxide function.
iii) optionally, a diol formation step, for example using formic acid, hydrogen peroxide and sulfuric acid to obtain the compound of formula (12)

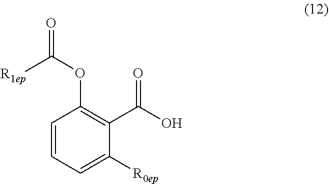

in which R₀ₑₚ represents:
   a linear alkyl group of 15 carbon atoms containing from 1 to 3 epoxide functions, or
   a linear alkenyl group of 15 carbon atoms, comprising x alkenyl functions, x varying from 1 to 2, comprising y epoxide functions, y varying from 1 to 2, x+y being less than or equal to 3
R₀ₑₚ comprising two to six substituent(s) selected from —OH, —OR₇ or —OCOR₇, wherein R₇ is an alkyl group of 1 to 3 carbon atoms,
R₁ₑₚ represents:
   a linear alkyl group of 1 to 30 carbon atom(s), optionally containing 1 to 6 epoxide function(s), or
   a linear alkenyl group of 2 to 24 carbon atoms, optionally comprising 1 to 6 epoxide function(s), said alkenyl group being optionally substituted by an alkyl group of 1 to 5 carbon atom(s),
said R₅ₑₚ group optionally comprising one to six substituent(s), in particular one or two, selected from —OH, —OR₇ and OCOR₇, wherein R₇ is an alkyl group of 1 to 3 carbon atom(s),

According to another particular embodiment, the present invention relates to a process for the preparation of a compound of formula (13) as defined above, from a compound of formula (17) with an alcohol of formula (18) and an acid of formula (19) to obtain a compound of formula (5) followed by epoxidation of the compound of formula (5):

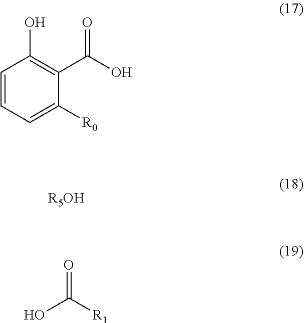

formula (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or —OCOR₇, wherein R₇ is an alkyl group of 1 to 3 carbon atoms,
   formulae (18) and (19) in which R₁ and R₅ independently represent:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   said R₁ and R₅ groups, in addition to the —OH group represented in formula (18), optionally comprising one to six substituent(s), in particular one or two, selected from —OH, —OR₇ and OCOR₇, wherein R₇ is an alkyl group of 1 to 3 carbon atom(s),
   with the proviso that at least one of the R₀, R₁ or R₅ groups comprises at least one alkenyl function,
   comprising the following steps of:
i) a first esterification step, for example using 4-dimethylaminopyridine and 1-ethyl-3-(3-dimethylaminopropyl)carbodiimide or propylphosphonic anhydride or sulfuric acid, of the compound of formula (17) with a compound (18) and a compound (19) to obtain the compound of formula (5),

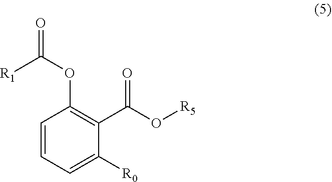

formula (3) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or —OCOR₇, wherein R₇ is an alkyl group of 1 to 3 carbon atoms,
   said R₁ and R₅ groups optionally comprising one to six substituent(s) selected from —OH, —OR₇ and OCOR₇, wherein R₇ is an alkyl group of 1 to 3 carbon atom(s),
   with the proviso that at least one of the R₀, R₁ or R₅ groups comprises at least one alkenyl function,
ii) a second step of epoxidation, for example with formic acid, hydrogen peroxide and sulfuric acid, of the compound of formula (5) to obtain the compound of formula (13)

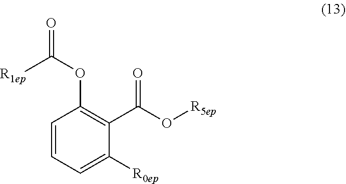

formula (13) in which R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ₑₚ optionally comprising one to six substituent(s) selected from —OH, —OR₇ or —OCOR₇, wherein R₇ is an alkyl group of 1 to 3 carbon atoms,
   R₁ₑₚ and R₅ₑₚ independently represent:
a linear alkyl group of 1 to 30 carbon atom(s), optionally containing 1 to 6 epoxide function(s), or
a linear alkenyl group of 2 to 24 carbon atoms, optionally comprising 1 to 6 epoxide function(s), said alkenyl group being optionally substituted by an alkyl group of 1 to 5 carbon atom(s),
   said R₁ₑₚ and R₅ₑₚ groups optionally comprising one to six substituent(s), in particular one or two, selected from —OH, —OR₇ and OCOR₇,
   with the proviso that at least one of the R₀ₑₚ, R₁ₑₚ or R₅ₑₚ groups contains an epoxide function.
iii) optionally, a diol formation step, for example using formic acid, hydrogen peroxide and sulfuric acid to obtain the compound of formula (13)

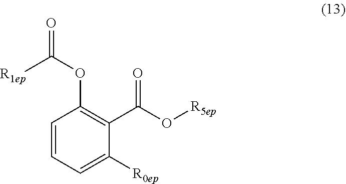

in which R₀ₑₚ represents:
   a linear alkyl group of 15 carbon atoms containing from 1 to 3 epoxide functions, or
   a linear alkenyl group of 15 carbon atoms, comprising x alkenyl functions, x varying from 1 to 2, comprising y epoxide functions, y varying from 1 to 2, x+y being less than or equal to 3
R₀ₑₚ comprising two to six substituent(s) selected from —OH, —OR₇ or —OCOR₇, wherein R₇ is an alkyl group of 1 to 3 carbon atoms,
R₁ₑₚ and R₅ₑₚ independently represent:
   a linear alkyl group of 1 to 30 carbon atom(s), optionally containing 1 to 6 epoxide function(s), or
   a linear alkenyl group of 2 to 24 carbon atoms, optionally comprising 1 to 6 epoxide function(s), said alkenyl group being optionally substituted by an alkyl group of 1 to 5 carbon atom(s),
said R₁ₑₚ and R₅ₑₚ groups optionally comprising one to six substituent(s), in particular one or two, selected from —OH, —OR₇ and OCOR₇, wherein R₇ is an alkyl group of 1 to 3 carbon atom(s),

According to another particular embodiment, the present invention relates to a process for the preparation of a compound of formula (14) as defined above, from a compound of formula (20) with an acid of formula (21) and an acid of formula (22) to obtain a compound of formula (6), followed by an epoxidation of the compound of formula (6) and optionally an epoxidation step of the compound of formula (6):

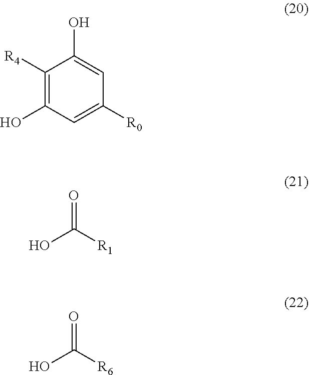

formula (20) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   in which R₄ represents:
a hydrogen atom,
a methyl group CH₃,
   R₀ optionally comprising one to six substituent(s) selected from —OH, —OR₇ or —OCOR₇, wherein R₇ is an alkyl group of 1 to 3 carbon atoms,
   formulae (21) and (22) in which R₁ and R₆ independently represent, in particular may be identical:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   R₁ and R₆ optionally comprising one to six substituent(s), in particular one or two, selected from —OH, —OR₇ and OCOR₇,
   with the proviso that at least one of the R₀, R₁ or R₆ groups comprises at least one alkenyl function,
   comprising the following steps:
i) an esterification step, for example using 4-dimethylaminopyridine and 1-ethyl-3-(3-dimethylaminopropyl)carbodiimide or propylphosphonic anhydride, of the compound of formula (20) with an acid of formula (21) and an acid of formula (22), in particular the fatty acids of formulae (21) and (22) are identical, to obtain the compound of formula (6),

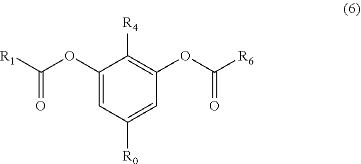

formula (6) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ optionally comprising one to six substituent(s) selected from —OH, —OR₇ or —OCOR₇, wherein R₇ is an alkyl group of 1 to 3 carbon atoms,
   in which R₄ represents:
a hydrogen atom
a methyl group CH₃
   R₁ and R₆ independently represent, in particular may be identical:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
   R₁ and R₆ optionally comprising one to six substituent(s), in particular one or two, selected from —OH, —OR₇ and OCOR₇,
   in which one of the groups comprises at least one alkenyl function,
ii) an epoxidation step, for example using formic acid, hydrogen peroxide and sulfuric acid, of the compound of formula (6) to obtain the compound of formula (14)

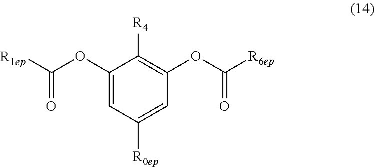

formula (14) in which R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
   R₀ₑₚ optionally comprising one to six substituent(s) selected from —OH, —OR₇ or —OCOR₇, in which R₄ represents:
a hydrogen atom,
a methyl group CH₃,
   R₁ₑₚ and R₆ₑₚ independently represent, in particular may be identical:
a linear alkyl group of 1 to 30 carbon atom(s), optionally containing 1 to 6 epoxide function(s), or
a linear alkenyl group of 2 to 24 carbon atoms, optionally comprising 1 to 6 epoxide function(s), said alkenyl group being optionally substituted by an alkyl group of 1 to 5 carbon atom(s),
   said R₁ₑₚ and R₆ₑₚ groups optionally comprising one to six substituent(s), in particular one or two, selected from —OH, —OR₇ and OCOR₇,
   with the proviso that at least one of the groups R₀ₑₚ, R₁ₑₚ or R₆ₑₚ contains an epoxide function,
iii) optionally, a diol formation step, for example using formic acid, hydrogen peroxide and sulfuric acid to obtain the compound of formula (13)

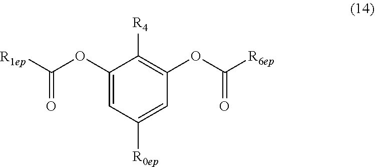

in which R₀ₑₚ represents:
   a linear alkyl group of 15 carbon atoms containing from 1 to 3 epoxide functions, or
   a linear alkenyl group of 15 carbon atoms, comprising x alkenyl functions, x varying from 1 to 2, comprising y epoxide functions, y varying from 1 to 2, x+y being less than or equal to 3
R₀ₑₚ comprising two to six substituent(s) selected from —OH, —OR₇ or —OCOR₇, wherein R₇ is an alkyl group of 1 to 3 carbon atoms,
R₁ₑₚ and R₆ₑₚ independently represent, in particular may be identical:
   a linear alkyl group of 1 to 30 carbon atoms, optionally containing 1 to 6 epoxide functions, or
   a linear alkenyl group of 2 to 24 carbon atoms, optionally comprising 1 to 6 epoxide function(s), said alkenyl group being optionally substituted by an alkyl group of 1 to 5 carbon atom(s),
said R₁ₑₚ and R₆ₑₚ groups optionally comprising one to six substituent(s), in particular one or two, selected from —OH, —OR₇ and OCOR₇
in which R₄ represents:
   a hydrogen atom,
   a methyl group CH₃,

The following examples and FIGURES illustrate the invention, without limiting its scope.

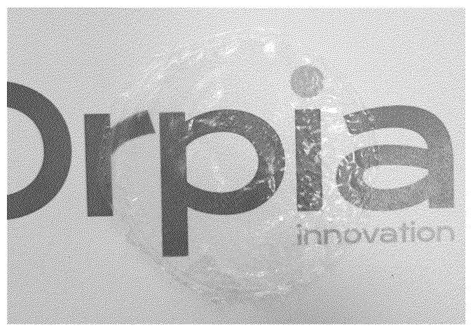

FIG. 1 shows a plastic film obtained with cellulose acetate and plasticizer P1b, of the following formula:

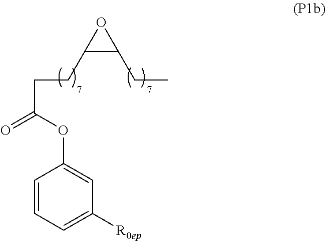

### EXAMPLES

### Materials and Methods

NMR spectra are recorded on a Bruker Avance I 300 MHz instrument. Chemical shifts are counted in parts per million (ppm), where (s) stands for singlet, (d) doublet, (t) triplet, (m) multiplet and (dd) doublet and doublet. For ¹H NMR chemical shifts are measured relative to the residual CDCl₃ peak at 7.26 ppm.

Dynamic mechanical analyses were performed on a Metravib DMA 25 with Dynatest 6.83 software.

Calorimetric analyses were carried out on a Mettler Toledo differential scan calorimeter.

Calorimetric analyses were carried out in a nitrogen atmosphere at a rate of 10° C./min.

Thermal stability of plasticized cellulose acetate was tested on a TA Instruments® Q50 thermogravimetric analyzer.

### Example 1: Cardanol Ester

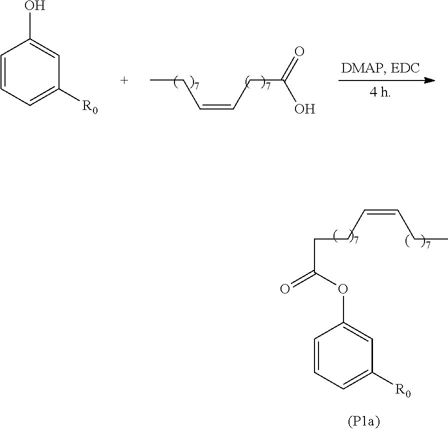

To a flask containing a mixture of cardanol (1 equivalent) and oleic acid (1 equivalent), were added 0.5 equivalents of dimethylamino pyridine (DMAP) and two equivalents of 1-ethyl-3-(3-dimethylaminopropyl)carbodiimide (EDC). The mixture was stirred for 4 hours. The reaction was monitored by thin-layer chromatography. At the end of the reaction, the solvent was evaporated under vacuum. The residue was dissolved in ethyl acetate and washed 3 times with water. The product was purified on a silica chromatography column (95/5 cyclochexane/ethyl acetate mixture). The crude product P1a of the above formula is a colorless/yellowish oil. The yield is 90%.

¹H NMR (CDCl₃), δ (ppm): 0.81-0.91 (m, (2CH₃), 1.2-1.36 (m, CH₂), 1.53-1.62 (m, CH₂), 1.64-1.75 (t, CH₂), 1.93-2.07 (m, CH₂), 2.45-2.53 (t, CH₂) 2.53-2.61 (t, CH₂), 2.71-2.82 (m, CH₂), 4.90-5.05 (m, CH₂═), 5.24-5.42 (m, CH), 5.72-5.86 (m, CH), 6.80-6.87 (m, aromatic CH), 6.99-7.06 (d, aromatic CH), 7.19-7.27 (dd, aromatic CH).

¹³C NMR (CDCl₃), δ (ppm): 14.3 (CH₃), 22.8 (CH₂), 25 (CH₂), 25.8 (CH₂), 27 (CH₂), 27.3 (CH₂), 29.1-29.9 (CH₂), 31.3 (CH₂), 32 (CH₂), 34.6 (CH₂), 35.9 (CH₂), 114.8 (CH₂═), 118.8 (aromatic CH), 121.5 (aromatic CH), 126 (aromatic CH), 129.2 (aromatic CH), 126.9-137 (CH alkyl side chain), 144.7 (C alkyl chain), 150.9 (C—O), 172.5 (O—C═O ester).

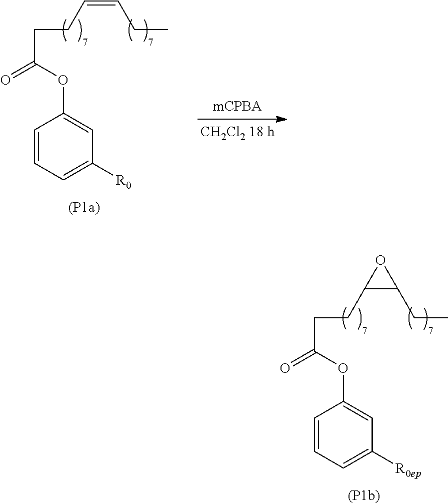

In a two-necked flask fitted with a dropping funnel, 1 equivalent of P1a was dissolved in dichloromethane (DCM) (1 mL for 50 mg of P1). 70% metachloroperbenzoic acid (mCPBA) (2 equivalents) was added dropwise at 0° C. over 5 minutes. The solution was stirred at 0° C. for 1 hour, then at room temperature for 18 h. At the end of the reaction, the product was washed with a saturated aqueous solution of NaHCO₃ and a saturated solution of NaCl. The organic phase was dried with MgSO₄. The solvent was then removed under vacuum. The resulting product P1 b of the above formula is a colorless oil. The yield was 94%.

¹H NMR (CDCl₃), δ (ppm) 0.86-0.96 (m, (2 CH₃), 1.22-1.45 (m, CH₂), 1.47-1.56 (m, CH₂), 1.57-1.68 (m, CH₂), 1.70-1.82 (t, CH₂), 1.97-2.10 (m, CH₂), 2.51-2.57 (t, CH₂) 2.57-2.64 (t, CH₂), 2.79-2.86 (m, CH₂), 2.87-2.94 (m, CH₂), 2.95-3.00 (m, CH), 3.03-3.18 (CH), 6.86-6.94 (m, aromatic CH), 7.00-7.07 (d, aromatic CH), 7.14-7.25 (dd, aromatic CH).

¹³C NMR (CDCl₃), δ (ppm) 14.2 (CH₃), 22.7 (CH₂), 25 (CH₂), 26.2 (CH₂), 26.7 (CH₂), 27.2 (CH₂), 27.9 (CH₂), 29.1-29.7 (CH₂), 31.2 (CH₂), 31.9 (CH₂), 34.5 (CH₂), 35.8 (CH₂), 54.3-57.3 (epoxide) 118.8 (aromatic CH), 121.5 (aromatic CH), 125.9 (aromatic CH), 129.4 (aromatic CH), 144.6 (C-alkyl chain), 150.8 (C—O), 172.3 (O—C═O ester).

### Example 2 Cardanol Ester

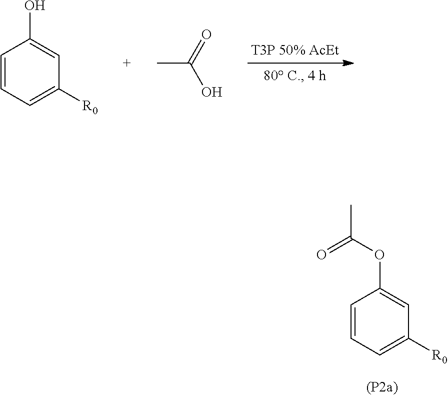

In a flask, a mixture of cardanol (1 equivalent) and acetic acid (1 equivalent) were added to 1 equivalent of propylphosphonic anhydride at 80° C. The reaction was monitored by thin-layer chromatography. At the end of the reaction, the solvent was evaporated under vacuum. The residue was dissolved in ethyl acetate and washed 3 times with water. The crude product P2a of the above formula is a colorless/yellowish oil. The yield was 64%.

¹H NMR (CDCl₃), δ (ppm): 0.82-0.91 (m, (CH₃), 1.2-1.35 (m, CH₂), 1.52-1.64 (m, CH₂), 1.93-2.05 (m, CH₂), 2.30 (s, CH₃), 2.53-2.60 (t, CH₂), 2.71-2.82 (m, CH₂), 4.92-5.06 (m, CH₂═), 5.24-5.42 (m, CH), 5.71-5.86 (m, CH), 6.84-6.88 (m, aromatic CH), 6.98-7.03 (d, aromatic CH), 7.20-7.23 (dd, aromatic CH)).

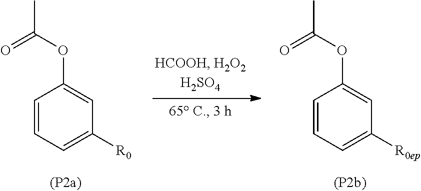

1 equivalent P2a, formic acid (3 equivalents) and sulfuric acid (0.01 equivalents) were stirred and heated at 50° C. Hydrogen peroxide (2 equivalents) was added dropwise to the mixture, which was stirred and heated at 65° C. for 3 hours. At the end of the reaction, a liquid/liquid extraction step was carried out using water and ethyl acetate. The organic phase was washed three times with water and then dried with MgSO₄. The solvent was then removed under vacuum. The crude product P2b of the above formula was purified by silica column chromatography using a 95:5 cyclohexane/ethyl acetate mixture. The P2b product obtained is a colorless oil. The yield is 96%.

¹H NMR (CDCl₃), δ (ppm) 0.85-0.93 (m, (CH₃), 1.25-1.40 (m, CH₂), 1.45-1.56 (m, CH₂), 1.57-1.68 (m, CH₂), 1.70-1.85 (m, CH₂), 2.28 (s, CH₃), 2.54-2.64 (t, CH₂), 2.78-2.85 (m, CH₂), 2.87-2.92 (m, CH₂), 2.93-3.01 (m, CH), 3.04-3.20 (m, CH), 6.87-6.94 (m, aromatic CH), 7.01-7.07 (d, aromatic CH), 7.20-7.23 (dd, aromatic CH)

### Example 3 Esterified Cardanol Diol

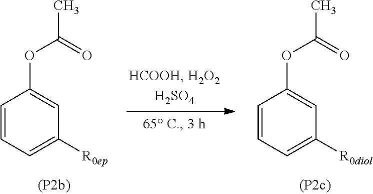

1 equivalent P2b, formic acid (3 equivalents) and sulfuric acid (0.01 equivalents) were stirred and heated at 50° C. Hydrogen peroxide (2 equivalents) was added dropwise to the mixture, which was stirred and heated at 65° C. for 3 hours. At the end of the reaction, a liquid/liquid extraction step was carried out using water and ethyl acetate. The organic phase was washed three times with water, then with a 10% by mass solution of sodium metabisulfite in water, then with a NaCl-saturated water solution, then dried with MgSO₄ and filtered. The solvent was then removed under vacuum. The P2c crude product corresponds to the P2b product in which all epoxide functions have been replaced by diols.

### Example 4: Cardanol Acrylate Ester

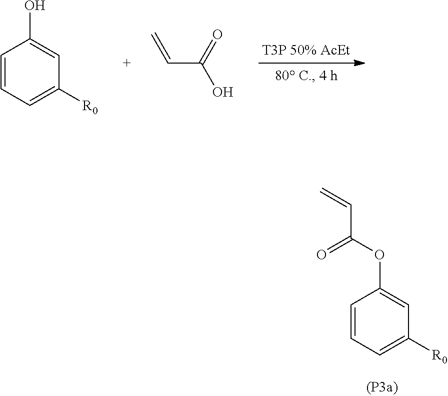

In a flask containing a mixture of cardanol (1 equivalent) and acrylic acid (1 equivalent), 1 equivalent of propylphosphonic anhydride was added at 80° C. The reaction was monitored by thin-layer chromatography. At the end of the reaction, the solvent was evaporated under vacuum. The residue was dissolved in ethyl acetate and washed 3 times with water. The crude product P3a of the above formula is a colorless/yellowish oil. The yield is 45%.

¹H NMR (CDCl₃), δ (ppm): 7.31-7.26 (m, 1H), 7.08-7.03 (dd, 1H), 6.97-6.91 (m, 2H), 6.63-6.56 (ddd, 1H), 6.36-6.27 (m, 1H), 6.03-5.98 (m, 1H), 5.88-5.75 (m, 1H), 5.48-5.28 (m, 1H), 5.08-4.95 (m, 1H), 2.87-2.74 (dt, 2H), 2.64-2.57 (m, 2H), 2.09-1.96 (dt, 2H), 1.68-1.50 (d, 2H), 1.44-1.21 (m, 2H), 0.94-0.85 (dt, 3H).

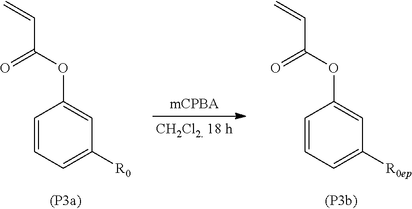

In a two-necked flask fitted with a dropping funnel, 1 equivalent of P3a was dissolved in dichloromethane (DCM) (1 mL for 50 mg of P1). 70% mCPBA (2 equivalents) was added dropwise at 0° C. over 5 minutes. The solution was stirred at 0° C. for 1 hour, then at room temperature for 18 h. At the end of the reaction, the product was washed with a saturated aqueous solution of NaHCO₃ and a saturated solution of NaCl. The organic phase was dried with MgSO₄. The solvent was then removed under vacuum. The resulting product P3b of the above formula is a colorless oil. The yield is 95%.

¹H NMR (CDCl₃), δ (ppm) 7.31-7.26 (m, 1H), 7.10-7.05 (d, 1H), 6.98-6.94 (dt, 2H), 6.63-6.56 (dd, 1H), 6.37-6.27 (dd, 1H), 6.03-5.98 (ddd, 1H), 3.24-2.88 (m, 2H), 2.88-2.78 (m, 2H), 2.69-2.53 (m, 2H), 1.96-1.68 (m, 2H), 1.67-1.56 (m, 2H), 1.56-0.95 (m, 2H), 0.94-0.81 (m, 3H).

¹³C NMR (CDCl₃), δ (ppm) 14.2 (CH₃), 22.7 (CH₂), 25 (CH₂), 26.2 (CH₂), 26.7 (CH₂), 27.2 (CH₂), 27.9 (CH₂), 29.1-29.7 (CH₂), 31.2 (CH₂), 31.9 (CH₂), 34.5 (CH₂), 35.8 (CH₂), 54.3-57.3 (epoxide) 118.8 (aromatic CH), 121.5 (aromatic CH), 125.9 (aromatic CH), 129.4 (aromatic CH), 144.6 (C-alkyl chain), 150.8 (C—O), 172.3 (O—C═O ester).

### Example 5 Cardanol Methacrylate Ester

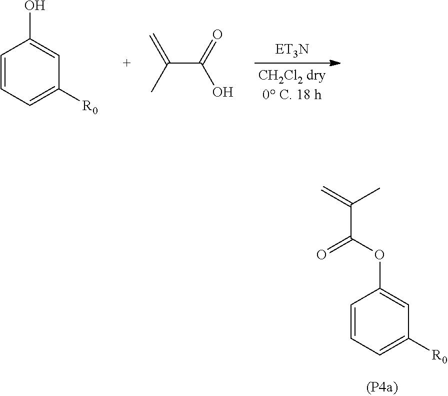

In a sealed flask, cardanol (1 equivalent) was dissolved in dry DCM (3 mL per gram of cardanol). Triethylamine (1 mL per gram of cardanol) was added to the mixture. The solution was purged by bubbling dinitrogen for 30 minutes, then the mixture was cooled in an ice bath. Methacrylic acid was purged for 10 minutes by bubbling dinitrogen for 10 minutes, and added dropwise over 10 minutes (1.7 equivalents). The reaction was stirred for 18 h in an ice bath. The residue was washed 3 times with 0.01 M aqueous NaOH and once with deionized water, then extracted with ethyl acetate. The product was dried with Na₂SO₄, filtered and the solvent evaporated under vacuum. The crude product P4a of the above formula obtained is a colorless/yellowish oil. The yield is 95%.

¹H NMR (CDCl₃), δ (ppm): 0.92 (m, (m, CH₃), 1.21-1.32 (m, CH₂), 1.51-1.59 (m, CH₂), 1.64-1.75 (t, CH₂), 1.93-2.05 (m, CH₂+s, methacrylate CH₃), 2.53-2.61 (t, CH₂), 2.71-2.82 (m, CH₂), 4.95-5.09 (m, CH₂═), 5.24-5.42 (m, CH), 5.57 (s, 1H, methacrylate), 5.75-5.90 (m, CH), 6.17 (s, 1H, methacrylate proton), 6.75-6.85 (m, aromatic CH), 6.99-7.06 (d, aromatic CH), 7.24-7.28 (dd, aromatic CH).

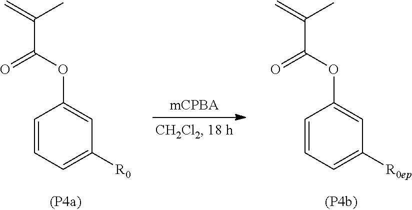

In a two-necked flask fitted with a dropping funnel, 1 equivalent of P4a was dissolved in DCM (1 mL for 50 mg of P1). 70% mCPBA (2 equivalents) was added dropwise at 0° C. over 5 minutes. The solution was stirred at 0° C. for 1 hour, then at room temperature for 18 h. At the end of the reaction, the product was washed with a saturated aqueous solution of NaHCO₃ and a saturated solution of NaCl. The organic phase was dried with MgSO₄. The solvent was then removed under vacuum. The P4b product of the above formula obtained is a yellow oil. The yield was 91%.

¹H NMR (CDCl₃), δ (ppm): 0.94 (m, CH₃), 1.22-1.40 (m, CH₂), 1.47-1.55 (m, CH₂), 1.59-1.65 (m, CH₂), 1.71-1.82 (t, CH₂), 1.97-2.10 (m, CH₂+s, methacrylate CH₃), 2.51-2.58 (t, CH₂) 2.58-2.66 (t, CH₂), 2.79-2.85 (m, CH₂), 2.87-2.94 (m, CH₂), 2.95-3.01 (m, CH), 3.04-3.16 (CH), 4.97-5.96 (remaining alkenyl functions s, 1H, methacrylate), 6.18 (s, 1H, methacrylate proton), 6.86-6.94 (m, aromatic CH), 7.01-7.09 (d, aromatic CH), 7.22-7.29 (dd, aromatic CH).

### Example 6 Epoxidized Cardanol

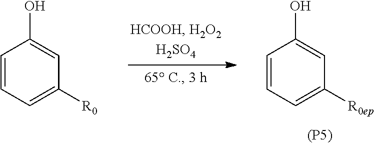

1 equivalent of cardanol, formic acid (3 equivalents) and sulfuric acid (0.01 equivalents) were stirred and heated at 50° C. Hydrogen peroxide (2 equivalents) was added dropwise to the mixture, which was stirred and heated at 65° C. for 3 hours. At the end of the reaction, a liquid/liquid extraction step was carried out using water and ethyl acetate. The organic phase was washed three times with water and then dried with MgSO₄. The solvent was then removed under vacuum. The crude product P5 of the above formula was purified by silica column chromatography using a 95:5 cyclohexane/ethyl acetate mixture. The P5 product obtained is a colorless oil. The yield was 91%.

¹H NMR (CDCl₃), δ (ppm): 0.89-0.91 (m, (CH₃), 1.22-1.45 (m, CH₂), 1.48-1.54 (m, CH₂), 1.57-1.68 (m, CH₂), 1.71-1.82 (t, CH₂), 1.98-2.09 (m, CH₂), 2.51-2.57 (t, CH₂) 2.56-2.64 (t, CH₂), 2.79-2.84 (m, CH₂), 2.87-2.93 (m, CH₂), 2.95-3.00 (m, CH), 3.03-3.18 (CH), 4.97-5.96 (remaining alkenyl functions), 6.64-6.71 (m, aromatic CH), 6.73-6.81 (d, aromatic CH), 7.09-7.16 (dd, aromatic CH).

### Example 7 Esterification and Epoxidation of Cardol

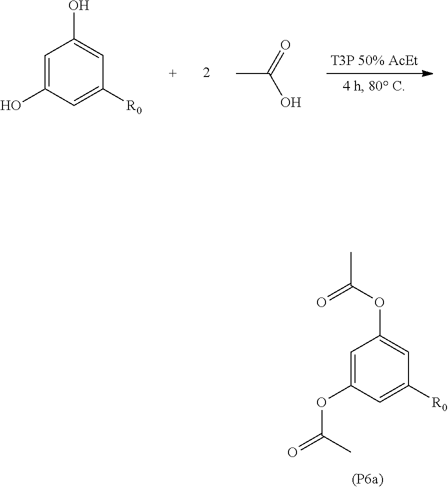

To a flask containing a mixture of cardol (1 equivalent) and acetic acid (2 equivalents), were added 2 equivalents of propylphosphonic anhydride diluted to 50% in ethyl acetate at 80° C. The mixture was stirred for 4 hours. The reaction was monitored by thin-layer chromatography. At the end of the reaction, the solvent was evaporated under vacuum. The residue was dissolved in ethyl acetate and washed 3 times with water. The crude product P6a of the above formula is a colorless/yellowish oil. The yield is 95%.

¹H NMR (CDCl₃), δ (ppm): 6.78 (d, J=2.1 Hz, CH), 6.72 (d, J=2.1 Hz, CH), 5.99-4.96 (m, remaining alkenyl functions), 5.82 (ddt, J=17.2, 10.1, 6.2 Hz, CH), 5.52-5.26 (m, CH), 5.14-4.92 (m, CH), 2.88-2.73 (m, CH₂), 2.63-2.55 (m, CH₂), 2.52 (t, J=7.5 Hz, CH₂), 2.31 (s, CH₃) 2.16-1.95 (m, CH₂), 1.73 (p, J=7.5 Hz, CH₂), 1.60 (t, J=7.5 Hz, CH₂), 1.49-1.18 (m, CH₂), 0.94-0.86 (m, CH)₋₃

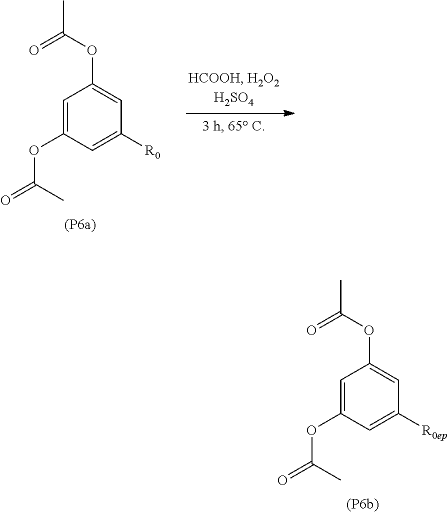

1 equivalent of P6a, formic acid (3 equivalents) and sulfuric acid (0.01 equivalents) were stirred and heated at 50° C. Hydrogen peroxide (2 equivalents) was added dropwise to the mixture, which was stirred and heated at 65° C. for 3 hours. After the reaction was complete, a liquid/liquid extraction step was carried out using water and ethyl acetate. The organic phase was washed three times with water and then dried with MgSO₄. The solvent was then removed under vacuum. The crude product P6b of the above formula was purified by silica column chromatography using a 95:5 cyclohexane/ethyl acetate mixture. The P6b product obtained is a colorless oil. The yield is 91%.

¹H NMR (CDCl₃), δ (ppm): 6.78 (d, J=2.1 Hz, CH), 6.71 (t, J=2.1 Hz, CH), 3.22-3.03 (m, CH), 3.03-2.95 (m, CH), 2.90 (dq, J=6.2, 3.4 Hz, CH₂), 2.65-2.55 (m, CH₂), 2.52 (dd, J=8.6, 6.4 Hz, CH₂), 2.45-2.20 (m, CH₂), 2.29 (s, CH₃) 1.80-1.68 (m, CH₂), 1.67-1.20 (m, CH₂), 0.93-0.84 (m, CH₃).

### Example 8 Esterified Cardol Diol

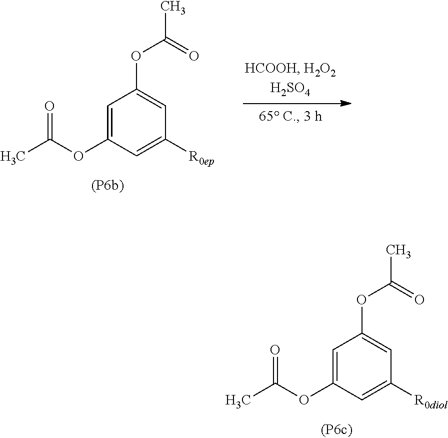

1 equivalent of P6b, formic acid (3 equivalents) and sulfuric acid (0.01 equivalents) were stirred and heated at 50° C. Hydrogen peroxide (2 equivalents) was added dropwise to the mixture, which was stirred and heated at 65° C. for 3 hours. At the end of the reaction, a liquid/liquid extraction step was carried out using water and ethyl acetate. The organic phase was washed three times with water, then with a 10% by mass solution of sodium metabisulfite in water, then with a NaCl-saturated water solution, then dried with MgSO₄ and filtered. The solvent was then removed under vacuum. The P6c crude product corresponds to the P6b product, in which all epoxide functions have been replaced by diols.

### Example 9 Esterification and Epoxidation of Cardol

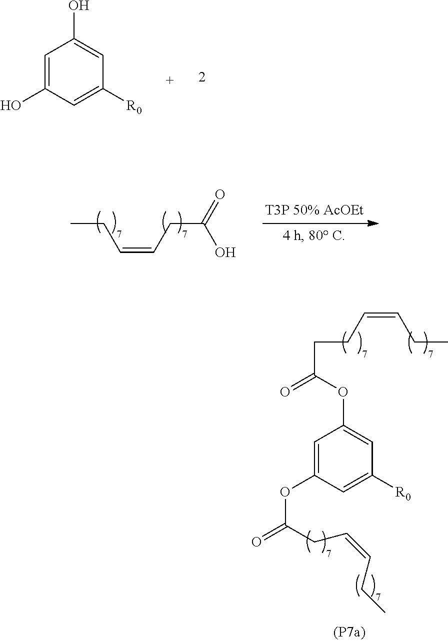

To a mixture of cardol (1 equivalent) and oleic acid (2 equivalents), were added 2 equivalents of propylphosphonic anhydride diluted to 50% in ethyl acetate at 80° C. The mixture was stirred for 4 hours. The reaction was monitored by thin-layer chromatography. At the end of the reaction, the solvent was evaporated under vacuum. The residue was dissolved in ethyl acetate and washed 3 times with water. The crude product P7a of formula obtained above is a colorless/yellowish oil. The yield is 97%.

¹H NMR (CDCl₃), δ (ppm): 6.78 (d, J=2.1 Hz, CH), 6.72 (d, J=2.1 Hz, CH), 5.82 (ddt, J=17.2, 10.1, 6.2 Hz, CH), 5.52-5.26 (m, CH), 5.14-4.92 (m, CH), 2.88-2.73 (m, CH₂), 2.63-2.55 (m, CH₂), 2.52 (t, J=7.5 Hz, CH₂), 2.16-1.95 (m, CH₂), 1.73 (p, J=7.5 Hz, CH₂), 1.60 (t, J=7.5 Hz, CH₂), 1.49-1.18 (m, CH₂), 1.03-0.77 (m, 2 CH₃).

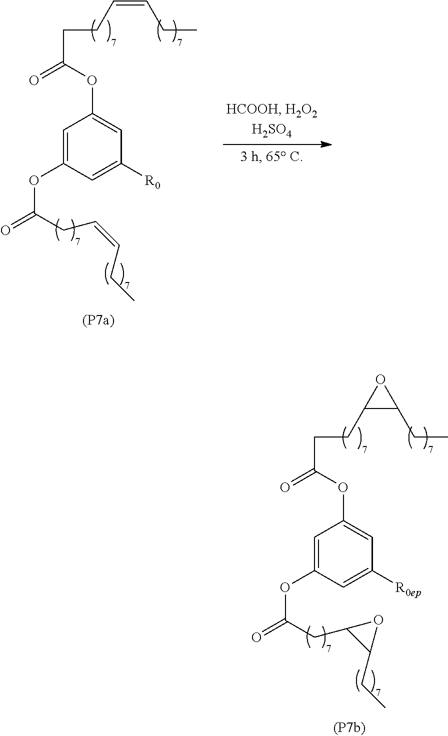

3 equivalents of P7a, formic acid (1 equivalent) and sulfuric acid (0.01 equivalent) were stirred and heated at 50° C. Hydrogen peroxide (6 equivalents) was added dropwise to the mixture, which was stirred and heated at 65° C. for 3 hours. At the end of the reaction, a liquid/liquid extraction step was performed using water and ethyl acetate. The organic phase was washed three times with water and then dried with MgSO₄. The solvent was then removed under vacuum. The crude product P7b of the above formula was purified by silica column chromatography using a 95:5 cyclohexane/ethyl acetate mixture. The P7b product obtained is a colorless oil. The yield was 94%.

¹H NMR (CDCl₃), δ (ppm): 6.78 (d, J=2.1 Hz, CH), 6.71 (t, J=2.1 Hz, CH), 5.99-4.96 (m, remaining alkenyl functions), 3.22-3.03 (m, CH), 3.03-2.95 (m, CH), 2.90 (dq, J=6.2, 3.4 Hz, CH₂), 2.65-2.55 (m, CH₂), 2.52 (dd, J=8.6, 6.4 Hz, CH₂), 2.45-2.20 (m, CH₂), 1.80-1.68 (m, CH₂), 1.67-1.20 (m, CH₂), 1.03-0.84 (m, 2 CH₃).

### Example 10 Esterification of Anacardic Acid

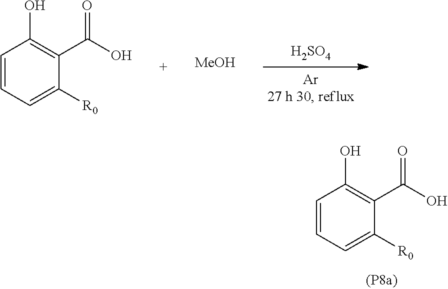

In a bicol flask with a dropping funnel and a 3 Å molecular sieve, a mixture of anacardic acid (1 equivalent) and dry methanol (15 mL per gram of cardanol) was dissolved in sulfuric acid (20 μL per 1 mL MeOH), stirred and heated under reflux and argon for 27.5 hours. At the end of the reaction, the solvent was evaporated under vacuum. The product was then solubilized in ethyl acetate. The organic phase was washed three times with brine and then dried with MgSO₄. The solvent was then removed under vacuum, and the residue dissolved in ethanol (96%) (10 mL ethanol per gram residue) and H₂O (500 μL per gram residue). Calcium hydroxide was introduced to precipitate the remaining anacardic acid, then the mixture was stirred for 20 minutes. After filtration, the filtrate was dried over MgSO₄ and the solvent evaporated under vacuum. The resulting product P8a of the above formula is a brown oil. The yield was 72%.

¹H NMR (CDCl₃), δ (ppm): 11.13 (s, 1H), 7.33-7.25 (dd, 2H), 6.84 (dd, J=8.3, 1.2 Hz, 1H), 6.72 (dd, J=7.5, 1.2 Hz, 1H), 5.88-5.75 (m, 1H), 5.49-5.26 (m, 4H), 5.09-4.92 (m, 1H), 3.96 (s, 3H), 2.93-2.85 (m, 2H), 2.85-2.74 (m, 3H), 2.12-1.95 (m, 4H), 1.66 (s, 2H), 1.50-1.56 (m, 3H), 1.44-1.16 (m, 17H), 0.89 (dt, J=13.1, 7.2 Hz, 3H).

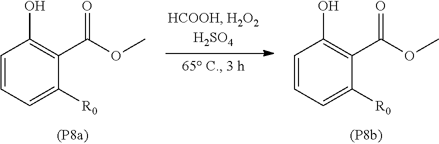

1 equivalent of P8a, formic acid (3 equivalents) and sulfuric acid (0.01 equivalents) were stirred and heated at 50° C. Hydrogen peroxide (2 equivalents) was added dropwise to the mixture, which was stirred and heated at 65° C. for 3 hours. After the reaction was complete, a liquid/liquid extraction step was carried out using water and ethyl acetate. The organic phase was washed three times with water and then dried with MgSO₄. The solvent was then removed under vacuum. The crude product P8b of the above formula was purified by silica column chromatography using a 95:5 cyclohexane/ethyl acetate mixture. The P8b product obtained is a colorless oil. The yield is 91%.

¹H NMR (CDCl₃), δ (ppm): 11.13 (s, 1H), 7.33-7.25 (dd, 2H), 6.84 (dd, J=8.3, 1.2 Hz, 1H), 6.72 (dd, J=7.5, 1.2 Hz, 1H), 5.88-5.75 (m, 1H), 5.49-5.26 (m, 4H), 5.09-4.92 (m, 1H), 3.96 (s, 3H), 2.93-2.85 (m, 2H), 2.85-2.74 (m, 3H), 2.12-1.95 (m, 4H), 1.66 (s, 2H), 1.50-1.56 (m, 3H), 1.44-1.16 (m, 17H), 0.89 (dt, J=13.1, 7.2 Hz, 3H)

### Example 11 Epoxidized Anacardic Acid

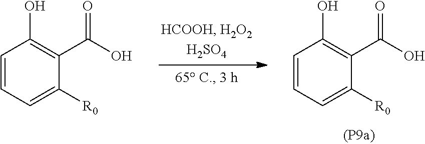

1 equivalent of anacardic acid, formic acid (3 equivalents) and sulfuric acid (0.01 equivalents) were stirred and heated at 50° C. Hydrogen peroxide (2 equivalents) was added dropwise to the mixture, which was stirred and heated at 65° C. for 3 hours. At the end of the reaction, a liquid/liquid extraction step was carried out using water and ethyl acetate. The organic phase was washed three times with water and then dried with MgSO₄. The solvent was then removed under vacuum. The crude product P9a of the above formula was purified by silica column chromatography using a 95:5 cyclohexane/ethyl acetate mixture. The P9a product obtained is a colorless oil. The yield was 91%.

¹H NMR (CDCl₃), δ (ppm): 11.13 (s, 1H), 7.33-7.25 (dd, CH), 6.85 (dd, J=8.3, 1.2 Hz, CH), 6.70 (dd, J=7.5, 1.2 Hz, CH), 5.94-4.98 (remaining alkenyl functions), 3.14-3.03 (CH₂), 3.01-2.6 (m, CH₂), 2.93-2.86 (m, CH₂), 2.86-2.75 (m, CH₂), 2.12-1.95 (m, CH₂), 1.72-1.66 (s, CH₂), 1.50-1.56 (m, CH₂), 1.41-1.15 (m, CH₂), 0.91-0.85 (m, J=13.1, 7.2 Hz, 1 CH₃)

### Example 12: Esterification of Anacardic Acid

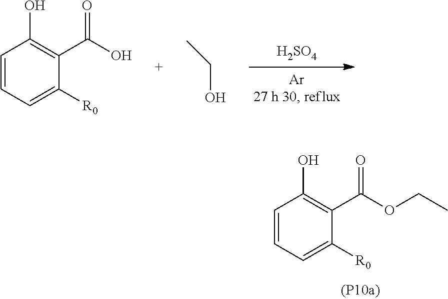

In a bicol flask with a dropping funnel and a 3 Å molecular sieve, a mixture of anacardic acid (1 equivalent) and dry ethanol (15 mL per gram of cardanol) was dissolved in sulfuric acid (20 μL per 1 mL EtOH)), then stirred and heated to reflux under argon for 27 h30. At the end of the reaction, the solvent was evaporated under vacuum. The product was then solubilized in ethyl acetate. The organic phase was washed three times with brine and then dried with MgSO₄. The solvent was then under vacuum, and the residue dissolved in ethanol (96%) (10 mL ethanol per gram residue) and H₂O (500 μL per gram residue). Calcium hydroxide was introduced to precipitate the remaining anacardic acid, then the mixture was stirred for 20 minutes. After filtration, the filtrate was dried over MgSO₄ and the solvent evaporated under vacuum. The resulting product P9a of the above formula is a brown oil. The yield was 56%.

¹H NMR (CDCl₃), δ (ppm): 7.29 (dd, J=8.3, 7.5 Hz, 1H), 6.84 (dd, J=8.3, 1.2 Hz, 1H), 6.72 (dd, J=7.6, 1.3 Hz, 1H), 5.89-5.74 (m, 1H), 5.50-5.28 (m, 5H), 5.11-4.93 (m, 1H), 4.44 (q, J=7.2 Hz, 1H), 2.94-2.87 (m, 1H), 2.86-2.74 (m, 3H), 2.11-1.93 (m, 5H), 1.60 (q, J=7.6 Hz, 4H), 1.44 (t, J=7.2 Hz, 1H), 1.41-1.19 (m, 22H), 0.94-0.82 (m, 4H).

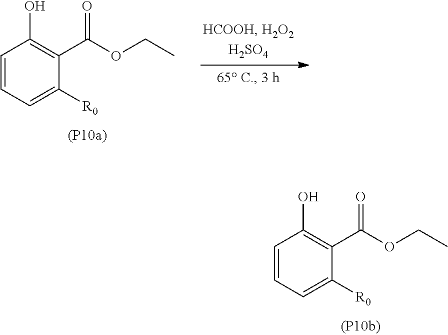

1 equivalents of P10a, formic acid (3 equivalents) and sulfuric acid (0.01 equivalents) were stirred and heated at 50° C. Hydrogen peroxide (2 equivalents) was added dropwise to the mixture, which was stirred and heated at 65° C. for 3 hours. At the end of the reaction, a liquid/liquid extraction step was carried out using water and ethyl acetate. The organic phase was washed three times with water and then dried with MgSO₄. The solvent was then removed under vacuum. The crude product P10b of the above formula was purified by silica column chromatography using a 95:5 cyclohexane/ethyl acetate mixture. The P10b product obtained is a colorless oil. The yield was 91%.

¹H NMR (CDCl₃), δ (ppm): 7.29 (dd, J=8.3, 7.5 Hz, 1H), 6.82 (dd, J=8.3, 1.2 Hz, 1H), 6.73 (dd, J=7.5, 1.2 Hz, 1H), 5.96-4.97 (remaining alkenyl functions), 4.45 (q, J=7.2 Hz, 1H), 3.15-3.04 (H), 3.01-2.94 (m, H), 2.92-2.85 (m, 2H), 2.85-2.76 (m, 3H), 2.12-1.95 (m, 5H), 1.59 (q, J=7.6 Hz, 4H), 1.45-1.14 (m, 20H), 0.91 (dt, J=13.1, 7.2 Hz, 3H)

### Example 13 Esterification of Anacardic Acid

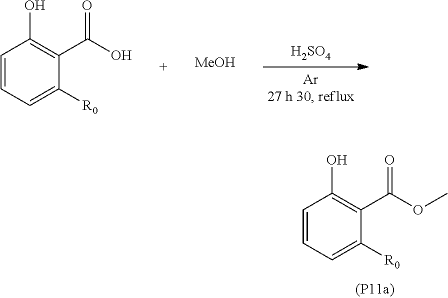

In a bicol flask with a dropping funnel and a 3 Å molecular sieve, a mixture of anacardic acid (1 equivalent) and dry methanol (15 mL per gram of cardanol) was dissolved in sulfuric acid (20 μL per 1 mL MeOH), placed under stirring and heated to reflux under argon for 27 h30. Once the reaction was complete, the solvent was evaporated under vacuum. The product was then solubilized in ethyl acetate. The organic phase was washed three times with brine and then dried with MgSO₄. The solvent was then removed under vacuum, and the residue dissolved in ethanol (96%) (10 mL ethanol per gram of residue) and H₂O (500 μL per gram of residue). Calcium hydroxide was introduced to precipitate the remaining anacardic acid, then the mixture was stirred for 20 minutes. After filtration, the filtrate was dried over MgSO₄ and the solvent evaporated under vacuum. The resulting product P11a of the above formula is a brown oil. The yield was 72%.

¹H NMR (CDCl₃), δ (ppm): 11.13 (s, 1H), 7.33-7.25 (dd, 2H), 6.84 (dd, J=8.3, 1.2 Hz, 1H), 6.72 (dd, J=7.5, 1.2 Hz, 1H), 5.88-5.75 (m, 1H), 5.49-5.26 (m, 4H), 5.09-4.92 (m, 1H), 3.96 (s, 3H), 2.93-2.85 (m, 2H), 2.85-2.74 (m, 3H), 2.12-1.95 (m, 4H), 1.66 (s, 2H), 1.50-1.56 (m, 3H), 1.44-1.16 (m, 17H), 0.89 (dt, J=13.1, 7.2 Hz, 3H).

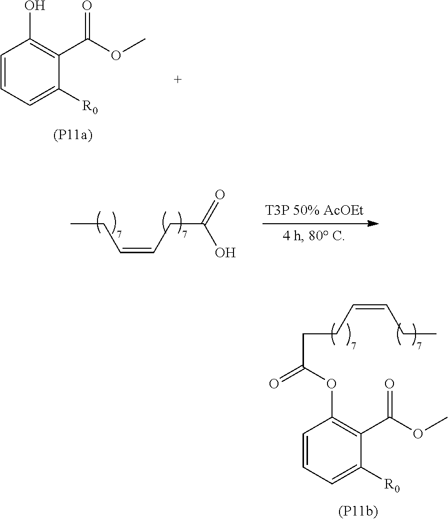

To a mixture of P11a (1 equivalent) and oleic acid (1 equivalent) was added 1 equivalent of propylphosphonic anhydride diluted to 50% in ethyl acetate at 80° C. The mixture was stirred for 4 hours. The reaction was monitored by thin-layer chromatography. At the end of the reaction, the solvent was evaporated under vacuum. The residue was dissolved in ethyl acetate and washed 3 times with water. The crude product P11b of the above formula is a colorless oil. The yield was 85%.

¹H NMR (CDCl₃), δ (ppm): 11.13 (s, 1H), 7.41-7.37 (dd, CH), 7.18-7.15 (dd, J=8.3, 1.2 Hz, CH), 7.01-6.98 (dd, J=7.5, 1.2 Hz, CH), 5.88-5.75 (m, CH), 5.49-5.26 (m, CH), 5.09-4.92 (m, CH), 3.96 (s, CH₃), 2.93-2.85 (m, CH₂), 2.85-2.74 (m, CH₂), 2.12-1.95 (m, CH₂), 1.72-1.66 (s, CH₂), 1.50-1.56 (m, CH₂), 1.44-1.16 (m, CH₂), 0.83-0.92 (m, J=13.1, 7.2 Hz, 2 CH₃).

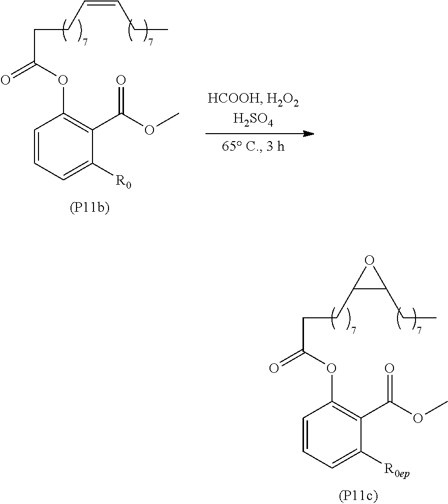

1 equivalent of P11b, formic acid (3 equivalents) and sulfuric acid (0.01 equivalents) were stirred and heated at 50° C. Hydrogen peroxide (2 equivalents) was added dropwise to the mixture, which was stirred and heated at 65° C. for 3 hours. At the end of the reaction, a liquid/liquid extraction step was carried out using water and ethyl acetate. The organic phase was washed three times with water and then dried with MgSO₄. The solvent was then removed under vacuum. The crude product P11c of the above formula was purified by silica column chromatography using a 95:5 cyclohexane/ethyl acetate mixture. The P11c product obtained is a colorless oil. The yield was 85%.

¹H NMR (CDCl₃), δ (ppm): 11.13 (s, 1H), 7.40-7.37 (dd, CH), 7.18-7.14 (dd, J=8.3, 1.2 Hz, CH), 7.00-6.98 (dd, J=7.5, 1.2 Hz, CH), 5.96-4.97 (remaining alkenyl functions), 3.96 (s, CH₃), 3.14-3.03 (CH₂), 3.02-2.6 (m, CH₂), 2.93-2.85 (m, CH₂), 2.85-2.74 (m, CH₂), 2.12-1.95 (m, CH₂), 1.72-1.66 (s, CH₂), 1.50-1.56 (m, CH₂), 1.44-1.16 (m, CH₂), 0.83-0.92 (m, J=13.1, 7.2 Hz, 2 CH₃)

### Example 14 Esterification of Anacardic Acid

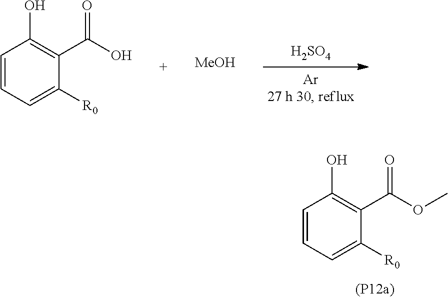

In a bicol flask with a dropping funnel and a 3 Å molecular sieve, a mixture of anacardic acid (1 equivalent) and dry methanol (15 mL per gram of cardanol) was dissolved in sulfuric acid (20 μL per 1 mL MeOH), placed under stirring and heated to reflux under argon for 27 h30. At the end of the reaction, the solvent was evaporated under vacuum. The product was then solubilized in ethyl acetate. The organic phase was washed three times with brine and then dried with MgSO₄. The solvent was then removed under vacuum, and the residue dissolved in ethanol (96%) (10 mL ethanol per gram of residue) and H₂O (500 μL per gram of residue). Calcium hydroxide was introduced to precipitate the remaining anacardic acid, then the mixture was stirred for 20 minutes. After filtration, the filtrate was dried over MgSO₄ and the solvent evaporated under vacuum. The resulting product P12a of the above formula is a brown oil. The yield was 72%.

¹H NMR (CDCl₃), δ (ppm): 11.13 (s, 1H), 7.33-7.25 (dd, 2H), 6.84 (dd, J=8.3, 1.2 Hz, 1H), 6.72 (dd, J=7.5, 1.2 Hz, 1H), 5.88-5.75 (m, 1H), 5.49-5.26 (m, 4H), 5.09-4.92 (m, 1H), 3.96 (s, 3H), 2.93-2.85 (m, 2H), 2.85-2.74 (m, 3H), 2.12-1.95 (m, 4H), 1.66 (s, 2H), 1.50-1.56 (m, 3H), 1.44-1.16 (m, 17H), 0.89 (dt, J=13.1, 7.2 Hz, 3H).

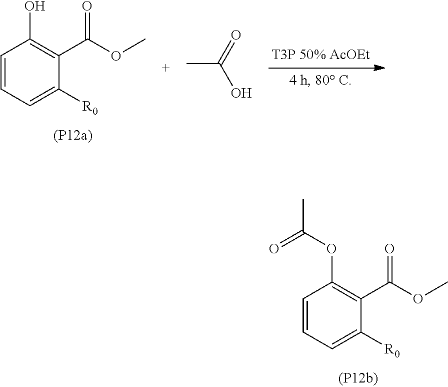

In a mixture of P12a (1 equivalent) and acetic acid (1 equivalent), 1 equivalent of propylphosphonic anhydride diluted to 50% in ethyl acetate was added at 80° C. The mixture was stirred for 4 hours. The reaction was monitored by thin-layer chromatography. At the end of the reaction, the solvent was evaporated under vacuum. The residue was dissolved in ethyl acetate and washed 3 times with water. The crude product P12b of the above formula is a colorless/yellowish oil. The yield was 89%.

¹H NMR (CDCl₃), δ (ppm): 11.13 (s, 1H), 7.40-7.37 (dd, CH), 7.18-7.15 (dd, J=8.3, 1.2 Hz, CH), 7.01-6.98 (dd, J=7.5, 1.2 Hz, CH), 5.88-5.75 (m, CH), 5.49-5.26 (m, CH), 5.09-4.92 (m, CH), 3.96 (s, CH₃), 2.93-2.85 (m, CH₂), 2.85-2.74 (m, CH₂), 2.30 (s, CH₃), 2.12-1.95 (m, CH₂), 1.72-1.66 (s, CH₂), 1.50-1.56 (m, CH₂), 1.44-1.16 (m, CH₂), 0.89-0.92 (m, J=13.1, 7.2 Hz, 1 CH₃).

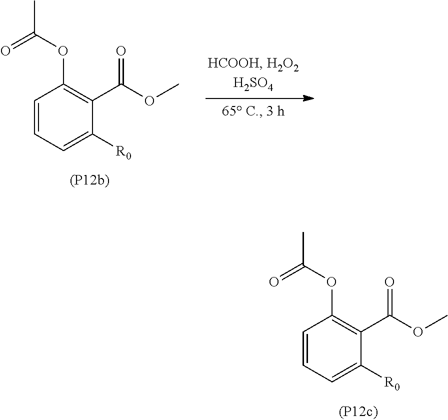

1 equivalent of P12b, formic acid (3 equivalents) and sulfuric acid (0.01 equivalents) were stirred and heated at 50° C. Hydrogen peroxide (2 equivalents) was added dropwise to the mixture, which was stirred and heated at 65° C. for 3 hours. At the end of the reaction, a liquid/liquid extraction step was carried out using water and ethyl acetate. The organic phase was washed three times with water and then dried with MgSO₄. The solvent was then removed under vacuum. The crude product P12c of the above formula was purified by silica column chromatography using a 95:5 cyclohexane/ethyl acetate mixture. The P12c product obtained is a colorless/yellowish oil. The yield is 87%.

¹H NMR (CDCl₃), δ (ppm): 11.13 (s, 1H), 7.41-7.38 (dd, CH), 7.18-7.16 (dd, J=8.3, 1.2 Hz, CH), 7.01-6.99 (dd, J=7.5, 1.2 Hz, CH), 5.96-4.97 (remaining alkenyl function), 3.96 (s, CH₃), 3.14-3.03 (CH₂), 3.02-2.6 (m, CH₂), 2.93-2.85 (m, CH₂), 2.85-2.74 (m, CH₂), 2.29 (s, CH₃), 2.12-1.95 (m, CH₂), 1.72-1.66 (s, CH₂), 1.50-1.56 (m, CH₂), 1.44-1.16 (m, CH₂), 0.91-0.88 (m, J=13.1, 7.2 Hz, 1 CH₃)

### Example 15: Extrusion Protocol

Cellulose acetate and plasticizer are mixed in an extruder. The plasticizer weight is at a concentration level of 25-32%. The extruder (twin screw (22)) is run at 150-190° C., at a rotation speed of 160 RPM.

### Example 16: Preparation of Plasticized Cellulose Acetate Solution and Plastic Film

The cellulose acetate used has a degree of substitution of 2.5.

A mixture of plasticizer (30% wt) and cellulose acetate was dissolved at 10% in acetone. The solutions were mixed for 24 hours at 25° C. under atmospheric pressure, then poured onto a Teflon or silicone tray. Films were made by slow evaporation of the solvent at 25° C. and at atmospheric pressure and were stored in a desiccator or oven.

### Thermogravimetric Analysis

The experiments consist in measuring the mass loss of the sample under air flow (60 mL·min⁻¹) as a function of temperature from 25 to 500° C. with a heating rate of 10° C.min⁻¹.

### Dynamic Mechanical Analysis

The samples were tested under uniaxial tension during heating at a rate of 3° C./min at a frequency of 1 Hz with a fixed stress of 2.1·10⁻⁶ m for samples plasticized with diethylphthalate and 3.6·10⁻⁶ m for samples plasticized with the plasticizers of the invention.

The samples tested were 0.1 mm thick, 10 mm wide and 20 mm long.

### Example 17 Plastic Film with P1a

The plastic film was prepared with plasticizer P1a according to Example 14. The results were as follows.

Differential scanning calorimetry showed a glass transition temperature of 192° C.

### Example 18 Plastic Film with P1b

The plastic film was prepared with plasticizer P1b according to Example 14. The results were as follows.

The glass transition temperature obtained by mechanical dynamic analysis was 128° C. Differential scanning calorimetry showed a glass transition temperature of 171° C. Thermogravimetric analysis showed that there was no loss of thermal stability for the plastic.

The temperature at which 5% of the film mass is lost is 311° C. The onset temperature for film degradation is 350° C.

The degradation temperature of the entire plastic film is 371° C.

### Example 19 Plastic Film with P5

The plastic film was prepared with plasticizer P5 according to example 14. The results were as follows.

Differential scanning calorimetry showed a glass transition temperature of 178° C.

### Example 20 Plastic Film with Cardol

Preparation of the plastic film with cardol was carried out according to Example 14. The results were as follows.

Dynamic mechanical analysis showed a glass transition temperature of 114° C.

The temperature at which 5% of the film mass is lost is 311° C. The onset temperature for film degradation is 409° C.

The degradation temperature of the entire plastic film is 425° C.

### Example 21 Plastic Film with P6a

Preparation of the plastic film with plasticizer P6a was carried out according to Example 14. The results were as follows.

Dynamic mechanical analysis showed a glass transition temperature of 183° C.

### Example 22 Plastic Film with P6c

The plastic film was prepared with the plasticizer Cardol C2 diol according to Example 14. The results were as follows.

Dynamic mechanical analysis showed a glass transition temperature of 165° C.

The degradation temperature of 5% of the film is 247° C.

The degradation temperature of the entire plastic film is 430° C.

### Example 23 Plastic Film with P2a

Preparation of the plastic film with plasticizer P2a was carried out according to Example 14. The results were as follows.

Dynamic mechanical analysis showed a glass transition temperature of 171° C.

The temperature at which 5% of the film mass is lost is 244° C. The onset temperature for film degradation is 344° C.

The degradation temperature of the entire plastic film is 378° C.

### Example 24 Plastic Film with P2b

Preparation of the plastic film with plasticizer P2b was carried out according to Example 14. The results were as follows.

Dynamic mechanical analysis showed a glass transition temperature of 191° C.

### Example 25 Plastic Film with P2c

Preparation of the plastic film with the plasticizer Cardanol C2 diols was carried out according to Example 14. The results were as follows.

Dynamic mechanical analysis showed a glass transition temperature of 164° C.

1.-25. (canceled)

26. A process for preparing a plastic material comprising or consisting of cellulose acetate, comprising a step of mixing cellulose acetate with a composition comprising
1) at least one primary plasticizer of cellulose acetate, in an extruder, said composition comprising as primary plasticizer at least one compound derived from CNSL which has at least one ester function and/or at least one epoxide function or at least one compound of formula (1), or
2) at least one primary plasticizer of cellulose acetate, in a solvent, to form a solution, said composition comprising as primary plasticizer at least one compound derived from CNSL which has at least one ester function and/or at least one epoxide function or at least one compound of formula (1), 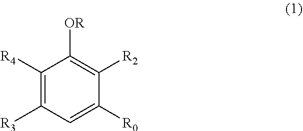
formula (1) in which
R is selected from:
a hydrogen atom or
a CO—R1 group,
R₀ is selected from:
a linear alkyl group of 15 carbon atoms, optionally comprising from 1 to 3 epoxide functions or,
a linear alkenyl group of 15 carbon atoms, comprising x alkenyl functions, x varying from 1 to 3, comprising y epoxide functions, y varying from 0 to 2, x+y being less than or equal to 3,
R₂ is selected from:
a hydrogen atom,
a COOH carboxylic acid function,
CO—O—R₅ with formula 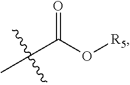
R₃ is selected from:
a hydrogen atom,
an alcohol function —OH
OC—O—R₆ with formula 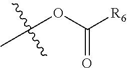
R₄ is selected from:
a hydrogen atom or
a methyl group CH₃;
R₁, R₅ and R₆ are identical or different and are selected from:
a linear alkyl group of 1 to 30 carbon atoms, optionally containing 1 to 6 epoxide functions, or
a linear alkenyl group of 2 to 24 carbon atoms, optionally comprising 1 to 6 epoxide functions, said alkenyl group being optionally substituted by an alkyl group of 1 to 5 carbon atoms,
R₀, R₁, R₅ and R₆ optionally comprising one to six substituent(s) selected from —OH, —OR₇ or OCOR₇, wherein R₇ is an alkyl group of 1 to 3 carbon atoms,
with the proviso that at least one of the OR, R₂ or R₃ groups is an ester function and/or at least one of R₀, R₁, R₅ or R₆ comprises at least one epoxide function.
or at least one of the compounds of the following formulae: 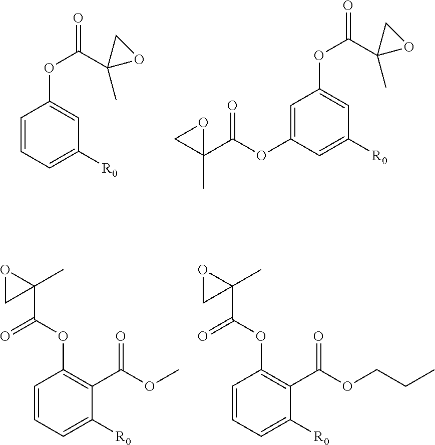
to obtain said plastic material.

27. The process, according to claim 26, comprising a step of evaporating said solvent from said solution, to obtain said plastic material.

28. The process, according to claim 26, wherein the total amount by weight of cellulose acetate and primary plasticizer is from 1 to 50% of the solution by weight, in particular from 10 to 30, preferably 10%.

30. The process, according to claim 26, wherein the ratio between the amount of primary plasticizer by weight and cellulose acetate by weight ranges from 1 to 50% by weight, in particular from 10 to 30%, preferably 30%.

31. The process, according to claim 26, wherein, in the composition comprising the primary plasticizer, the latter is a compound of formula (1) 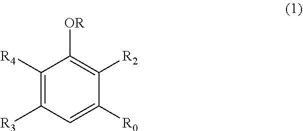
formula (1) in which
R is selected from:
a hydrogen atom or
a CO—R1 group,
R₀ is selected from:
a linear alkyl group of 15 carbon atoms, optionally comprising from 1 to 3 epoxide functions or,
a linear alkenyl group of 15 carbon atoms, comprising x alkenyl functions, x varying from 1 to 3, comprising y epoxide functions, y varying from 0 to 2, x+y being less than or equal to 3,
R₂ is selected from:
a hydrogen atom,
a COOH carboxylic acid function,
CO—O—R₅ of formula 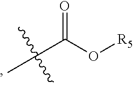
R₃ is selected from:
a hydrogen atom,
an alcohol function —OH
OC—O—R₆ with formula 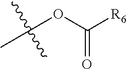
R₄ is selected from:
a hydrogen atom or
a methyl group CH₃;
R₁, R₅ and R₆ are identical or different and are selected from:
a linear alkyl group of 1 to 30 carbon atoms, optionally containing 1 to 6 epoxide functions, or
a linear alkenyl group of 2 to 24 carbon atoms, optionally comprising 1 to 6 epoxide functions, said alkenyl group being optionally substituted by an alkyl group of 1 to 5 carbon atoms,
R₀ optionally comprising one to six substituent(s) selected from —OH, —OR₇ or OCOR₇, wherein R₇ is an alkyl group of 1 to 3 carbon atoms,
R₁, R₅ and R₆ optionally comprising one or two substituents selected from —OH, —OR₇ or OCOR₇, with the proviso that at least one of the OR, R₂ or R₃ groups is an ester function and/or at least one of
R₀, R₁, R₅ or R₆ comprises at least one epoxide function.
or at least one of the compounds of the following formulae:

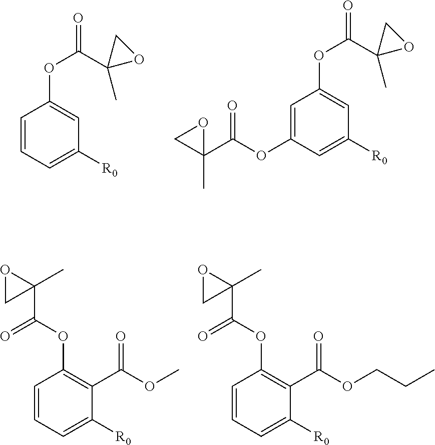

32. The process, according to claim 26, wherein, in the composition comprising the primary plasticizer, the latter is a compound of formula (1) 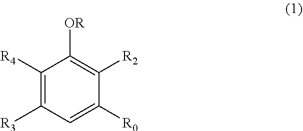
formula (1) in which
R is selected from:
a hydrogen atom or
a CO—R1 group,
R₀ is selected from:
a linear alkyl group of 15 carbon atoms, optionally comprising from 1 to 3 epoxide functions or,
a linear alkenyl group of 15 carbon atoms, comprising x alkenyl functions, x varying from 1 to 3, comprising y epoxide functions, y varying from 0 to 2, x+y being less than or equal to 3,
R₂ is selected from:
a hydrogen atom,
a COOH carboxylic acid function,
CO—O—R₅ of formula 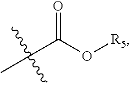
R₃ is selected from:
a hydrogen atom,
an alcohol function —OH
OC—O—R₆ with formula 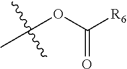
R₄ is selected from:
a hydrogen atom or
a methyl group CH₃;
R₁, R₅ and R₆ are identical or different and are selected from:
a linear alkyl group of 1 to 30 carbon atoms, optionally containing 1 to 6 epoxide functions, or
a linear alkenyl group of 2 to 24 carbon atoms, optionally comprising 1 to 6 epoxide functions, said alkenyl group being optionally substituted by an alkyl group of 1 to 5 carbon atoms,
R₁, R₅ and R₆ optionally comprising one or two substituents selected from —OH, —OR₇ or OCOR₇, wherein R₇ is an alkyl group of 1 to 3 carbon atoms,
with the proviso that at least one of the OR, R₂ or R₃ groups is an ester function and/or at least one of R₀, R₁, R₅ or R₆ comprises at least one epoxide function.
or at least one of the compounds of the following formulae:

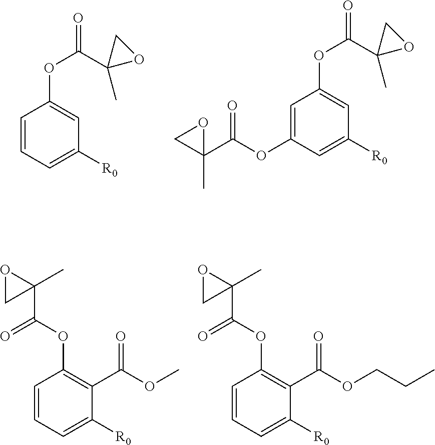

33. The process, according to claim 26, wherein, in the composition comprising the primary plasticizer, the latter is at least one compound of formula (1b) 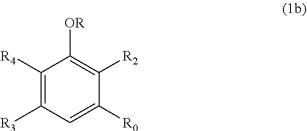
formula (1b) in which
R is selected from:
a hydrogen atom
a CO—R₁ group,
R₀ is selected from:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3.
R₂ is selected from:
a hydrogen atom,
a COOH carboxylic acid function, or
CO—O—R₅ with formula 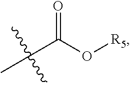
R₃ is selected from:
a hydrogen atom,
an OH alcohol function, or
OC—O—R₆ with formula 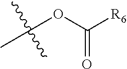
R₄ is selected from:
a hydrogen atom,
a methyl group CH₃,
R₁, R₅ and R₆ are identical or different and are selected from:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
R₀ optionally comprising one to six substituent(s) selected from —OH, —OR₇ or OCOR₇, wherein R₇ is an alkyl group of 1 to 3 carbon atoms,
R₁, R₅ and R₆ optionally comprising one to six substituent(s), in particular one or two, selected from —OH, —OR₇ or OCOR₇,
with the proviso that at least one of the OR, R₂ or R₃ groups is an ester function;
or at least one compound of formula (1c), 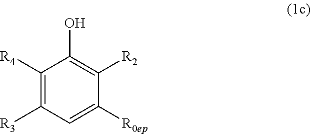
formula (1c) in which
R₀ₑₚ is selected from:
a linear alkyl group of 15 carbon atoms containing from 1 to 3 epoxide functions, or
a linear alkenyl group of 15 carbon atoms, comprising x alkenyl functions, x varying from 1 to 2, comprising y epoxide functions, y varying from 1 to 2, x+y being less than or equal to 3,
R₀ₑₚ optionally comprising one to six substituent(s) selected from —OH, OR₇ or OCOR₇, wherein R₇ is an alkyl group of 1 to 3 carbon atoms
R₂ is selected from:
a hydrogen atom,
a COOH carboxylic acid function,
R₃ is selected from:
a hydrogen atom,
an OH alcohol function,
R₄ is selected from:
a hydrogen atom,
a methyl group CH₃;
or at least one compound of formula (1d), 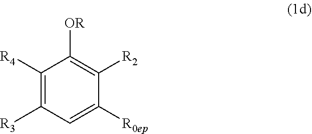
formula (1d) in which:
R is selected from:
a hydrogen atom
a CO—R₁ group,
R₀ₑₚ is selected from:
a linear alkyl group of 15 carbon atoms optionally containing 1 to 3 epoxide functions, or
a linear alkenyl group of 15 carbon atoms, comprising x alkenyl functions, x varying from 1 to 3, optionally comprising y epoxide functions, y varying from 0 to 2, x+y being less than or equal to 3,
R₂ is selected from:
a hydrogen atom,
a COOH carboxylic acid function, or
CO—O—R₅ with formula 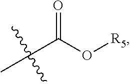
R₃ is selected from
a hydrogen atom,
an OH alcohol function, or
OC—O—R₆ formula 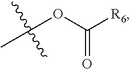
R₄ is selected from:
a hydrogen atom,
a methyl group CH₃,
R₁, R₅ and R₆ are identical or different and are selected from:
a linear alkyl group of 1 to 30 carbon atom(s), optionally containing 1 to 6 epoxide function(s), or
a linear alkenyl group of 2 to 24 carbon atoms, optionally comprising 1 to 6 epoxide function(s), said alkenyl group being optionally substituted by an alkyl group of 1 to 5 carbon atom(s),
R₀ₑₚ optionally comprising one to six substituent(s) selected from —OH, —OR₇ or OCOR₇, wherein R₇ is an alkyl group of 1 to 3 carbon atoms,
R₁, R₅ and R₆ optionally comprising one to six substituent(s), in particular one or two, selected from —OH, —OR₇ or OCOR₇,
with the proviso that at least one of the OR, R₂ or R₃ groups is an ester function and at least one of the R₀ₑₚ, R₁, R₅ or R₆ groups comprises at least one epoxide function.

34. The process, according to claim 26, wherein, the compound is of formula (Tb) and is selected from compounds of the following formulae (2), (3), (4), (5) and (6), 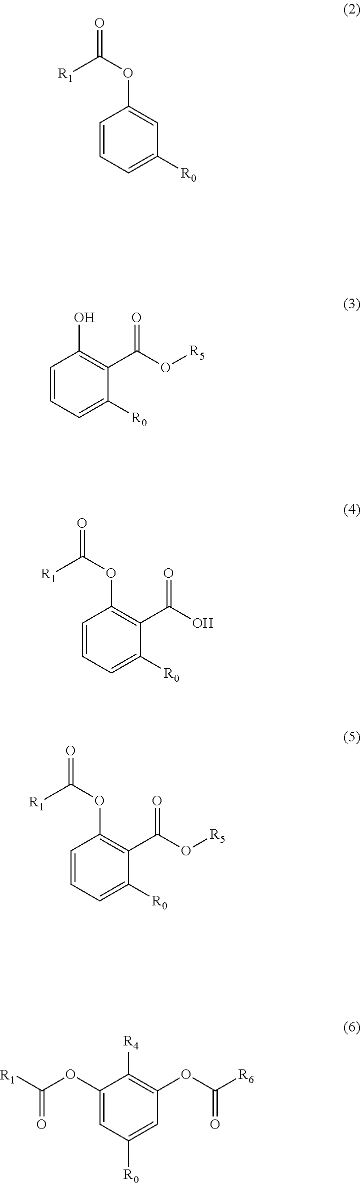
in which R₀ is selected from:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
R₄ is selected from:
a hydrogen atom,
a methyl group CH₃
R₁, R₅ and R₆ are selected from:
a linear alkyl group of 1 to 30 carbon atom(s), or
a linear alkenyl group of 2 to 24 carbon atoms, said alkenyl group being optionally substituted by an alkyl group of 1 to 5 carbon atoms,
said R₀ group optionally comprising one to six substituent(s) selected from —OH, —OR₇ or OCOR₇, wherein R₇ is an alkyl group of 1 to 3 carbon atom(s),
said R₁, R₅ and R₆ groups optionally comprising one to six substituent(s), in particular one or two, selected from —OH, —OR₇ or OCOR₇,
or in which the compound is of formula (1c) and is selected from compounds of the following formulae (7), (8) and (9), 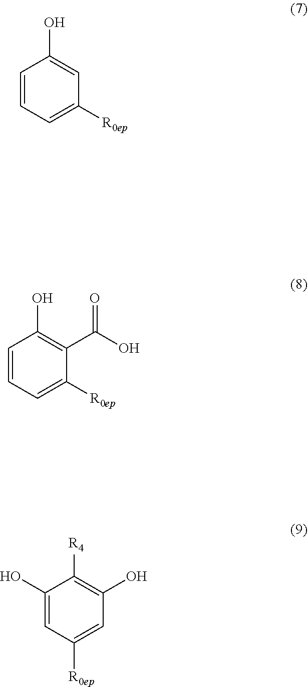
in which R₀ₑₚ is selected from:
a linear alkyl group of 15 carbon atoms containing from 1 to 3 epoxide functions, or
a linear alkenyl group of 15 carbon atoms, comprising x alkenyl functions, x varying from 1 to 2, comprising y epoxide functions, y varying from 1 to 2, x+y being less than or equal to 3;
R₀ₑₚ optionally comprising one to six substituent(s) selected from —OH, OR₇ or OCOR₇, wherein
R₇ is an alkyl group of 1 to 3 carbon atoms
or in which the compound is of formula (1d) and is selected from compounds of the following formulae (10), (11), (12), (13) and (14): 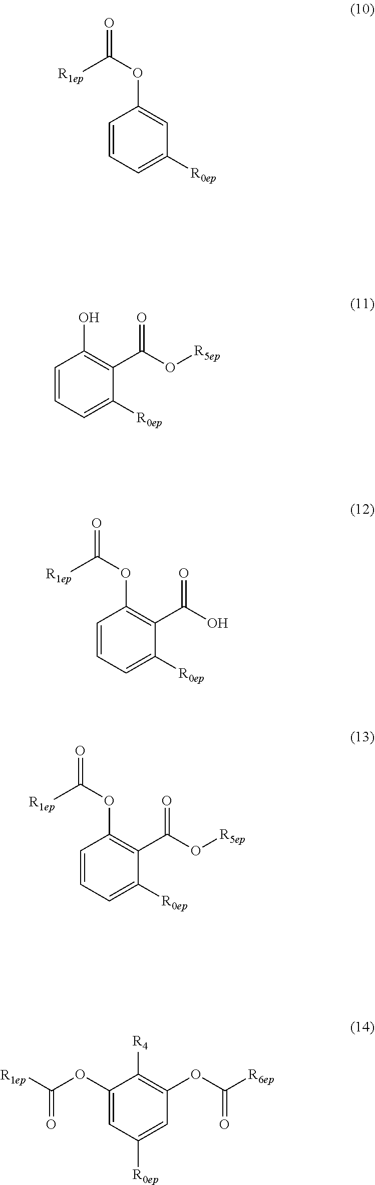
in which R₀ₑₚ is selected from:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
R₄ is selected from:
a hydrogen atom, or
a methyl group CH₃,
R₁ₑₚ, R₅ₑₚ and R₆ₑₚ are selected from:
a linear alkyl group of 1 to 30 carbon atom(s), optionally containing 1 to 6 epoxide function(s), or
a linear alkenyl group of 2 to 24 carbon atoms, optionally comprising 1 to 6 epoxide function(s), said alkenyl group being optionally substituted by an alkyl group of 1 to 5 carbon atom(s),
said R₀ₑₚ group optionally comprising one to six substituent(s) selected from —OH, —OR₇ or OCOR₇, wherein R₇ is an alkyl group of 1 to 3 carbon atom(s),
said R₁ₑₚ, R₅ₑₚ and R₆ₑₚ groups optionally comprising one to six substituent(s), in particular one or two, selected from —OH, —OR₇ or OCOR₇,
with the proviso that at least one of the R₀ₑₚ, R₁ₑₚ, R₅ₑₚ or R₆ₑₚ groups contains at least one epoxide function.

35. The process, according to claim 26, wherein, in the composition comprising the primary plasticizer, the latter is a compound of formula (2) 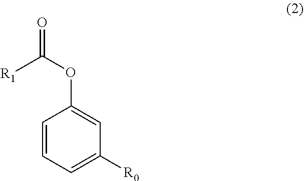
formula (2) in which R₀ represents:
a linear alkyl group of 15 carbon atoms optionally containing from 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3.
formula (2) in which R₁ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
R₁ optionally comprising one to six substituent(s), in particular one or two, selected from —OH, —OR₇ or OCOR₇, wherein R₇ is an alkyl group of 1 to 3 carbon atoms
R₀ optionally comprising one to six substituent(s) selected from —OH, —OR₇ or OCOR₇
and further comprising a step A of preparing said compound of formula (2) from a compound of formula (15) with an acid of formula (16): 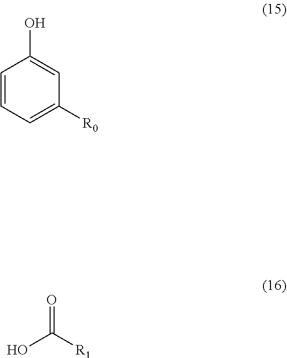
formula (15) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
formula (16) in which R₁ represents:
linear alkyl group of 1 to 30 carbon atom(s), or
a linear alkenyl group of 2 to 24 carbon atom(s), comprising 1 to 6 alkenyl function(s), said alkenyl group being optionally substituted by a linear alkyl group of 1 to 5 carbon atom(s),
R₁ optionally comprising one to six substituent(s), in particular one or two, selected from —OH, —OR₇ or OCOR₇, wherein R₇ is an alkyl group of 1 to 3 carbon atoms
R₀ optionally comprising one to six substituent(s) selected from —OH, —OR₇ or OCOR₇.

36. The process, according to claim 26, wherein, in the composition comprising the primary plasticizer, the latter is a compound of formula (3) 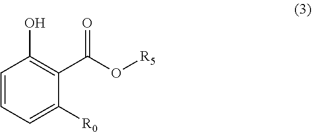
formula (3) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3, formula (3) in which R₅ represents:
a linear alkyl group of 1 to 30 carbon atom(s), or
a linear alkenyl group of 2 to 24 carbon atoms, comprising 1 to 6 alkenyl functions, optionally branched by a linear alkyl group of 1 to 5 carbon atoms,
R₅ optionally comprising one to six substituent(s), in particular one or two, selected from —OH, —OR₇ or OCOR₇, wherein R₇ is an alkyl group of 1 to 3 carbon atoms
R₀ optionally comprising one to six substituent(s) selected from —OH, —OR₇ or OCOR₇, and further comprising a step B of preparing a compound of formula (3) from a compound of formula (17) and an alcohol of formula (18) 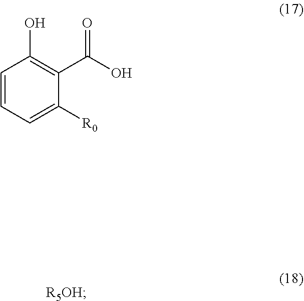
formula (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
formula (18) in which R₅ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
R₅, in addition to the —OH group represented in formula (18), optionally comprising one to six substituent(s), in particular one or two, selected from —OH, —OR₇ or OCOR₇, wherein R₇ is an alkyl group of 1 to 3 carbon atoms
R₀ optionally comprising one to six substituent(s) selected from —OH, —OR₇ or OCOR₇,
or in which, in the composition comprising the primary plasticizer, the primary plasticizer is a compound of formula (4) 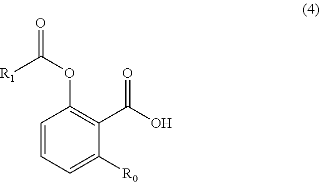
formula (4) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
formula (4) in which R₁ represents:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
R₁ optionally comprising one to six substituent(s), in particular one or two, selected from —OH, —OR₇ or OCOR₇, wherein R₇ is an alkyl group of 1 to 3 carbon atoms
R₀ optionally comprising one to six substituent(s) selected from —OH, —OR₇ or OCOR₇,
and further comprising a step C of preparing a compound of formula (4) from a compound of formula (17) and an acid of formula (19): 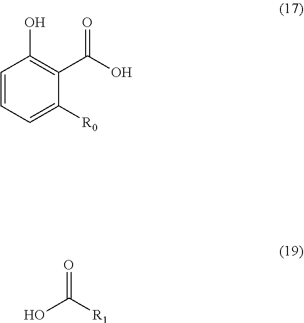
formula (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
formula (19) in which R₁ represents:
a linear alkyl group of 1 to 30 carbon atoms, or
a linear alkenyl group of 2 to 24 carbon atoms, comprising 1 to 6 alkenyl function(s), said alkenyl group being optionally substituted by a linear alkyl group of 1 to 5 carbon atom(s),
said R₁ group optionally comprising one to six substituent(s), in particular one or two, selected from —OH, —OR₇ or OCOR₇, wherein R₇ is an alkyl group of 1 to 3 carbon atoms
said R₀ group optionally comprising one to six substituent(s) selected from —OH, —OR₇ or OCOR₇;
or in which, in the composition comprising the primary plasticizer, the primary plasticizer is a compound of formula (5) 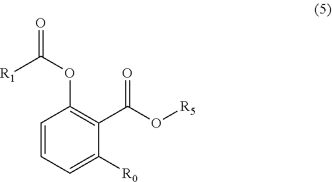
formula (5) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
R₁ and R₅ represent:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
R₁ and R₅ optionally comprising one to six substituent(s), in particular one or two, selected from —OH, —OR₇ or OCOR₇, wherein R₇ is an alkyl group of 1 to 3 carbon atoms
R₀ optionally comprising one to six substituent(s) selected from —OH, —OR₇ or OCOR₇,
and further comprising a step D of preparing a compound of formula (5) from a compound of formula (17) with an alcohol of formula (18) and an acid of formula (19): 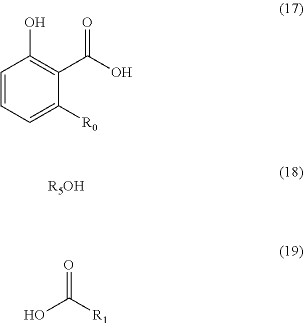
formula (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3.
formulae (18) and (19) in which R₁ and R₅ independently represent:
a linear alkyl group of 1 to 30 carbon atoms, or
a linear alkenyl group of 2 to 24 carbon atoms, comprising 1 to 6 alkenyl function(s), said alkenyl group being optionally substituted by a linear alkyl group of 1 to 5 carbon atom(s),
said R₁ groups optionally comprising one to six substituent(s), in particular one or two, selected from —OH, —OR₇ or OCOR₇, wherein R₇ is an alkyl group of 1 to 3 carbon atoms
R₅, in addition to the —OH group represented in formula (18), optionally comprising one to six substituent(s), in particular one or two, selected from —OH, —OR₇ or OCOR₇
said R₀ group optionally comprising one to six substituent(s) selected from —OH, —OR₇ or OCOR₇.

37. The process, according to claim 26, wherein, in the composition comprising the primary plasticizer, the latter is a compound of formula (6) 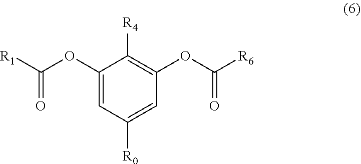
formula (6) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3
R₄ represents:
a hydrogen atom,
a methyl group CH₃,
R₁ and R₆ independently represent, in particular may be identical:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
R₁ and R₆ optionally comprising one to six substituent(s), in particular one or two, selected from —OH, —OR₇ or OCOR₇, wherein R₇ is an alkyl group of 1 to 3 carbon atoms
R₀ optionally comprising one to six substituent(s) selected from —OH, —OR₇ or OCOR₇,
and further comprising a step E of preparing said compound of formula (6) from a compound of formula (20) with an acid of formula (21) and an acid of formula (22) 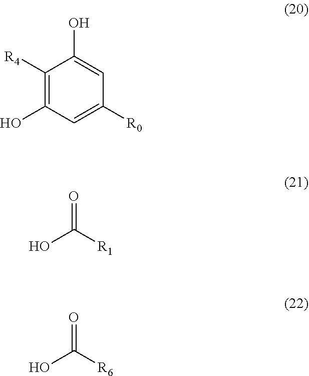
formula (20) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3.
R₄ represents:
a hydrogen atom,
a methyl group CH₃,
formulae (21) and (22) in which R₁ and R₆ independently represent, in particular may be identical:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
R₁ and R₆ optionally comprising one to six substituent(s), in particular one or two, selected from —OH, —OR₇ or OCOR₇, wherein R₇ is an alkyl group of 1 to 3 carbon atoms
R₀ optionally comprising one to six substituent(s) selected from —OH, —OR₇ or OCOR₇.

38. The process, according to claim 26, in which, in the composition comprising the primary plasticizer, the latter is a compound of formula (7) 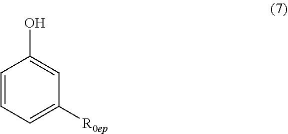
formula (7) in which R₀ₑₚ represents:
a linear alkyl group of 15 carbon atoms containing from 1 to 3 epoxide functions, or
a linear alkenyl group of 15 carbon atoms, comprising x alkenyl functions, x varying from 1 to 2, comprising y epoxide functions, y varying from 1 to 2, x+y being less than or equal to 3,
R₀ₑₚ optionally comprising one to six substituent(s) selected from —OH, —OR₇ or OCOR₇, wherein R₇ is an alkyl group of 1 to 3 carbon atoms
and further comprising a step of preparing F said compound of formula (7) from a compound of formula (15) 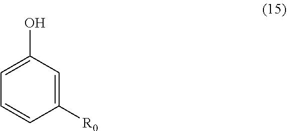
formula (15) in which R₀ represents:
a linear alkenyl group of 15 carbon atoms, comprising x alkenyl functions, x varying from 1 to 3, comprising y epoxide functions, y varying from 0 to 2, x+y being less than or equal to 3,
R₀ optionally comprising one to six substituent(s) selected from —OH, —OR₇ or OCOR₇, wherein R₇ is an alkyl group of 1 to 3 carbon atoms,
or in which, in the composition comprising the primary plasticizer, the primary plasticizer is a compound of formula (8) 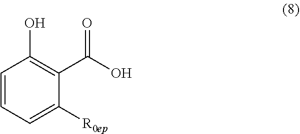
formula (8) in which R₀ₑₚ represents:
a linear alkyl group of 15 carbon atoms containing from 1 to 3 epoxide functions, or
a linear alkenyl group of 15 carbon atoms, comprising x alkenyl functions, x varying from 1 to 2, comprising y epoxide functions, y varying from 1 to 2, x+y being less than or equal to 3,
R₀ₑₚ optionally comprising one to six substituent(s) selected from —OH, —OR₇ or OCOR₇, wherein R₇ is an alkyl group of 1 to 3 carbon atoms
and further comprising a step of preparing G a compound of formula (8) from a compound of formula (17) 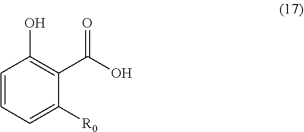
formula (17) in which R₀ represents:
a linear alkenyl group of 15 carbon atoms, comprising x alkenyl functions, x varying from 1 to 3, comprising y epoxide functions, y varying from 0 to 2, x+y being less than or equal to 3,
R₀ optionally comprising one to six substituent(s) selected from —OH, —OR₇ or OCOR₇, wherein R₇ is an alkyl group of 1 to 3 carbon atoms;
or in which, in the composition comprising the primary plasticizer, the primary plasticizer is a compound of formula (9) 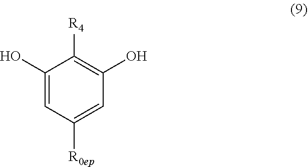
formula (9) in which R₀ₑₚ represents:
a linear alkyl group of 15 carbon atoms containing from 1 to 3 epoxide functions, or
a linear alkenyl group of 15 carbon atoms, comprising x alkenyl functions, x varying from 1 to 2, comprising y epoxide functions, y varying from 1 to 2, x+y being less than or equal to 3,
R₀ₑₚ optionally comprising one to six substituent(s) selected from —OH, —OR₇ or OCOR₇, wherein R₇ is an alkyl group of 1 to 3 carbon atoms
R₄ represents:
a hydrogen atom,
a methyl group CH₃,
and further comprising a step of preparing H said compound of formula (9) from a compound of formula (20) 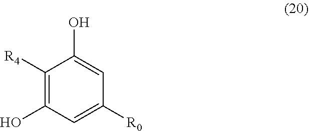
formula (20) in which R₀ represents
a linear alkenyl group of 15 carbon atoms, comprising x alkenyl functions, x varying from 1 to 3, comprising y epoxide functions, y varying from 0 to 2, x+y being less than or equal to 3,
R₀ optionally comprising one to six substituent(s) selected from —OH, —OR₇ or OCOR₇, wherein R₇ is an alkyl group of 1 to 3 carbon atoms
R₄ represents:
a hydrogen atom,
a methyl group CH₃.

39. The process, according to claim 26, wherein, in the composition comprising the primary plasticizer, the latter is a compound of formula (10) 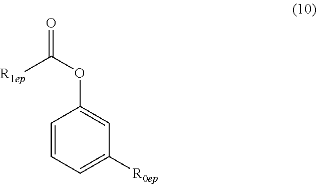
formula (10) in which R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
R₀ₑₚ optionally comprising one to six substituent(s) selected from —OH, —OR₇ or OCOR₇, wherein R₇ is an alkyl group of 1 to 3 carbon atoms,
R₁ₑₚ represents:
a linear alkyl group of 1 to 30 carbon atom(s), optionally containing 1 to 6 epoxide function(s), or
a linear alkenyl group of 2 to 24 carbon atoms, optionally comprising 1 to 6 epoxide function(s), said alkenyl group being optionally substituted by an alkyl group of 1 to 5 carbon atom(s),
R₁ₑₚ optionally comprising one to six substituent(s) selected from —OH, —OR₇ or OCOR₇, wherein R₇ is an alkyl group of 1 to 3 carbon atoms,
with the proviso that at least one of the R₀ₑₚ or R₁ₑₚ groups contains at least one epoxide function,
and further comprising a step A of preparing a compound of formula (2) from a compound of formula (15) with an acid of formula (16) 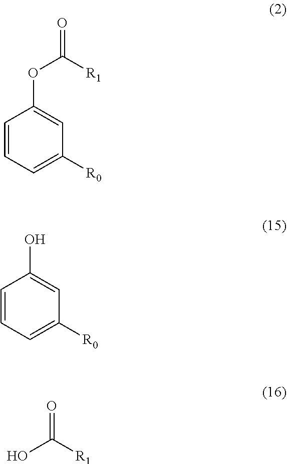
formulae (2) and (15) in which R₀ represent:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3
R₀ optionally comprising one to six substituent(s) selected from —OH, —OR₇ or OCOR₇, wherein R₇ is an alkyl group of 1 to 3 carbon atoms
formulae (2) and (16) in which R₁ represent:
a linear alkyl group of 1 to 30 carbon atoms, or
a linear alkenyl group of 2 to 24 carbon atoms, comprising 1 to 6 alkenyl functions, said alkenyl group being optionally substituted by a linear alkyl group of 1 to 5 carbon atoms,
R₁ optionally comprising one to six substituent(s) selected from —OH, —OR₇ or OCOR₇, wherein R₇ is an alkyl group of 1 to 3 carbon atoms,
and further comprising a step I of preparing the compound of formula (10) from a compound of formula (2),
with the proviso that at least one of the R₀ and R₁ groups contains an alkenyl function.

40. The process, according to claim 26, wherein, in the composition comprising the primary plasticizer, this is a compound of formula (11), 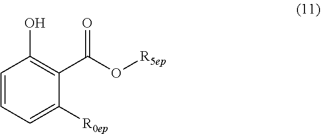
formula (11) in which R₀ₑₚ represents:
a linear alkyl group of 15 carbon atoms optionally containing from 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
R₀ₑₚ optionally comprising one to six substituent(s) selected from —OH, —OR₇ or OCOR₇, wherein R₇ is an alkyl group of 1 to 3 carbon atoms
R₅ₑₚ represents:
a linear alkyl group of 1 to 30 carbon atom(s), optionally containing 1 to 6 epoxide function(s), or
a linear alkenyl group of 2 to 24 carbon atoms, optionally comprising 1 to 6 epoxide function(s), said alkenyl group being optionally substituted by an alkyl group of 1 to 5 carbon atom(s),
R₅ₑₚ optionally comprising one to six substituents, in particular one or two, selected from —OH, —OR₇ or OCOR₇, wherein R₇ is an alkyl group of 1 to 3 carbon atoms,
with the proviso that at least one of the R₀ₑₚ or R₅ₑₚ groups contains at least one epoxide function and further comprising a preparation step B of a compound of formula (3) from a compound of formula (17) and an alcohol of formula (18) 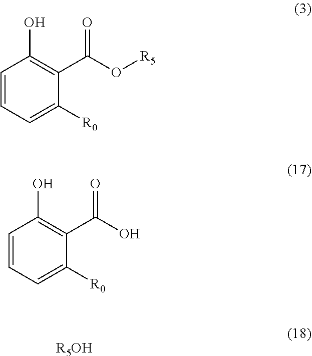
formulae (3) and (17) in which R₀ represent:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
R₀ optionally comprising one to six substituent(s) selected from —OH, —OR₇ or OCOR₇, wherein R₇ is an alkyl group of 1 to 3 carbon atoms
formulae (3) and (18) in which R₅ represents:
a linear alkyl group of 1 to 30 carbon atoms, or
a linear alkenyl group of 2 to 24 carbon atoms, comprising 1 to 6 alkenyl function(s), said alkenyl group being optionally substituted by a linear alkyl group of 1 to 5 carbon atom(s),
said R₅ group, in addition to the —OH group represented in formula (18), optionally comprising one to six substituent(s), in particular one or two, selected from —OH, —OR₇ and OCOR₇, wherein R₇ is an alkyl group of 1 to 3 carbon atom(s),
and further comprising a step of preparing J a compound of formula (11) from a compound of formula (3).
with the proviso that at least one of the R₀ and R₅ groups contains an alkenyl function
or wherein, in the composition comprising the primary plasticizer, the primary plasticizer is a compound of formula (12) 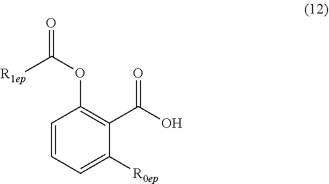
formula (12) in which R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3
R₀ₑₚ optionally comprising one to six substituent(s) selected from —OH, —OR₇ or OCOR₇, wherein R₇ is an alkyl group of 1 to 3 carbon atoms,
R₁ₑₚ represents:
a linear alkyl group of 1 to 30 carbon atom(s), optionally containing 1 to 6 epoxide function(s), or
a linear alkenyl group of 2 to 24 carbon atoms, optionally comprising 1 to 6 epoxide function(s), said alkenyl group being optionally substituted by an alkyl group of 1 to 5 carbon atom(s),
R₁ₑₚ optionally comprising one to six substituents, in particular one or two, selected from —OH, —OR₇ or OCOR₇, wherein R₇ is an alkyl group of 1 to 3 carbon atoms,
with the proviso that at least one of the R₀ₑₚ or R₁ groups contains at least one epoxide function,
and further comprising a step C of preparing a compound of formula (4) from a compound of formula (17) and an acid of formula (19): 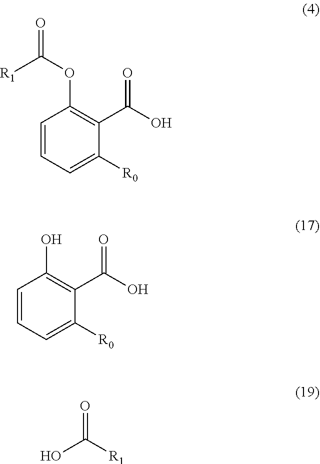
formulae (4) and (17) in which R₀ represent:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
R₀ optionally comprising one to six substituent(s) selected from —OH, —OR₇ or OCOR₇, wherein R₇ is an alkyl group of 1 to 3 carbon atoms
formulae (4) and (19) in which R₁ represents:
a linear alkyl group of 1 to 30 carbon atoms, or
a linear alkenyl group of 2 to 24 carbon atoms, comprising 1 to 6 alkenyl function(s), said alkenyl group being optionally substituted by a linear alkyl group of 1 to 5 carbon atom(s),
said R₁ group optionally comprising one to six substituent(s), in particular one or two, selected from —OH, —OR₇ and OCOR₇, wherein R₇ is an alkyl group of 1 to 3 carbon atom(s),
with the proviso that at least one of the R₀ and R₁ groups contains an alkenyl function,
and further comprising a step of preparing K a compound of formula (12) from a compound of formula (4);
or wherein, in the composition comprising the primary plasticizer, the primary plasticizer is a compound of formula (13) 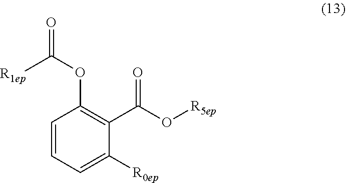
formula (13) in which R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
R₀ₑₚ optionally comprising one to six substituent(s) selected from —OH, —OR₇ or OCOR₇, wherein R₇ is an alkyl group of 1 to 3 carbon atoms
R₁ₑₚ and R₅ₑₚ independently represent:
a linear alkyl group of 1 to 30 carbon atom(s), optionally containing 1 to 6 epoxide function(s), or
a linear alkenyl group of 2 to 24 carbon atoms, optionally comprising 1 to 6 epoxide function(s), said alkenyl group being optionally substituted by an alkyl group of 1 to 5 carbon atom(s),
R₁ₑₚ and R₅ₑₚ optionally comprising one to six substituent(s), in particular one or two, selected from —OH, —OR₇ or OCOR₇, wherein R₇ is an alkyl group of 1 to 3 carbon atoms,
with the proviso that at least one of the R₀ₑₚ, R₁ₑₚ or R₅ₑₚ groups contains at least one epoxide function,
and further comprising a step of preparing D a compound of formula (5) from a compound of formula (17) with an alcohol of formula (18) and an acid of formula (19), 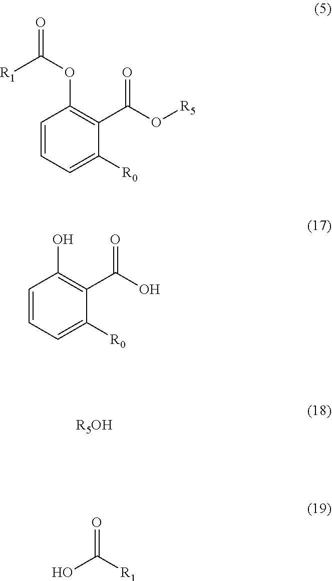
formulae (5) and (17) in which R₀ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
R₀ optionally comprising one to six substituent(s) selected from —OH, —OR₇ or OCOR₇, wherein R₇ is an alkyl group of 1 to 3 carbon atoms
formulae (5), (18) and (19) in which R₁ and R₅ independently represent:
a linear alkyl group of 1 to 30 carbon atoms, or
a linear alkenyl group of 2 to 24 carbon atoms, comprising 1 to 6 alkenyl function(s), said alkenyl group being optionally substituted by a linear alkyl group of 1 to 5 carbon atom(s),
said R₁ group, optionally comprising one to six substituent(s), in particular one or two, selected from —OH, —OR₇ and OCOR₇, wherein R₇ is an alkyl group of 1 to 3 carbon atom(s),
said R₅ group, in addition to the —OH group represented in formula (18), optionally comprising one to six substituent(s), in particular one or two, selected from —OH, —OR₇ and OCOR₇,
with the proviso that at least one of the R₀, R₁ or R₅ groups contains an alkenyl function
and further comprising a step of preparing L a compound of formula (13) from a compound of formula (5).

41. The process, according to claim 26, wherein, in the composition comprising the primary plasticizer, the latter is a compound of formula (14) 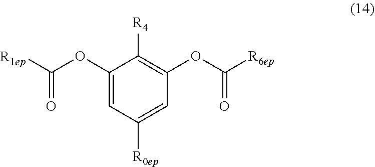
formula (14) in which R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
R₀ₑₚ optionally comprising one to six substituent(s) selected from —OH, —OR₇ or OCOR₇, wherein R₇ is an alkyl group of 1 to 3 carbon atoms,
R₄ is selected from:
a hydrogen atom,
a methyl CH₃,
R₁ₑₚ and R₆ₑₚ independently represent, in particular may be identical:
a linear alkyl group of 1 to 30 carbon atoms, optionally containing 1 to 6 epoxide functions, or
a linear alkenyl group of 2 to 24 carbon atoms, optionally comprising 1 to 6 epoxide function(s), said alkenyl group being optionally substituted by an alkyl group of 1 to 5 carbon atom(s),
R₁ₑₚ and R₆ₑₚ optionally comprising one to six substituent(s), in particular one or two, selected from —OH, —OR₇ or OCOR₇, wherein R₇ is an alkyl group of 1 to 3 carbon atoms,
with the proviso that at least one of the R₀ₑₚ, R₁ₑₚ or R₆ₑₚ groups contains at least one epoxide function,
and further comprising a step E of preparing the compound of formula (6) from a compound of formula (20) with an acid of formula (21) and an acid of formula (22): 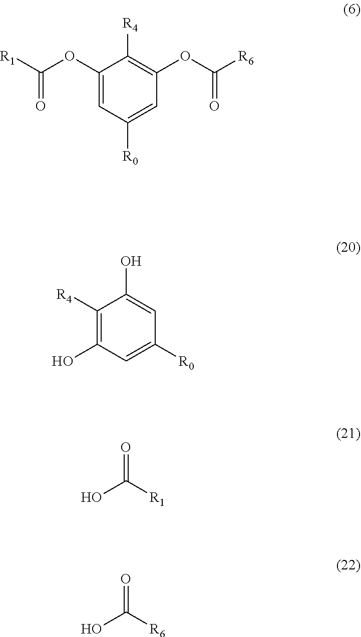
formulae (6) and (20) in which R₀ represent:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3
R₀ optionally comprising one to six substituent(s) selected from —OH, —OR₇ or OCOR₇, wherein R₇ is an alkyl group of 1 to 3 carbon atoms,
R₄ represents:
a hydrogen atom,
a methyl group CH₃,
formulae (6), (21) and (22) in which R₁ and R₆ independently represent:
a linear alkyl group of 1 to 30 carbon atom(s), or
a linear alkenyl group of 2 to 24 carbon atoms, comprising 1 to 6 alkenyl function(s), said alkenyl group being optionally substituted by a linear alkyl group of 1 to 5 carbon atom(s),
said R₄ group optionally comprising one to six substituent(s), in particular one or two, selected from —OH, —OR₇ and OCOR₇, wherein R₇ is an alkyl group of 1 to 3 carbon atom(s),
with the proviso that at least one of the R₀, R₁ or R₆ groups contains an alkenyl function,
and further comprising a step M of preparing the compound of formula (14) from a compound of formula (6).

42. The process, according to claim 26, wherein the compound of formula (1) is selected from compounds of the following formulae: 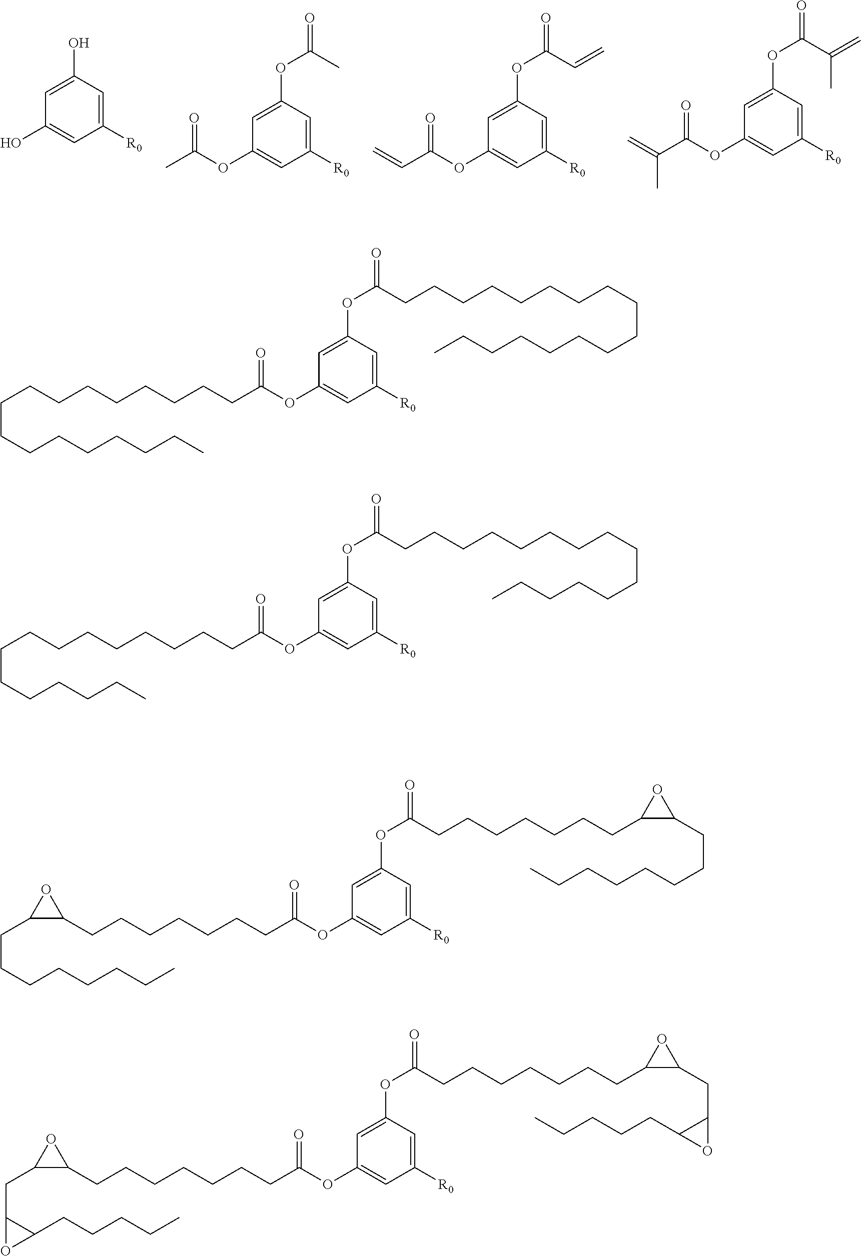 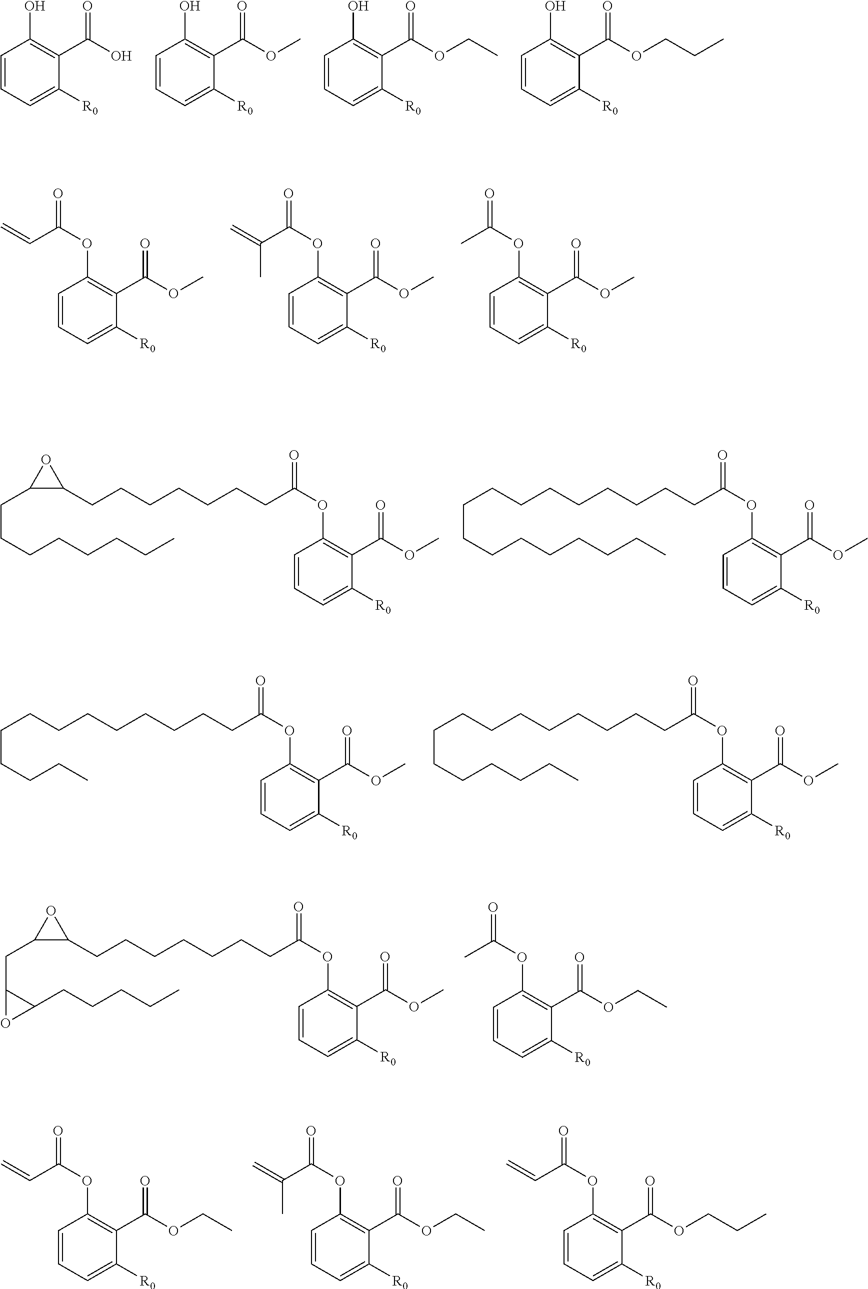 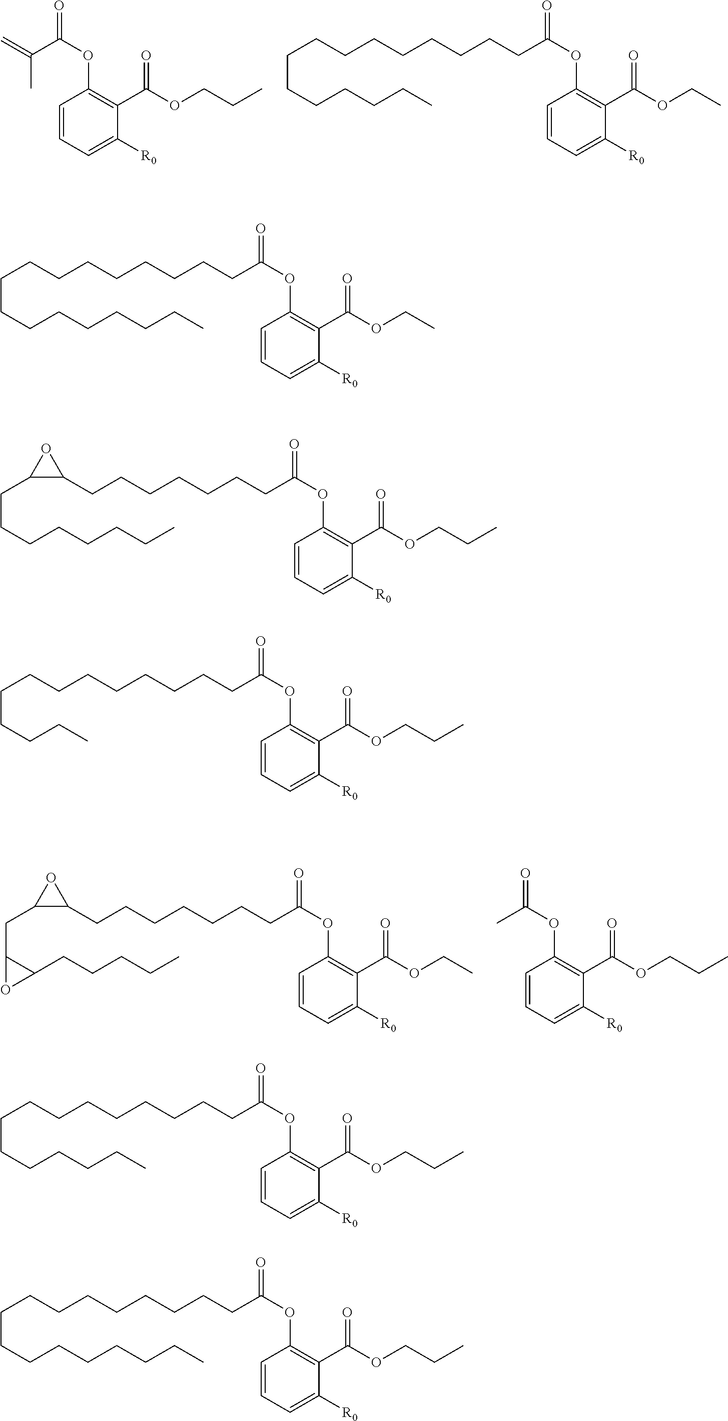 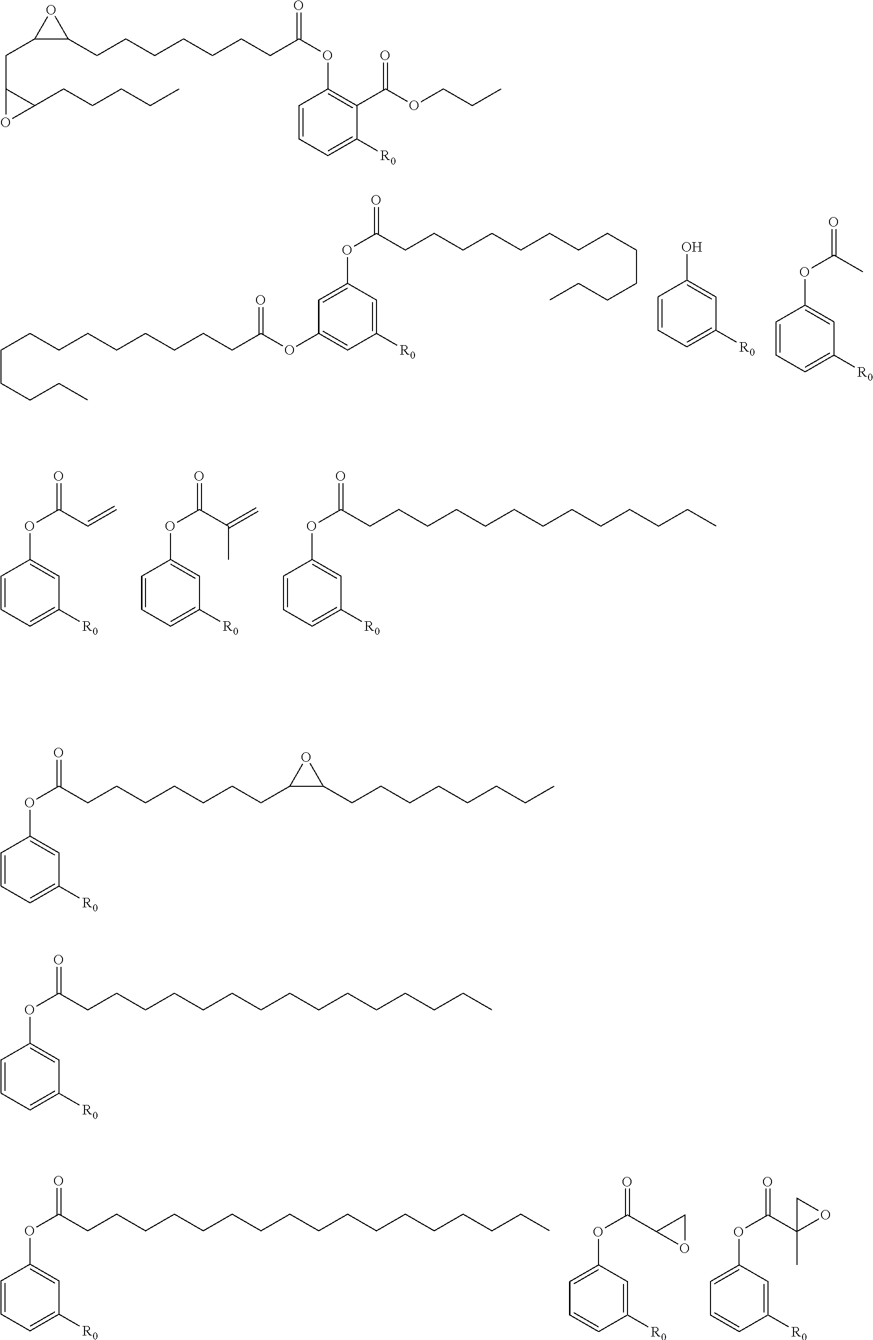 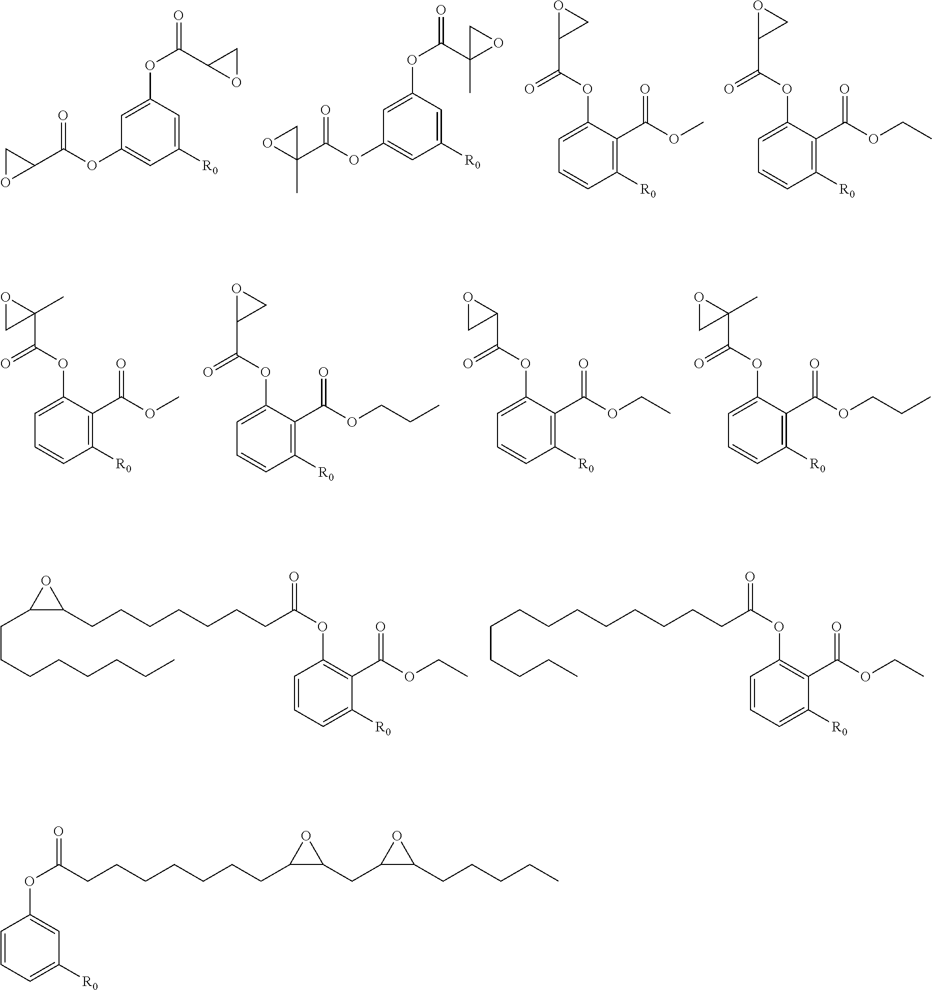
R₀ is selected from:

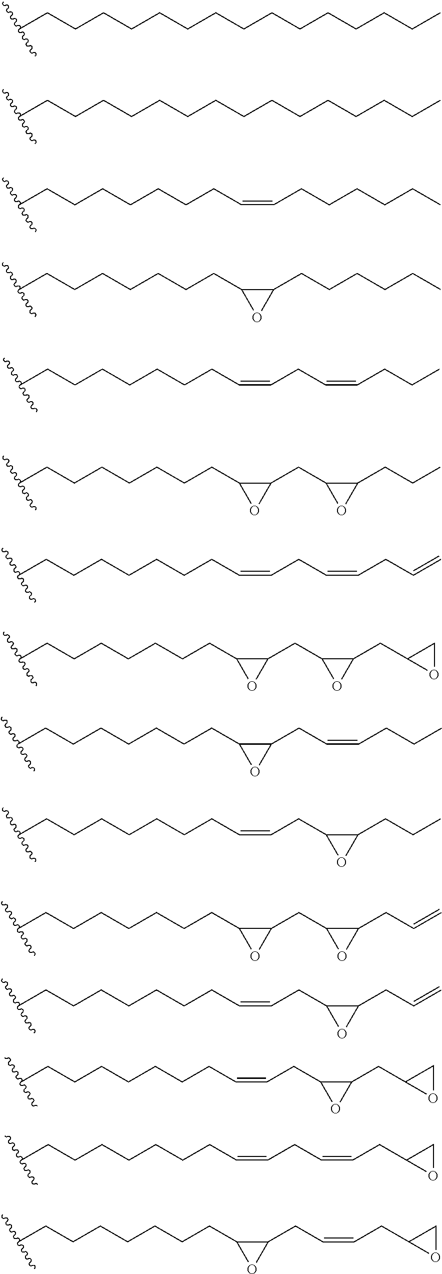

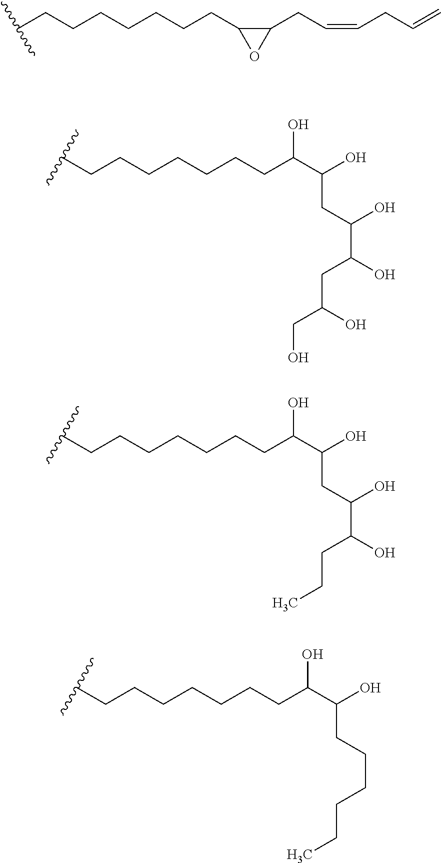

43. A plastic material comprising cellulose acetate obtainable by a process according to claim 26.

44. A compound, selected from the following formulae (11), (12), (13) and (14), 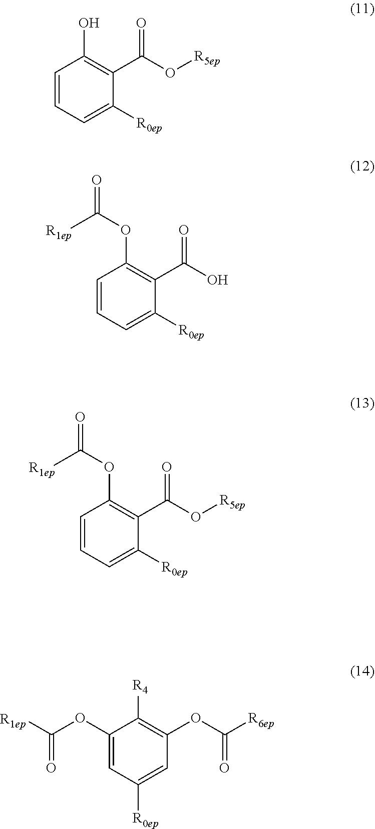
R₀ₑₚ represents:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
R₄ is selected from:
a hydrogen atom,
a methyl group CH₃
R₁ₑₚ, R₅ₑₚ and R₆ₑₚ are independently selected from:
an alkyl group of 2 to 30 carbon atoms, optionally containing 1 to 6 epoxide functions,
a linear alkenyl group of 2 to 24 carbon atoms, in particular comprising 1 to 6 epoxide functions, said alkenyl group being optionally substituted by an alkyl chain with 1 to 5 carbon atoms,
with the proviso that at least one of the R₀ₑₚ, R₁ₑₚ, R₅ₑₚ or R₆ₑₚ groups contains at least one epoxide function,
in particular where
R₀ₑₚ is selected from: 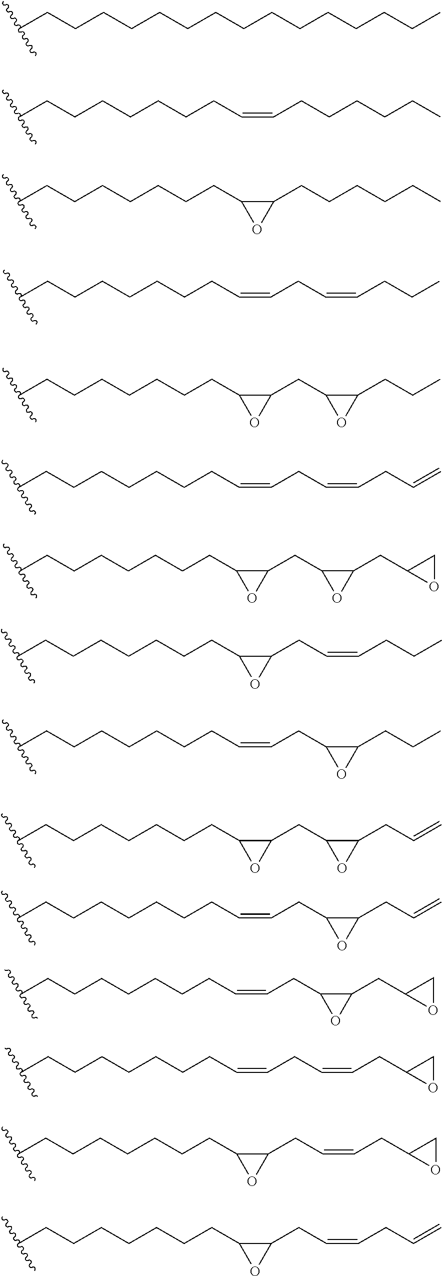
said R₀ₑₚ group optionally comprising 1 to 6 substituent(s) selected from —OH, —OR₇ and OCOR₇, wherein R₇ is an alkyl group of 1 to 3 carbon atom(s)
said R₁ₑₚ, R₅ₑₚ and R₆ₑₚ groups optionally comprising 1 to 6 substituent(s), in particular one or two, selected from —OH, —OR₇ and OCOR₇.

45. The compound according to claim 44, of formula (10) 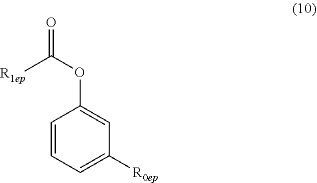
formula (2) in which
R₀ₑₚ is selected from:
a linear alkyl group of 15 carbon atoms optionally containing 1 to 3 epoxide functions, or
a linear alkenyl group of 15 carbon atoms, comprising x alkenyl functions, x varying from 1 to 3, comprising y epoxide functions, y varying from 0 to 2, x+y being less than or equal to 3,
R₁ₑₚ is selected from:
a linear alkyl group of 3 to 13 or 15 to 30 carbon atoms, optionally containing 1 to 6 epoxide functions, or
a linear alkenyl group of 2 to 17 or 19 to 24 carbon atoms, optionally comprising 1 to 6 epoxide function(s), said alkenyl group being optionally substituted by an alkyl group of 1 to 5 carbon atom(s),
R₀ₑₚ and R₁ₑₚ optionally comprising one to six substituent(s) selected from —OH, —OR₇ or OCOR₇, wherein R₇ is an alkyl group of 1 to 3 carbon atoms,
with the proviso that R₀ₑₚ or R₁ₑₚ contains at least one epoxide function.
